(19) United States
(12) Patent Application Publication
(10) Pub. No.: US 2026/0109998 A1
(43) Pub. Date: Apr. 23, 2026

(54) COMPOSITIONS AND METHODS FOR TREATING DUCHENNE MUSCULAR DYSTROPHY

(71) Applicant: The Trustees of the University of Pennsylvania, Philadelphia, PA (US)

(72) Inventor: Hansell Stedman, Norristown, PA (US)

(73) Assignee: The Trustees of the University of Pennsylvania, Philadelphia, PA (US)

(21) Appl. No.: 19/328,483

(22) Filed: Sep. 15, 2025

Related U.S. Application Data
(62) Division of application No. 17/047,990, now Pat. No. 12,460,226.
(60) Provisional application No. 62/658,464, filed on Apr. 16, 2018.

(51) Int. Cl. C12N 15/86; C07K 14/47

(52) U.S. Cl. CPC C12N 15/86; C07K 14/4708; C12N 2750/14121; C12N 2750/14122

(57) ABSTRACT

Described herein are triple-splice mutants of dystrophin or utrophin and methods of use thereof for treating Duchenne Muscular Dystrophy. Also provided are viral vectors which comprise a nucleic acid encoding a triple-splice mutant dystrophin or utrophin under the control of regulatory elements direct expression thereof. Compositions are also provided which contain such viral vectors formulated for delivery to a human patient.

### CROSS REFERENCE TO RELATED APPLICATION

This application is a divisional of U.S. patent application Ser. No. 17/047,990, filed Oct. 15, 2020, which is a National Stage Entry under 35 U.S.C. 371 of International Patent Application No. PCT/US2019/027675, filed Aug. 16, 2019, which claims priority to U.S. Provisional Patent Application No. 62/658,464, filed Apr. 16, 2018. Each of these applications is hereby incorporated by reference in its entirety.

### STATEMENT OF FEDERALLY SPONSORED RESEARCH

This invention was made with government support under NS094705 awarded by the National Institutes of Health. The government has certain rights in the invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “18-8438.US.D1.xml” (created Sep. 10, 2025 and 501,438 bytes in size).

### BACKGROUND OF THE INVENTION

Duchenne Muscular Dystrophy (DMD) is a severely disabling, systemic, childhood onset disease that progresses from early locomotive muscle weakness to end-stage respiratory and cardiomyopathy characterized by extraordinarily expensive caregiver and technology-dependence (Landfeldt, E., et al., The burden of Duchenne muscular dystrophy: an international, cross-sectional study. Neurology, 2014. 83 (6): p. 529-36; and Schreiber-Katz, O., et al., Comparative cost of illness analysis and assessment of health care burden of Duchenne and Becker muscular dystrophies in Germany. Orphanet J Rare Dis, 2014. 9: p. 210.). DMD is among the most common single-gene lethal diseases of mankind, with a historical global incidence of approximately 1:4000 male births. The molecular basis is the deficiency of the 427 kd isoform of cytoskeletal protein dystrophin (Dp427), which in the majority of cases is caused by multi-exon, frameshifting deletions in the DMD gene. A milder disease, Becker Muscular Dystrophy (BMD), is allelic, and most cases arise from internal deletions or duplications of the dystrophin gene that alter the length of the rod domain of the encoded protein.

Dystrophin was the first protein discovered by “positional cloning”, and this discovery provided the initial proof of concept for the use of a disease's human genetic map position as the primary basis for elucidating its molecular basis. Many proteins discovered by this approach are of very low abundance in the affected cells, complicating the ascertainment of the proteins' physiological function. Dystrophin was discovered in 1987, and 30 years on the field is faced with major gaps in the understanding of the protein's precise function in the muscle cell. However, it has been provided that indirect evidence of dystrophin's role in protecting muscle cell membranes from forces developed during muscle contraction. The nature of the mechanical loading of dystrophin remains poorly characterized.

Carrier detection and prenatal counseling have somewhat lowered the incidence of DMD in the USA (Pegoraro, E., et al., SPP1 genotype is a determinant of disease severity in Duchenne muscular dystrophy. Neurology, 2011. 76 (3): p. 219-26). Current therapy with the combination of glucocorticosteroids, ACE inhibitors, and mechanical ventilatory support may temporarily slow the rate of progression, but the ultimate clinical course is inexorable (McDonald, C. M., et al., The cooperative international neuromuscular research group duchenne natural history study-a longitudinal investigation in the era of glucocorticoid therapy: Design of protocol and the methods used. Muscle Nerve, 2013.).

In the era of gene therapy, various AAV vectors have emerged as the least toxic and most broadly disseminated platforms for systemic gene delivery. Those gene therapies hold great promise for systemic biodistribution, but limitations include (a) the cloning capacity limited to one third that required for full length Dp427, (b) unresolved issues of vector immunogenicity and toxicity at the doses potentially needed for durable therapy, and (c) the extraordinary cost of conventional manufacturing on a scale required for human therapy. AAV vectors are structurally related to the wild type members of the subfamily of parvovirus that encapsidate a single-stranded DNA genome of approximately 5 kilobases. The mRNA for full length dystrophin is 14 kilobases, with an open reading frame of approximately 12 kb.

The majority of mutations causing DMD are sporadic multi-exon, frameshifting deletions in the >2.5 megabase, X-linked gene (Kunkel, L. M., et al., Analysis of deletions in DNA from patients with Becker and Duchenne muscular dystrophy. Nature, 1986. 322 (6074): p. 73-7; Monaco, A. P., et al., Isolation of candidate cDNAs for portions of the Duchenne muscular dystrophy gene. Nature, 1986. 323 (6089): p. 646-50; and Koenig, M., et al., The molecular basis for Duchenne versus Becker muscular dystrophy: correlation of severity with type of deletion. Am J Hum Genet, 1989. 45 (4): p. 498-506.). In the absence of central (thymic) tolerance to the deficient full-length protein, recombinant dystrophin has the capacity to induce host immune responses to the foreign protein (Mendell, J. R., et al., Dystrophin immunity in Duchenne's muscular dystrophy. N Engl J Med, 2010. 363 (15): p. 1429-37). Novel vectors and vascular delivery methods have achieved promising regional and systemic gene transfer in proof of-concept preclinical studies, suggesting rational approaches to gene therapy for DMD (Greelish, J. P., et al., Stable restoration of the sarcoglycan complex in dystrophic muscle perfused with histamine and a recombinant adeno-associated viral vector. Nat Med, 1999. 5 (4): p. 439-43; Su, L. T., et al., Uniform scale-independent gene transfer to striated muscle after transvenular extravasation of vector. Circulation, 2005. 112 (12): p. 1780-8; Gao, G., L. H. Vandenberghe, and J. M. Wilson, New recombinant serotypes of AAV vectors. Curr Gene Ther, 2005. 5 (3): p. 285-97; and Katz, M. G., et al., Cardiac gene therapy: optimization of gene delivery techniques in vivo. Hum Gene Ther, 2010. 21 (4): p. 371-80.). These developments also, however, highlight the major vector discovery challenges and patient safety concerns in this field (Mendell, J. R., et al., Dystrophin immunity in Duchenne's muscular dystrophy. N Engl J Med, 2010. 363 (15): p. 1429-37; Mendell, J. R., et al., Myoblast transfer in the treatment of Duchenne's muscular dystrophy. N Engl J Med, 1995. 333 (13): p. 832-8; Mouly, V., et al., Myoblast transfer therapy: is there any light at the end of the tunnel? Acta Myol, 2005. 24 (2): p. 128-33; and Wang, Z., et al., Immunity to adeno-associated virus-mediated gene transfer in a random-bred canine model of Duchenne muscular dystrophy. Hum Gene Ther, 2007. 18 (1): p. 18-26.). As another example of AAV vectors treating DMD, U.S. Pat. No. 7,771,993 provides for a “micro-utrophin” (also noted as “m-utrophin”, “μ-utrophin” or “μ-U”) having a functional portion of the “actinin-binding domain” of about 270 amino acids relative to the human utrophin which is located within the N-terminal utrophin region, at least functional portions of the proline-rich hinge regions 1 and 4 (H1) and (H4), and a portion of the C-terminal utrophin protein. The micro-utrophin contains internal deletions of the central rod repeat domains and a truncation in the C-terminal region downstream.

There remains a need for treatment of Duchenne Muscular Dystrophy and related diseases.

### SUMMARY OF THE INVENTION

The present invention provides compositions and methods useful for treatment of Muscular Dystrophy (MD), including Duchenne Muscular Dystrophy (DMD) and Becker Muscular Dystrophy (BMD), and other diseases. Provided herein is a recombinant adeno-associated virus (rAAV) vector having an AAV capsid and a vector genome. The vector genome comprises a nucleic acid sequence encoding a dystrophin superfamily triple splice mutant protein under control of regulatory sequences which direct expression thereof.

In certain embodiments, the dystrophin superfamily triple splice mutant protein comprises a hybrid helical domain comprising a first helix comprising an N-terminal portion of a helix A fused to a C-terminal portion of a helix A′, a second helix comprising an N-terminal portion of a helix B′ fused to a C-terminal portion of a helix B, and, a third helix comprising an N-terminal portion of a helix C fused to a C-terminal portion of helix C′, wherein helices A, B, and C are present in a first triple helical repeat that is non-adjacent to a second triple helical repeat having helices A′, B′, and C′ in a native dystrophin superfamily protein. In certain embodiments, the dystrophin superfamily mutant protein is a triple splice mutant dystrophin or triple splice mutant utrophin.

In yet further embodiments, the dystrophin superfamily triple splice mutant protein comprises N-terminal helical repeat(s), a hybrid triple helical repeat, and C-terminal helical repeat(s), wherein total number of the helical repeats including the hybrid repeat in the triple splice mutant protein is selected from any integer of 1 to 1 less than the helical repeat number of the full-length dystrophin superfamily protein, and wherein the hybrid triple helical repeat are formed by two helical repeats spliced on the plane that bisects the helical repeat perpendicular to its long axis as depicted in FIG. 2F. In certain embodiments, the dystrophin superfamily mutant protein is a triple splice mutant dystrophin or triple splice mutant utrophin.

Novel recombinant mutant dystrophins having the amino acid sequence of SEQ ID NO: 1 or 22 are provided, and novel recombinant mutant utrophins having the amino acid sequence selected from the group consisting of SEQ ID NOs: 3, 7, and 20 are also provided. In certain embodiments, the triple splice mutant protein is utrophin and encoded by a nucleic acid comprising SEQ ID NO: 19, or a sequence about 95% to about 99% identical thereto.

In further embodiments, the triple splice mutant protein comprises a hybrid triple helical repeat and C-terminal helical repeats, wherein total number of the helical repeats including the hybrid repeat in the triple splice mutant protein is five, and wherein the hybrid triple helical repeat is formed by two helical repeats spliced on the plane that bisects the helical repeat perpendicular to its long axis as depicted in FIG. 2F. In certain embodiments, the C-terminal helical repeats of the mutant protein consist of helical repeats 21, 22, 23, and 24 in full-length dystrophin, wherein helical repeat 1 of the two repeats forming the hybrid triple helical repeat is helical repeat 1 in full-length dystrophin, and wherein helical repeat 2 of the two repeats forming the hybrid triple helical repeat is helical repeat 20 in full-length dystrophin. In yet another embodiment, the C-terminal helical repeats of the mutant protein consist of helical repeats 19, 20, 21, and 22 in full-length utrophin, wherein helical repeat 1 of the two repeats forming the hybrid triple helical repeat is helical repeat 1 in full-length utrophin, and wherein helical repeat 2 of the two repeats forming the hybrid triple helical repeat is helical repeat 18 in full-length utrophin.

In yet further embodiments, novel recombinant mutant dystrophin protein comprising an amino acid sequence of SEQ ID NO: 1, 13, 14, 15, 16, 17, 18, or 22 are provided. In certain embodiments, nucleic acids encoding mutant dystrophin superfamily proteins are provided. In a yet a further embodiment, plasmids comprising nucleic acids encoding a mutant dystrophin superfamily protein are provided.

In certain embodiments, pharmaceutical compositions comprising a rAAV comprising a vector genome comprising a nucleic acid sequence encoding a triple splice mutant dystrophin superfamily protein are provided.

In yet further embodiments, methods of treating a subject diagnosed with Duchenne muscular dystrophy comprising administering a pharmaceutical composition comprising a rAAV comprising a vector genome having a nucleic acid sequence encoding a triple splice mutant dystrophin superfamily protein are provided.

Other aspects and advantages of the present invention will be apparent from the following Detailed Description of the Invention.

### BRIEF DESCRIPTION OF THE DRAWINGS

FIG. 1A to FIG. 1D provide an illustration of forming a hybrid triple helix domain. FIG. 1A shows components of an exemplified dystrophy superfamily protein. From the N-terminal (indicated as NH₂—) of the protein to the C-terminal (indicated as —COOH), the components are 1) two calponin homology domains (CH1 and CH2), 2) several triple helix domains (noted as TH followed by a number), 3) a modular, multi-domain globular region (WW-EF-ZZ) and 4) an extreme C-terminal region. In this illustration, there are 24 triple helix domains. Grey arrowheads indicate splice junction site utilized in previous formation of a shorten dystrophin superfamily protein (such as described in U.S. Pat. No. 7,771,993, which is incorporated by reference herein in its entirety), while black arrowheads indicate splice junction site used to form a hybrid triple helix domain. FIG. 1B provides a closer look at the triple helix domains, THn in white arrows and THn′ in shaded arrows, which are spliced to form a hybrid triple helix as shown in FIG. 1C. Each white arrow or shaded arrow in FIG. 1B to FIG. 1D indicates a helix in a domain (Helix A, B, and C for THn; Helix A′, B′ and C′ for THn′; and Helix A-A′, B′-B and C-C′ in the hybrid triple helix domain). The black arrow lines indicate the splice junction sites. FIG. 1D provides a linear display of the hybrid triple helix domain. The white section of the bars/arrows indicate that they are originally from THn while the shaded section indicated that they are originally from THn′. As shown in FIG. 1D, the first helix of the hybrid triple helix domain comprises Helix A in the N-terminal and Helix A′ in the C-terminal; the second helix of the hybrid triple helix domain comprises Helix B′ in the N-terminal and Helix B in the C-terminal; and the third helix of the hybrid triple helix domain comprises Helix C in the N-terminal and Helix C′ in the C-terminal.

FIG. 2A to FIG. 2F illustrate the utrophin modifications provided herein. The lighter colored residues depict the tryptophans, which are the most conserved residues in the Hidden Markov Modelling (HMM) for the triple helical repeat domains shared by α-actinin, β- and α-spectrin, dystrophin, utrophin and the spectroplakins. The sequence divergence at all other positions with extensive evolutionary coupling establishes that these regions of individual repeats are not interchangeable from one repeat to another without destabilizing the triple helix. No single splice can resolve this problem, but a triple spice across the plane of the tryptophan residues can, by retaining the coupled amino acid left and right of the plane. FIG. 2A illustrates a portion of the triple helical repeat domain (TH1) of α-actinin. FIG. 2B illustrates a TH2 portion of α-actinin. FIG. 2C illustrates a TH3 portion of α-actinin. FIG. 2D illustrates a TH4 portion of α-actinin. FIG. 2E illustrates a β- and α-spectrin tet site. FIG. 2F illustrates a triple splice across the plan of the tryptophan residues, by retaining the coupled amino acid left and right of the plane (to help with orientation, the tryptophans are shown twice).

FIG. 3 shows part of the HMM logo of Spectrin family (PF00435). More details can be found at pfam.xfam.org/family/PF00435 #tabview=tab4. This HMM logo provides a quick overview of the properties of an HMM in a graphical form. Relative conservation of each of two anchoring tryptophans, as depicted in the HMM logo for spectrin-like triple helical repeats at position #16. One of skill in the art can find out how to interpret the logo as described for example, in Schuster-Böckler B et al, HMM Logos for visualization of protein families. BMC Bioinformatics. 2004 Jan. 21; 5:7.

FIG. 4 provides an overview of forming a nano-dystrophin as described herein as well as a comparison with forming a micro-dystrophin.

FIG. 5 provides a grid showing possible splice junctions in Helix B of dystrophin. More detailed description regarding this drawing can be found in the first paragraph of the Detailed Description of the Invention, I. Dystrophin, utrophin and others, A. Splice junctions in the helices. The variants depicted correspond to the nano-dystophin amino acid sequences of SEQ ID NOs: 13-18. Row 1: SEQ ID NO: 23, Row 17: SEQ ID NO: 24.

FIG. 6A to FIG. 6C show that the length of the Dystrophin rod was established prior to the emergence of sarcomeres. FIG. 6A shows human dystrophin gene, mRNA, and protein domain structure aligned to demonstrate intron position and phasing. 22 of 24 spectrin repeats of human dystrophin contain a phase 0 intron at HMM position 46 (dots with black outline). Cnidarian dystrophin shares rod-domain phase 0 introns (for clarity only HMM position 46 phase 0 introns are depicted in the cnidarian mRNA). FIG. 6B provides human beta heavy spectrin, dystrophin, and MACF1 depicted with the rod domains vertically aligned to a consensus sequence for the triple helical domain HMM. Superimposed are the positions and phases of introns relative to the corresponding coding sequences, clearly showing the structural similarity of the dystrophin and MACF1 genes. For clarity, only introns shared with orthologous genes from the most distantly related species are shown, with the spectrin gene showing vestigial introns (arrow) from a remote partial gene duplication of 13 repeats (spectrin—A. queenslandica; dystrophin—N. vectensis; MACF1—T. adhaerens). FIG. 6C provides a phylogenomic distribution of alpha-actinin protein superfamily members in select eukaryotic lineages. The number of rod-domain spectrin repeats is specified in parenthesis. Members of the dystroplakin superfamily harbor phase 0 introns at HMM position 46, whereas alpha-actinin and spectrin family proteins lack conservation of a phase 0 intron at this position. HMM 46 intron-driven expansion of the ancestral MACF1 ortholog is observed between fungi and Placozoa. The Ancestral MACF1 ortholog underwent a partial gene duplication that donated an N-terminal actin binding domain and a full-length rod domain to an ancestral WW-EF-ZZ dystrophin ortholog.

FIG. 7A to FIG. 7F show that widespread transduction restores the Dystrophin Associated Protein Complex, prevents myofiber degeneration, normalizes serum CK level and improves muscle function in AAV9-μUtrophin treated mdx mice. As shown in FIG. 7A, Immunostaining of representative limb muscle for epitopes shared by native and recombinant Utrophin (Utro_N), for epitopes unique to native Utrophin (Utro_C), γ-sarcoglycan, embryonic myosin heavy chain (eMHC), and laminin; scale bar 25 μm. FIG. 7B provides H&E of representative limb muscles showing suppression of myonecrosis and mononuclear cell infiltration; scale bar 100 μm. FIG. 7C provides western blot analysis detecting expression of recombinant utrophin (AAV9-uUtro) and γ-sarcoglycan (γ-sarc). FIG. 7D provides percentage of centrally nucleated myofibers (CNF), statistical measures as defined in methods (Color=distinct animal, shape=distinct muscle, same color/shape=technical replicate). FIG. 9E provides measurement of serum CK levels in treated mice (n=5), untreated mice (n=12) (***p<0.0001), and N.S. from wild type (n=7). FIG. 7F provides quantification of vertical activity one hour post-grip strength in treated mice (n=8), untreated mdx mice (n=11), and wild type mice (n=6). Error bars represent SD, **p<0.001; N.S indicates not significant; statistical significance was assessed by Kruskal-Wallis test with multiple group comparison.

FIG. 8A to FIG. 8G show that systemic delivery of AAV9-μUtrophin in GRMD dogs at 7 weeks of age prevents myonecrosis and results in rapid reduction of serum CK levels. FIG. 8A provides an experimental timeline. FIG. 8B provides representative H&E of vastus lateralis and temporalis muscle showing abundant myonecrotic fibers and mononuclear cell infiltration in untreated muscle, while treated muscles resemble WT. FIG. 8C provides Alizarin red S staining showing calcified fibers indicating muscle degeneration (left panel, red), with corresponding quantification (FIG. 8E). FIG. 10D provides immunofluorescent staining with f1.652 showing cluster of eMHC-positive fibers (right panel, red), with corresponding quantification (FIG. 8F). FIG. 8G provides serum creatine kinase (CK) levels at various time points pre/post systemic AAV9-μUtrophin infusion. Scale bar 100 μm.

FIG. 9A to FIG. 9D show that widespread expression of μUtrophin rescues the dystrophin-associated protein complex proteins in treated GRMD dogs after systemic delivery at age of 7 weeks. FIG. 9A and FIG. 9B provide immunofluorescent staining of representative limb muscle. FIG. 9A shows native and recombinant Utrophin (Utro_N), native Utrophin (Utro_C), laminin. FIG. 9B shows β-dystroglycan (green), β-sarcoglycan (green) and γ-sarcoglycan (red). Scale bar 100 μm. FIG. 9C shows a western blot analysis showing widespread biodistribution of μUtrophin (˜135 kD) in striated muscle at necropsy. FIG. 9D shows a western blot analysis showing expression of β-dystroglycan in muscle biopsies of Vastus Lateralis (VL) and cranial sartorius (CS). Treated (H)/Treated (B) indicates tissue from treated dogs, Hann and Beetle.

FIG. 10A to FIG. 10D show that focal expression of μDystrophin, but not μUtrophin elicits a detectable peripheral and local immune response in a dystrophin deletional-null dog model. FIG. 10A provides an experimental timeline. Dystrophin deletional-null dogs (Grinch and Ned) each received IM injections of AAV9-μDystrophin (Right) and AAV9-μUtrophin (Left) at equivalent doses (1×10¹² vg/kg) into their tibialis anterior compartment. As shown in FIG. 10B, PBMCs were collected pre-, 2, 4, 6, 8 weeks post-injection and cultured with synthetic peptides spanning the entire μDystrophin (Pool A-D) and μUtrophin (Pool E-J) peptide sequences, while vaccine peptides and PMA/Ion served as positive controls. A positive result was interpreted as ≥5 spot forming units (SFU)/1E5 PBMCs (dotted line). FIG. 10C shows immunofluorescent (green) staining of muscle biopsies collected 4 weeks post-injection against Utro N (top row) and dystrophin (bottom row). Inset with red border—for reference, appearance of normal muscle stained green for dystrophin. FIG. 10D shows representative H&E of muscle biopsies collected 4 weeks post-injection

FIG. 11 provides an overview of micro-utrophin. The site of deletion junction (splice junction) is indicated. Hinge 1, 2, and 4 are labelled as H1, H2, and H4. SR1, SR2, SR3 and SR22 correspond to TH1, TH3, TH3 and TH22 of a full-length utrophin.

FIG. 12A to FIG. 12D provide a design of a hybrid test: vertical activity monitoring and the whole limb force test. FIG. 12A provides a schematic diagram showing that experimental timeline. FIG. 12B shows quantification of vertical activity in the open filed cage between c57 (n=11) and mdx mice (n=12) before the whole limb force test showed no significant difference between c57 and mdx mice (P>0.05 Mann-Whitney test). FIG. 12C shows that the whole limb force test was conducted over a series of seven pulls for both c57 (n=11) and mdx mice (n=17). C57 mice are indicated with a square and mdx mice are indicated with a circle. The distribution of whole limb force for each series of pulls is demonstrated. Equations are depicted (P<0.0001, 2-way ANOVA test). FIG. 12D provides an analysis of cumulative post-vertical activity for 1 h following the force test showed that there are significant differences between c57 and mdx mice (P<0.0001, 2-way ANOVA).

FIG. 13 provides a visual representation of the locomotor activity of both c57 and mdx mice for 5 min before the whole limb force test (Pre), as well as the first 5 min and the second 5 min after the force test. Lines represent horizontal activity, and dots represent vertical activity.

FIG. 14A to FIG. 14G show that acute sarcolemmal disruption in clustered myocytes of BIO 14.6 hamster skeletal muscle after forceful contraction. FIG. 14A provides simultaneous view of Evans blue dye and dystrophin counterstain in muscle fibers 72 h after i.v. injection of Evans blue dye. In FIG. 14B, as viewed through a FITC filter, complete absence of dystrophin staining is apparent in all Evans blue-positive fibers in section from FIG. 14A. FIG. 14C shows that myocyte injury leads to loss of dystrophin in most Evans blue-positive fibers within 8 h of a single bout of voluntary running. FIG. 14D shows Tibialis anterior muscle 3 h after tetanic contracture at a lengthening rate of 0.75 muscle lengths per second. Acute injury is shown by procion orange dye uptake; dystrophin counterstaining indicates the absence of complete membrane disintegration and non-specific proteolysis in these fibers. FIG. 14E shows dystrophin counterstain of muscle cryosection after 1 h of running wheel exercise. FIG. 14F shows Evans blue dye fluorescence in same section as in FIG. 14E. FIG. 14G provides Alizarin red S stain of same region of serial cryosection. Original magnifications: FIGS. 14A, 14B and 14E to 14G, 100×; FIG. 14C and FIG. 14D, 200×.

FIG. 15 provides an analysis of dystrophin-associated protein complex glycosylases, ligands, Actinin Spectrin superfamily, motor proteins and titin-obsurin superfamily. More discussion regarding this figure can be found in Example 3.

FIG. 16A and FIG. 16B provide a model of adjacent triple helical repeats of human utrophin using templates derived from human beta2-spectrin (3EDV, FIG. 16A) and human plectin (5J1G). More details can be found in Example 3.

FIG. 17 provides an overview of micro-Utrophin. This micro-Utrophin juxtaposes an unstructured, proline-rich inter-helical “hinge-2” domain (H2) against the last triple helical repeat, number 22 of full length Utrophin. R1, R2, R3 and R₂₂ correspond to TH1, TH3, TH3 and TH22 of a full-length utrophin. More details can be found in Example 3, Section F.

FIG. 18 provides expression level of the optimized micro-Utrophin as described in Example 3.

FIG. 19 shows robust dose-dependent μUtrophin expression and stabilization of wild type levels of sarcoglycan expression in the sarcolemma six weeks post-injection in the GRMD dogs as described in Example 3.

FIG. 20(I) and FIG. 20(II) show μUtrophin specific T cell response in injected GRMD dogs as discussed in the Examples 2 and 3. Peripheral blood mononuclear cells (PBMCs) collected at 5 and 8 weeks post injection were cultured with three pools of synthetic peptides corresponding to the AAV9 capsid (A, B, and C) as well as five pools of synthetic peptides spanning the entire μUtrophin peptide sequence (A-B, C-D, E-F, G-H, I-J). y-interferon production was assessed by counting the spot forming units per million PBMCs, with no response above background against AAV9 capsid or utrophin-derived peptide pools in injected dogs. Lower right, control assay following Adenovirus-CMV-lacZ injection, showing positive response (asterisk) to both Had5 (1-4) and lacZ (5-8) peptide pools based on the most conservative interpretation of a positive result (dotted line).

FIG. 21 shows a 79 kd band in load-bearing muscles of AAV-μ-U injected mdx mice as discussed in Example 3, Section G. Lanes 1, 2, and 4: non-load-bearing (e.g. flexor) muscles; 3, 5, and 7: load-bearing (e.g. extensor) muscles; 6 liver; 8 PBS-injected mdx muscle; 9, molecular weight marker.

FIG. 22(I) and FIG. 22 (II) show a combination of western blotting, immunoaffinity purification, and LC/MS-MS identifying a 79 kd fragment as the N-terminal portion of micro-Utrophin (SEQ ID NO: 25). The box encompasses the sequence PPPPP, a portion of “Hinge 2” immediately upstream of the deletional junction, as shown.

FIG. 23 shows that SH3 domain makes multiple high affinity contacts with compatible amino acid side chains from the adjacent triple helices on both sides, a configuration with the potential to transmit longitudinal force and resist unfolding.

FIG. 24A to FIG. 24C show that Anc80 achieves a global biodistribution of micro Utrophin comparable to that of AAV9 in this context, with strong transduction of cardiac and skeletal muscle (see micrographs in FIG. 24A and FIG. 24B, and western blots in FIG. 24C) as discussed in Example 3.

FIG. 25 shows a qualitative comparation of AAV9 and Anc80 for biodistribution of μUtrophin in cohorts of mdx mice following systemic administration of these vectors in equal doses of 2.5×10¹² vg/mouse. Representative western blots from multiple muscles from two mice for each vector are shown, demonstrating widespread and efficient transduction of striated muscles with both vectors. The uppermost band is μUtrophin, as labeled with a polyclonal antibody that specifically recognizes an epitope corresponding to the protein's N-terminus. The sample loading control is the lowermost band labeled with an antibody to the protein vinculin. Represented striated muscles: Diaphragm, Triceps, Quadriceps, Gastrocnemius, Abdominal wall, Pectoralis, Tibialis Anterior, Heart.

FIG. 26A and FIG. 26B show that AAV9-μUtrophin eliminates MuRF-1 (+), TUNEL (+) and centrally nucleated myofibers and in mdx mouse muscle. In FIG. 26A, Images as labeled are representative of 8-week old mdx mice injected as neonates with either AAV9 μU or PBS. Histological staining with MuRF-1 and TUNEL serves as biomarker for active proteolysis and apoptosis respectively. FIG. 26B provides a Table summarizing mean and standard deviation of centrally nucleated myofibers, MuRF-1, TUNEL and embryonic positive myofibers in mdx PBS-treated, mdx AAV9-μUtrophin treated and c57 wild type PBS-treated group (n=3). Central nucleation in mdx muscle fibers is indicative of at least one previous episode of necrosis followed by regeneration as mdx mice reach 8 weeks of age.

FIG. 27 provides that normal growth of GRMD dogs randomized to AAV9-μUtrophin as evidence against immune-mediated myositis. Individual weights of dogs randomized to the highest doses (1×10^{13.5} vg/kg) of AAV9-cU (canine μ-Utrophin) without immunosuppression, as well as relevant controls including littermate randomized to PBS and other littermate carrier females and non-littermate GRMD males and females. Also included for comparison are relevant weights of previously reported GRMD females receiving AAV9—hD (human μ-Dystrophin) showing rapid weight loss immediately prior to euthanasia and necropsy showing signs of systemic myositis (Kornegay, J. N., et al., Widespread muscle expression of an AAV9 human mini-dystrophin vector after intravenous injection in neonatal dystrophin-deficient dogs. Mol Ther, 2010. 18 (8): p. 1501-8.).

FIG. 28 shows a western blot from a study comparing the stability of micro-utrophin and nano-utrophin in vivo. Mdx mice were injected with AAV vectors encoding either micro-utrophin or nano-utrophin followed by detection of the protein (including a 79 kd N-terminal subfragment) in muscle tissue.

FIG. 29A and FIG. 29B show alternate viewpoints of a 3-D rendition of a mutant hybrid helical repeat formed by splicing TH 1 and TH 20 of full-length dystrophin. The splice in the B antiparallel helix is positioned in the plane with the W residues (yellow) on the parallel A and C helices.

FIG. 30A and FIG. 30B show alternate viewpoints of a 3-D rendition of a mutant hybrid helical repeat formed by splicing TH 1 and TH 18 of full-length utrophin. The splice in the B antiparallel helix is positioned in the plane with the W residues (yellow) on the parallel A and C helices.

### DETAILED DESCRIPTION OF THE INVENTION

Compositions and methods are provided for treating Muscular Dystrophy (MD), including Duchenne Muscular Dystrophy (DMD) and Becker Muscular Dystrophy (BMD), and other related disease, via a dystrophin superfamily triple splice mutant protein, a nucleic acid sequence encoding thereof, or a vector comprising such nucleic acid sequence.

An unusual molecular evolution analysis of dystrophin and its supergene family was performed, leading to a paradigm shift. The inventors' analysis indicates that dystrophin's primary role is that of a strong, tethering, non-extensible, “strut”-like rod, not a shock absorber as previously theorized; that its length is secondary in importance to its strength; that its strength depends, throughout the rod domain which accounts for 80% of the protein's primary structure, on the interaction of amino acids at the boundaries between adjacent “triple-helical”, “spectrin-like” repeats; that dystrophin is only as strong as its weakest link; that evolution has selected against internal deletions that weaken the protein. Thus, in order to shorten, without weakening the protein, is to cut the coding sequence for the polypeptide at multiple sites that can be aligned in the folded protein across the central triple helical domains of greatest sequence conservation (e.g. in dystrophin across the 2-dimensional plane that bisects the interacting tryptophan residues in the hydrophobic core). This is the strongest element in the Hidden Markov Models for all spectrin-like triple helical repeats, and applies to most of the repeats of dystrophin, utrophin, and the spectroplakins (MACF, dystonin, etc.)

### I. Dystrophin, Utrophin and Others

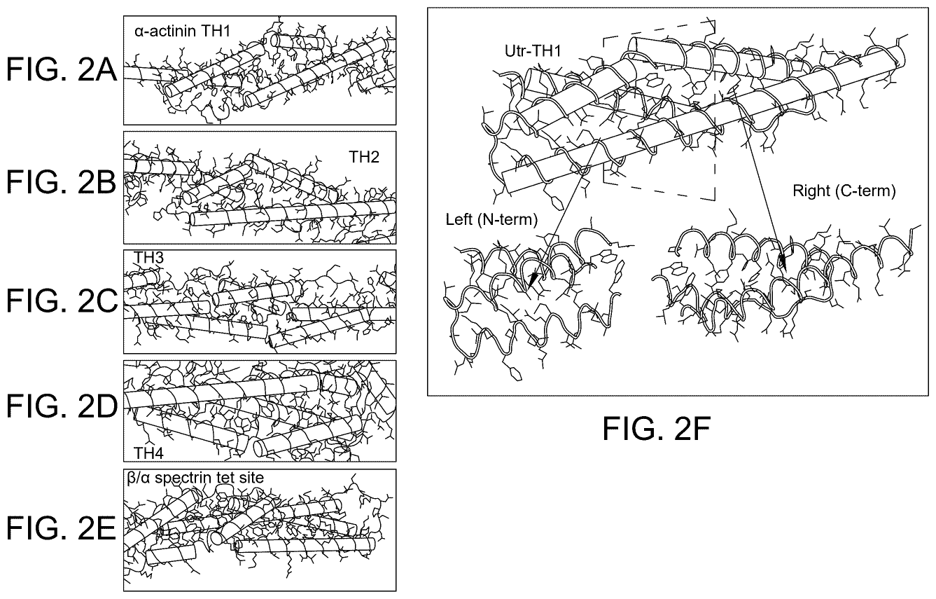

As used herein, a dystrophin superfamily protein refers to a protein comprising a “spectrin-like” and “rod-like” domain which consists of three α-helices and occurs as either single copies or in tandem arrangements of multiple repeats in the protein, such as dystrophin, utrophin, alpha-actinin, alpha-spectrin, beta-spectrin, or other members of spectrin family, plakins, spectraplakins (i.e., spectroplakins). The three-α-helix domain comprises two similarly (Helices A and C) and one oppositely (Helix B) directed α-helices joined by nonhelical linkers. A number of aromatic residues in the hydrophobic core of the domain are typically conserved. See, e.g., Parry D A et al. Analysis of the three-alpha-helix motif in the spectrin superfamily of proteins. Biophys J. 1992 April; 61 (4): 858-67; and Djinovic-Carugo K et al, The spectrin repeat: a structural platform for cytoskeletal protein assemblies. FEBS Lett. 2002 Feb. 20; 513 (1): 119-23. Examples of such repeats can be found in FIGS. 2A to 2E. In certain embodiments, the full-length dystrophin superfamily protein refers to a dystrophin superfamily protein or an isoform thereof, which may exist in a healthy control. In certain embodiments, the full-length dystrophin superfamily protein refers to the dystrophin superfamily protein or an isoform thereof considered as a canonical sequence by one of skill in the art. Such canonical sequences are available, e.g., on the world wide web at uniprot.org.

Spectrin is a cytoskeletal protein that lines the intracellular side of the plasma membrane in eukaryotic cells. Spectrin forms pentagonal or hexagonal arrangements, forming a scaffolding and playing an important role in maintenance of plasma membrane integrity and cytoskeletal structure. See, e.g., Huh G Y et al, Calpain proteolysis of alpha II-spectrin in the normal adult human brain. Neurosci Lett. 2001 Dec. 4; 316 (1): 41-4. Proteins in this superfamily can be defined by two features: (1) an N-terminal actin-binding domain; and (2) a section of α-helical spectrin repeats. See, e.g., Röper K et al. The ‘spectraplakins’: cytoskeletal giants with characteristics of both spectrin and plakin families. J Cell Sci. 2002 Nov. 15; 115 (Pt 22): 4215-25. Members of the spectrin Superfamily include but not limited to, alpha-actinin (for example, alpha-actin-1, see, e.g., UniProtKB—P12814 and on the world wide web at genecards.org/cgi-bin/carddisp.pl?gene=ACTN1; alpha-actin-2, see, e.g., UniProtKB—P35609 and on the world wide web at genecards.org/cgi-bin/carddisp.pl?gene=ACTN2; alpha-actin-3, see, e.g., UniProtKB—Q08043 and on the world wide web at genecards.org/cgi-bin/carddisp.pl?gene=ACTN3; and alpha-actin-4, see, e.g., UniProtKB—O43707 and on the world wide web at genecards.org/cgi-bin/carddisp.pl?gene=ACTN4, each of which is incorporated herein by its entirety), alpha-spectrin (for example, Spectrin alpha chain, erythrocytic 1, see, e.g., UniProtKB—P02549 and on the world wide web at genecards.org/cgi-bin/carddisp.pl?gene=SPTA1; and Spectrin alpha chain, non-erythrocytic 1, see, e.g., UniProtKB—Q13813 and on the world wide web at genecards.org/cgi-bin/carddisp.pl?gene=SPTAN1, each of which is incorporated herein by its entirety), beta-spectrin (for example, Spectrin beta chain, erythrocytic, see, e.g., UniProtKB—P11277 and on the world wide web at genecards.org/cgi-bin/carddisp.pl?gene=SPTB; and Spectrin beta chain, non-erythrocytic 1, see, e.g., UniProtKB—Q01082 and on the world wide web at genecards.org/cgi-bin/carddisp.pl?gene=SPTBN1, each of which is incorporated herein by its entirety), dystrophin, and utrophin.

Plakins are cytolinker proteins that associate with cytoskeletal elements and junctional complexes. See, e.g., Leung Cl et al. Plakins: a family of versatile cytolinker proteins. Trends Cell Biol. 2002 January; 12 (1): 37-45. Seven plakin family members have been identified: desmoplakin (UniProtKB—P15924 and on the world wide web at genecards.org/cgi-bin/carddisp.pl?gene=DSP, which, including the sequences listed therein, are enclosed herein by their entireties), plectin (UniProtKB—P15924 and on the world wide web at genecards.org/cgi-bin/carddisp.pl?gene=PLEC, which, including the sequences listed therein, are enclosed herein by their entireties), bullous pemphigoid antigen 1 (BPAG1, Dystonin) (UniProtKB—Q03001 and on the world wide web at genecards.org/cgi-bin/carddisp.pl?gene=DST, which, including the sequences listed therein, are enclosed herein by their entireties), microtubule-actin crosslinking factor (MACF) (UniProtKB—Q9UPN3 and on the world wide web at genecards.org/cgi-bin/carddisp.pl?gene=MACF1, which, including the sequences listed therein, are enclosed herein by their entireties), envoplakin (UniProtKB—Q92817 and on the world wide web at genecards.org/cgi-bin/carddisp.pl?gene=EVPL, which, including the sequences listed therein, are enclosed herein by their entireties), periplakin (UniProtKB—060437 and on the world wide web at genecards.org/cgi-bin/carddisp.pl?gene=PPL, which, including the sequences listed therein, are enclosed herein by their entireties) and epiplakin (UniProtKB—P58107 and on the world wide web at genecards.org/cgi-bin/carddisp.pl?gene=EPPK1, which, including the sequences listed therein, are enclosed herein by their entireties). This family of proteins is defined by the presence of a plakin domain and/or a plakin repeat domain (PRD). In addition to these two domains, plakins also harbor other domains that are common in some but not all members: the actin-binding domain (ABD), coiled-coil rod, spectrin-repeat-containing rod and microtubule-binding domain.

Spectraplakins belong within both the spectrin and plakin superfamilies and are exceptionally long, intracellular proteins that have the rare ability to bind to all three cytoskeletal elements: actin, microtubules, and intermediate filaments. Spectraplakins are critically important for tissue integrity and function, operating with single cytoskeleton elements as well as coordinating these elements. See, e.g., Röper K et al. as cited above and Huelsmann S et al, Spectraplakins. Curr Biol. 2014 Apr. 14; 24 (8): R307-8. doi: 10.1016/j.cub.2014.02.003. Members of spectraplakins include but not limited to BPAG1 and MACF as described above. Dystrophin anchors the extracellular matrix to the cytoskeleton via F-actin, and is a ligand for dystroglycan. Component of the dystrophin-associated glycoprotein complex accumulates at the neuromuscular junction (NMJ) and at a variety of synapses in the peripheral, and central nervous systems and has a structural function in stabilizing the sarcolemma. It is also implicated in signaling events and synaptic transmission. See, e.g., on the world wide web at uniprot.org/uniprot/P11532 and on the world wide web at genecards.org/cgi-bin/carddisp.pl?gene=DMD&keywords=dystrophin. There are 10 isoforms of dystrophin. Isoform 4 is considered as the canonical sequence and has an amino acid sequence with UniProtKB identifier: P11532-1, which is incorporated herein. The other isoforms including isoform 1 with UniProtKB identifier: P11532-2, isoform 2 with UniProtKB identifier: P11532-3, isoform 3 with UniProtKB identifier: P11532-4, isoform 5 with UniProtKB identifier: P11532-5, isoform 6 with UniProtKB identifier: P11532-6, isoform 7 with UniProtKB identifier: P11532-7, isoform 8 with UniProtKB identifier: P11532-8, isoform 9 with UniProtKB identifier: P11532-9, and isoform 10 with UniProtKB identifier: P11532-10, sequence of each of the isoform is incorporated herein. The term “full-length dystrophin” as used herein may refer to any dystrophin isoform. In certain embodiments, the term “full-length dystrophin” refers to the isoform 4 (Dp427). Homologs of dystrophin have been identified in a variety of organisms, including mouse (UniProt P11531), rat (UniProt P11530), and dog (UniProt 097592). Possible nucleic acid sequence encoding Dp427 or any other isoform or homolog of dystrophin is available publicly, See, e.g., NCBI Reference Sequences: NM_000109.3, NM_004006.2, NM_004009.3, NM_004010.3, NM_004011.3, NM_004012.3, NM_004013.2, NM_004014.2, NM_004015.2, NM_004016.2 NM_004017.2, NM_004018.2, NM_004019.2, NM_004020.3, NM_004021.2, NM_004022.2, NM_004023.2, NM_004007.2, XM_006724468.2, XM_006724469.3, XM_006724470.3, XM_006724473.2, XM_006724474.3, XM_006724475.2, XM_011545467.1, XM_011545468.2, XM_011545469.1, XM_017029328.1, XM_017029329.1, XM_017029330.1, and XM_017029331.1, each of which is incorporated herein. Additional sequence encoding Dp427 or any other isoform or homolog of dystrophin may be generated via tools for reverse-translation, e.g., on the world wide web at ebi.ac.uk/Tools/st/, on the world wide web at ebi.ac.uk/Tools/st/emboss_transeq/, on the world wide web at ebi.ac.uk/Tools/st/emboss_sixpack/, on the world wide web at ebi.ac.uk/Tools/st/emboss_backtranseq/, and on the world wide web at ebi.ac.uk/Tools/st/emboss_backtranambig/. Furthermore, the coding sequences might be codon-optimized for expression in a subject, e.g., human, mice, rat or dog.

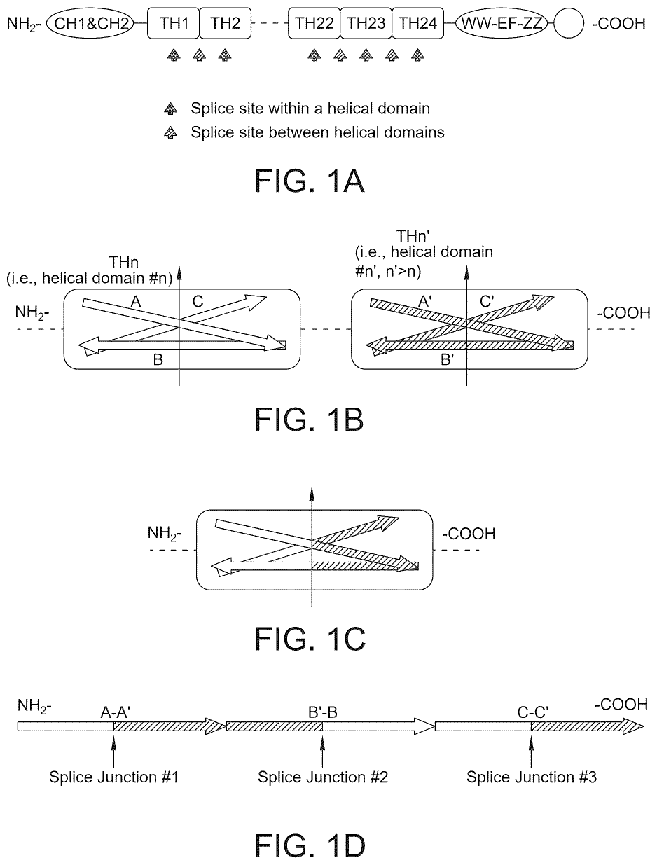

Dp427 is 3685 amino acids in length. Also, see, e.g., U.S. Pat. No. 7,892,824B2, and GenBank: AAA53189.1. The N-terminal 240 amino acids of Dp427 fold into two calponin homology domains (CH1&2) that have the capacity for high-affinity binding to cytoskeletal actin filaments. The central region from approximately amino acid 340 to 3040 is composed of 24 tandemly linked domains identifiable by Hidden Markov Modelling as triple helices (TH1-24) with measurable structural homology to crystalized repeats of the rod-like proteins spectrin and alpha-actinin. The region from 3057 to 3352 encompasses a modular, multi-domain globular region (WW-EF-ZZ) with high affinity for the membrane spanning complex of proteins centered around beta-dystroglycan. The extreme C-terminal region from 3353 to 3685 has seemingly expendable high-affinity binding domains for the proteins dystrobrevin and syntrophin. Dp427 has been modelled as a rod-like protein at the cortex of striated myocytes, bound to the outermost rim of cytoskeletal F-actin by the N-terminal calponin homology domains, and to the membrane spanning members of the DGC by the C-terminal WW-EF-ZZ domains. The central rod domain is composed of 24 domains with low level homology to the triple helical repeats hinges 1-4. Internal deletions of the spectrin-like repeats are generally associated with a slower rate of disease progression, in the allelic disease Becker MD (BMD). A central challenge for gene therapy for DMD is to safely, effectively, and durably substitute for Dp427 in the majority of skeletal and cardiac myocytes. Ideally this substitution would match the functionality of Dp427; there is concern that proteins substantially smaller than 427 kd might merely convert DMD to a severe BMD phenotype. An illustration of Dp427 can be found in FIG. 1A. Hidden Markov Modelling can be performed via conventional methods while parameters thereof may be adjusted by one of skill in the art. See., e.g., en.wikipedia.org/wiki/Hidden_Markov_model. Deletions of 19-20 contiguous TH domains and the region from 3353-3685 yield “AAV-sized” miniaturized dystrophins in the sense that synthetic coding sequences for these recombinant proteins are within the cloning capacity of AAV vectors. All such recombinant proteins share rod-like domains ⅕^{th} to ⅙^{th} of the length of the rod in Dp427, raising concerns that the shortening undermines the recombinant proteins' ability to “absorb” as much “shock” as the 24-repeat full-length protein.

Utrophin is a substantial homology to dystrophin, with significant divergence occurring in the rod domain, where utrophin lacks repeats 15 and 19 and two hinge regions (See e.g., Love et al., Nature 339:55 [1989]; Winder et al., FEBS Lett., 369:27 [1995]; on the world wide web at uniprot.org/uniprot/P46939; and on the world wide web at genecards.org/cgi-bin/carddisp.pl?gene=UTRN&keywords=Utrophin). Four isoforms of utrophin were discovered. Isoform 1 is considered as the canonical sequence and has an amino acid sequence with UniProt Identifier P46939-1, which is incorporated herein. Other isoforms include Isoform 2 with UniProt Identifier P46939-2; isoform Up71 with UniProt Identifier P46939-3; and isoform Up140 with UniProt Identifier P46939-4. Full-length utrophin may refer to any utrophin isoform. In certain embodiment, the full-length utrophin refers to utrophin isoform 1, which contains 22 spectrin-like repeats (SR1 to SR22, or TH1 to TH22) and two hinge regions. Homologs of utrophin have been identified in a variety of organisms, including mouse (Genbank accession number Y12229 and UniProt E9Q6R7), rat (Genbank accession number AJ002967 and UniProt G3V7L1), and dog (GenBank accession number NW-139836). The nucleic acid sequence of these or additional homologs can be compared to the nucleic acid sequence of human utrophin using any suitable methods. Nucleic acid sequence encoding utrophin isoform 1 or any other isoform or any homolog is available. See, e.g., NCBI Reference Sequence: NM_007124.2, XM_005267127.4, XM_005267130.2, XM_005267133.2, XM_006715560.3, XM_011536101.2, XM_011536102.2, XM_011536106.2, XM_011536107.2, XM_011536109.2, XM_017011243.1, XM_017011244.1, XM_017011245.1; Genbank accession number X69086 and GenBank accession number AL357149, each of which is incorporated herein. Additional sequence encoding utrophin isoform 1 or any other isoform or any homolog may be generated via tools for reverse-translation, e.g., on the world wide web at ebi.ac.uk/Tools/st/, on the world wide web at ebi.ac.uk/Tools/st/emboss_transeq/, on the world wide web at ebi.ac.uk/Tools/st/emboss_sixpack/, on the world wide web at ebi.ac.uk/Tools/st/emboss_backtranseq/, and on the world wide web at ebi.ac.uk/Tools/st/emboss_backtranambig/. Furthermore, the coding sequences might be codon optimized for expression in a subject, e.g., human, mice, rat or dog.

In one aspect, provided herein is a dystrophin superfamily triple splice mutant protein. In one embodiment, the triple splice mutant protein comprises an internal deletion of multiple helical repeats, and a hybrid helical domain formed by joining portions of helical repeats of the full-length dystrophin superfamily protein. In certain embodiments, the dystrophin superfamily triple splice mutant protein has a first helix with an N-terminal portion of a helix A fused to a C-terminal portion of a helix A′, a second helix comprising an N-terminal portion of a helix B′ fused to a C-terminal portion of a helix B, and a third helix comprising an N-terminal portion of a helix C fused to a C-terminal portion of helix C′, wherein helices A, B, and C are present in a first triple helical repeat and helices A′, B′, and C′ are present in a second triple helical domain in a native dystrophin superfamily protein. In certain, embodiments, the first and second triple helical domains are non-adjacent and, accordingly, provide a mutant dystrophin superfamily protein having a hybrid triple helical domain and a deletion of 1 or more triple helical domains present in the native dystrophin superfamily protein. Thus, the total number of the helical repeats in the triple splice mutant protein is selected from any integer from 3 to 1 less than the helical repeat number of the full-length dystrophin superfamily protein, for example, 4, 5, 6, 7, 8, 9, 10, 11, 12, 13, 14, 15, 16, 17, 18, 19, 20, 21, 22, and 23. In certain embodiments, provided herein are mutant dystrophin proteins that have deletions in at least helical repeat 3 to helical repeat 21 in the full-length dystrophin. In yet another embodiment, the mutant protein has a deletion in at least helical repeat 3 to 23 in the full-length dystrophin. In yet a further embodiment, the mutant protein has a deletion in at least helical repeat 2 to helical repeat 19 in the full-length dystrophin. In certain embodiments, the mutant utrophin proteins are provided having a deletion in at least helical repeat 3 to helical repeat 10 of full-length dystrophin. In a further embodiment, the mutant utrophin has a deletion in at least helical repeat 2 to helical repeat 17 of full length utrophin.

As used herein, the terms “triple helical domain”, “triple helix domain”, “triple helical repeat”, “triple helix repeat”, and “TH” are interchangeable and refers to the rod-like and spectrin-like repeat of a dystrophin superfamily protein consisting of three α-helices, i.e., two similarly (Helices A and C) and one oppositely (Helix B) directed α-helices, joined by nonhelical linkers. Those repeats can be identified by Hidden Markov Modelling. Examples of such repeats can be found in FIG. 2A to FIG. 2E. The hybrid triple helical repeat is formed by two helical repeats spliced on the plane that bisects the helical repeat perpendicular to its long axis. Such planes are further discussed below and exemplified in the Examples as well as in FIG. 2F. An illustration of forming such hybrid triple helical repeat is provided in FIG. 1 with an illustration of a hybrid triple helix domain (also noted herein as a hybrid triple helical repeat) presented in FIG. 1C and FIG. 1D. As used herein, a “splice junction” indicates a position in a nucleic acid sequence, an amino acid sequence, or a protein with secondary, tertiary or quaternary structure, where the internal deletion starts or ends in the full-length dystrophin superfamily protein or in either of the two triple helical repeats forming the hybrid triple helix domain; or where the sequences, corresponding sequences of which in the full-length protein are not immediately adjacent to each other, but are joined in the hybrid triple helix domain or in the dystrophin superfamily triple splice mutant protein. The term “immediately adjacent” means that two sequences, domains or repeats, are not separated by any other sequence, domains or repeats respectively. The term “join,” “re-join,” or any grammatical variations thereof indicates two sequences, domains or repeats become immediately adjacent. The term “form” or any grammatical variation thereof refers to splicing or joining sequences. The correspondence of sequences or positions in sequences may be determined by a sequence alignment or the Hidden Markov Model (HMM).

As used herein, an “N-terminal portion” refers to the amino acid sequence at the amino-terminal side of the splice junction for a selected helix. In certain embodiments, the “N-terminal portion” of a selected helix refers to the full-length amino acid sequence from the initial Met through to the last amino acid sequence prior to (on the N-terminal side) of the splice junction. In certain embodiments, there may be amino acid substitutions, deletions, truncations and/or insertions in the N-terminal portion. In certain embodiments, such substitutions are conservative amino acid changes. In certain embodiments, a deletion, truncation or insertion is from one to five amino acids in length which does not affect the folding of the helix.

The term “a “C-terminal portion” refers to the amino acid sequence at the carboxy-terminal side of the splice junction for a selected helix. In certain embodiments, the “C-terminal portion” of a selected helix refers to the full-length amino acid sequence from the first amino acid sequence after (on the C-terminal side) of the splice junction. In certain embodiments, there may be amino acid substitutions, deletions, truncations and/or insertions in the N-terminal portion. In certain embodiments, such substitutions are conservative amino acid changes. In certain embodiments, a deletion, truncation or insertion is from one to five amino acids in length which does not affect the folding of the helix.

For example, in certain embodiments, the triple mutant hybrid helical domains havcrosse three splice helices that are formed by joining segments of non-adjacent helical domains, wherein each helix comprises an N-terminal portion and a C-terminal portion of a helix in a helical repeat of a native dystrophin superfamily protein. Because the triple helical domains that are joined to form the mutant junction each have parallel A and C helices and an anti-parallel B helix, the N-terminal portions of the A and C helices in the triple helix mutant are from the same triple helical repeat in the native dystrophin superfamily protein, while the C-terminal portions of these helices are from another triple helical repeat in the native dystrophin superfamily protein. As result of the positioning of the junction to form the triple helical mutant domain, the N-terminal portions and C-terminal portions may be of varied lengths, but together form a helix of the mutant triple helix domain.

An ordinal number, such as “first,” “second,” “third,” “fourth,” or the term “additional” are used throughout this specification as reference terms to distinguish between various forms and components of the compositions and methods. Unless specified, if an ordinal number is used for indicating a TH, an amino acid sequence, or a nucleic acid sequence, such number is counted from N-terminal to C-terminal in an amino acid sequence or a protein, or from 5′ to 3′ in a nucleic acid sequence.

As used herein, a protein repeat followed by a number refers to the repeat number among all repeats in a reference protein or a reference amino acid sequence counting from the N-terminal unless particularly specified. For example, the triple helix domain 2 refers to TH2 illustrated by FIG. 1A. When the repeat and the reference sequence are polynucleotides, the number of the repeats is counted from the 5′ end to the 3′end unless particularly specified.

In certain embodiments, the dystrophin superfamily triple splice mutant protein comprises N-terminal helical repeat(s), a hybrid triple helical repeat, and C-terminal helical repeat(s). In certain embodiments, the dystrophin superfamily triple splice mutant protein comprises a hybrid triple helical repeat and C-terminal helical repeats. The total number of the helical repeat(s) in the triple splice mutant protein is selected from any integer from 3 to 1 less than the helical repeat number of the full-length dystrophin superfamily protein, for example, 4, 5, 6, 7, 8, 9, 10, 11, 12, 13, 14, 15, 16, 17, 18, 19, 20, 21, 22, and 23. In certain embodiments, sequence(s) in the full-length dystrophin superfamily protein, which corresponds to the N-terminal helical repeat(s) in the mutant protein, is immediately adjacent to sequence corresponding to the first of the two helical repeats which forms the hybrid triple helical repeat. In certain embodiments, sequence(s) in the full-length dystrophin superfamily protein, which corresponds to the C-terminal helical repeat(s) in the mutant protein, is immediately adjacent to sequence corresponding to the second of the two helical repeats which forms the hybrid triple helical repeat. In a further embodiment, the N-terminal helical repeat(s) may comprise TH1, TH2, TH3, TH4, TH5, TH6, TH7, TH8, TH9, TH10, TH11, TH12, TH13, TH14, TH15, TH16, TH17, TH18, and TH19. In yet a further embodiment, the C-terminal helical repeat(s) may comprise TH1, TH2, TH3, TH4, TH5, TH6, TH7, TH8, TH9, TH10, TH11, TH12, TH13, TH14, TH15, TH16, TH17, TH18, and TH19, each TH number of which is counted from the C-terminal. Additionally, there might be only one helical repeat in the mutant protein wherein the helical repeat is the hybrid triple helix domain formed by helical domain 1 and the last helical domain of the full-length dystrophin superfamily protein. Furthermore, there might be two helical repeats in the mutant protein. Such two-TH mutant protein may comprise helical repeat 1 of the full-length protein, and one hybrid triple helix domain formed by helical repeat 2 and the last helical repeat of the full-length protein. In another embodiment, the two-TH mutant protein may comprise the last helical repeat in the full-length protein, and one hybrid triple helix domain formed by helical repeat 1 and the second from the last helical repeat of the full-length protein. As used herein, the terms “dystrophin superfamily triple splice mutant protein”, “triple splice mutant protein” and “mutant protein” are used interchangeably. Also, except at the splice junction(s) in the hybrid triple helical repeat, the repeats and sequences in the mutant protein are immediately adjacent to the same repeats and sequences respectively as they are in the full-length dystrophin superfamily protein.

In certain embodiments, the dystrophin superfamily triple splice mutant protein might be truncated at the C-terminal, for example, for any integer of 1 to 500 of amino acids. Such truncation does not comprise any triple helical repeat. In certain embodiments, such truncation may occur at a position corresponding to a beginning or an end of an exon of the full-length dystrophin superfamily protein.

Also provided herein is a nucleic acid sequence encoding the dystrophin superfamily triple splice mutant protein. Such coding sequence may be generated via tools for reverse-translation. Furthermore, the coding sequences might be codon optimized for expression in a subject, e.g., human, mice, rat or dog.

In one embodiment, the dystrophin superfamily mutant protein is a triple splice mutant dystrophin. In a further embodiment, the triple splice mutant dystrophin comprises a deletion in at least helical repeat 3 to helical repeat 21 of the full-length dystrophin. In another embodiment, the triple splice mutant dystrophin comprises a deletion in at least helical repeat 3 to helical repeat 23 of the full-length dystrophin. In yet another embodiment, the N-terminal helical repeat(s) of the mutant dystrophin comprises helical repeat 1 in the full-length dystrophin. The C-terminal helical repeat(s) of the mutant protein comprises helical repeat 23 and helical repeat 24 in the full-length dystrophin. In a further embodiment, the N-terminal helical repeat of the mutant protein consists of helical repeat 1 in the full-length dystrophin. The C-terminal helical repeats of the mutant protein consist of helical repeat 23 and helical repeat 24 in the full-length dystrophin. The first of the two helical repeats which forms the hybrid triple helical repeat is helical repeat 2 in the full-length dystrophin. The second of the two helical repeats which forms the hybrid triple helical repeat is helical repeat 22 in the full-length dystrophin. In yet another embodiment, the triple splice mutant dystrophin comprises a deletion in at least helical repeat 2 to helical repeat 19 of the full-length dystrophin protein. The C-terminal helical repeats comprise helical repeats 21, 22, 23, and 24 in the full-length dystrophin. The first of the two helical repeats which forms the hybrid triple helical repeat is helical repeat 1 in the full-length dystrophin, and the second of the two helical repeats which forms the hybrid triple helical repeat is helical repeat 19 in the full-length dystrophin. In certain embodiments, the triple splice mutant dystrophin may further comprise amino acid (aa) about 1 to about aa 338 of dystrophin isoform 4 at the N-terminal. In certain embodiments, the triple splice mutant dystrophin may further comprise about aa 3041 to about aa 3352, about aa 3041 to about aa 3054, about aa 3041 to about aa 3056, about aa 3041 to about aa 3057, about aa 3041 to about aa 3088, about aa 3041 to about aa 3408, or about aa 3041 to about aa 3685 of dystrophin isoform 4 at the C-terminal.

In certain embodiments, there may be a further truncation of these C-terminal non-TH sequence of a triple splice mutant dystrophin. As used herein, a truncation refers to a deletion of consecutive amino acids starting from the C-terminal end. In certain embodiments, such truncation may occur at a position corresponding to a beginning or an end of an exon of the full-length dystrophin. In certain embodiment, the truncation may be 1, 2, 3, 4, 5, about 10, about 15, about 20, about 30, about 40, about 50, about 60, about 70, about 100, about 150, about 200, about 250, about 300, about 400, about 500, or about 600 aa in length. Also provided herein is a nucleic acid sequence encoding the triple splice mutant dystrophin. Such coding sequence may be generated via tools for reverse-translation. Furthermore, the coding sequences might be codon optimized for expression in a subject, e.g., human, mice, rat or dog.

In one embodiment, the triple mutant dystrophin is a nano-dystrophin (also noted as n-dystrophin) having the amino acid sequence of SEQ ID NO: 1. In yet a further embodiment, provided is a sequence encoding the triple splice mutant dystrophin having a nucleic acid sequence of SEQ ID NO: 2. In one embodiment, the nucleic acid sequence encoding SEQ ID NO: 1 is codon-optimized for expression in a subject. In a further embodiment, the nucleic acid sequence encoding SEQ ID NO: 1 is codon-optimized for expression in human. Conventional tools for codon optimization are available publicly or commercially to one of skill in the art. See, e.g., Fuglsang A (Codon optimizer: a freeware tool for codon optimization. Protein Expr Purif. 2003 October; 31 (2): 247-9,) on the world wide web at genscript.com/codon-opt.html, on the world wide web at thermofisher.com/us/en/home/life-science/cloning/gene-synthesis/geneart-gene-synthesis/geneoptimizer.html, and on the world wide web at idtdna.com/CodonOpt.

In yet another embodiment, the triple mutant dystrophin is a nano-dystrophin having the amino acid sequence of SEQ ID NO: 13, 14, 15, 16, 17, or 18. In yet a further embodiment, provided herein are nucleic acid sequences encoding the triple splice mutant dystrophin having an amino acid sequence of SEQ ID NO: 13, 14, 15, 16, 17, or 18. In certain embodiments, the nucleic acid sequence encoding SEQ ID NO: SEQ ID NO: 13, 14, 15, 16, 17, or 18 is codon-optimized for expression in a subject. In a further embodiment, the nucleic acid sequence encoding SEQ ID NO: SEQ ID NO: 13, 14, 15, 16, 17, or 18 is codon-optimized for expression in human.

In yet another embodiment, the triple mutant dystrophin is a nano-dystrophin having the amino acid sequence of SEQ ID NO: 22.

The term “subject” as used herein means a male or female mammalian animal, including a human, a veterinary or farm animal, a domestic animal or pet, and animals normally used for clinical research. In one embodiment, the subject of these methods and compositions is a human. In one embodiment, the subject of these methods and compositions is a prenatal, a newborn, an infant, a toddler, a preschool, a grade-schooler, a teen, a young adult or an adult. A newborn human refers to a human with an age of 0 to 12 month; a human toddler is with an age of 1 to 3 years; a human preschool with an age of 3 to 5 years; a human grade-schooler with an age of 5 to 12 years; a human teen with 12 to 18 years old; a human young adult with an age of 18 to 21 years; while a human adult with an age beyond 18 years. A “healthy subject” refers to a subject without a disease. As used herein, the term “disease” may refer to DMD and/or BMD. In certain embodiments, the term “disease” may refer to another disease caused by an abnormal dystrophin superfamily protein. In certain embodiments, “disease” refers to von Willebrand's disease.

In one embodiment, the dystrophin superfamily mutant protein is a triple splice mutant utrophin and comprises a deletion in at least helical repeat 3 and helical repeat 19 of full-length utrophin. In one embodiment, the N-terminal helical repeat(s) of the mutant protein comprises helical repeat 1 in the full-length utrophin. The C-terminal helical repeat(s) of the mutant protein comprises helical repeat 21 and helical repeat 22 in the full-length utrophin. In a further embodiment, the N-terminal helical repeat of the mutant protein consists of helical repeat 1 in the full-length utrophin. The C-terminal helical repeats of the mutant protein consist of helical repeat 21 and helical repeat 22 in the full-length utrophin. The first of the two helical repeats which forms the hybrid triple helical repeat is helical repeat 2 in the full-length utrophin. The second of the two helical repeats which forms the hybrid triple helical repeat is helical repeat 20 in the full-length utrophin. In yet another embodiment, the dystrophin superfamily mutant protein is a triple splice mutant utrophin and include a deletion in at least helical repeat 2 to helical repeat 17 of full-length utrophin. The C-terminal helical repeats of the mutant protein include helical repeats 19, 20, 21, and 22 in the full-length utrophin. The first of the two helical repeats which forms the hybrid triple helical repeat is helical repeat 1 in the full-length utrophin, and the second of the two helical repeats which forms the hybrid triple helical repeat is helical repeat 18 in the full-length utrophin. In certain embodiments, the triple splice mutant utrophin may further comprise about aa 1 to about aa 311 of utrophin isoform 1 at the N-terminal. In certain embodiments, the triple splice mutant utrophin may further comprise about aa 2797 to about 2811, about aa 2797 to about 2845, about aa 2797 to about 3124, about aa 2797 to about 3134, about aa 2797 to about 3165, about aa 2797 to about 3168, or about aa 2797 to about 3433, of utrophin isoform 1 at the C-terminal. In certain embodiments, there may be a further truncation of these C-terminal non-TH sequence of a triple splice mutant utrophin. In certain embodiments, such truncation may occur at a position corresponding to a beginning or an end of an exon of the full-length utrophin. In certain embodiment, the truncation may be 1, 2, 3, 4, 5, about 10, about 15, about 20, about 30, about 40, about 50, about 60, about 70, about 100, about 150, about 200, about 250, about 300, about 400, about 500, or about 600 aa in length. Also provided herein is a nucleic acid sequence encoding the triple splice mutant utrophin. Such coding sequence may be generated via tools for reverse-translation. Furthermore, the coding sequences might be codon optimized for expression in a subject, e.g., human, mice, rat or dog.

In one embodiment, the triple mutant utrophin is a nano-utrophin (also noted as n-utrophin, or n-U with or without the dash mark) comprising the amino acid sequence of: SEQ ID NO: 3. In a further embodiment, provided herein is a sequence encoding the triple splice mutant utrophin and comprising a nucleic acid sequence of SEQ ID NO: 4. In certain embodiments, the triple mutant utrophin is a nano-utrophin (also noted as n-utrophin, or n-U with or without the dash mark) having the amino acid sequence of SEQ ID NO: 5. In a further embodiment, provided herein is a sequence encoding SEQ ID NO: 5 and having a nucleic acid sequence of SEQ ID NO: 6, or a nucleic acid sequence about 95% to about 99% identical to SEQ ID NO: 6. In certain embodiments, the triple mutant utrophin is a nano-utrophin having the amino acid sequence of SEQ ID NO: 7. In a further embodiment, provided herein is a sequence encoding SEQ ID NO: 7, and having a nucleic acid sequence of SEQ ID NO: 8 or a nucleic acid sequence about 95% to about 99% identical to SEQ ID NO: 8. In one embodiment, the nucleic acid sequence encoding SEQ ID NOs: 3, 5, or 7 is codon-optimized for expression in a subject. In certain embodiments, the triple mutant utrophin is a nano-utrophin having the amino acid sequence of SEQ ID NO: 20. In a further embodiment, provided herein is a sequence encoding SEQ ID NO: 20, and having a nucleic acid sequence of SEQ ID NO: 19 or a nucleic acid sequence about 95% to about 99% identical to SEQ ID NO: 19. In one embodiment, the nucleic acid sequence encoding SEQ ID NOs: 3, 5, 7, or 20 is codon-optimized for expression in a subject. In a further embodiment, the nucleic acid sequence encoding SEQ ID NOs: 3, 5, 7, or 20 is codon-optimized for expression in human. In yet another embodiment, the triple mutant utrophin is a nano-utrophin comprising the amino acid sequence of SEQ ID NO: 21.

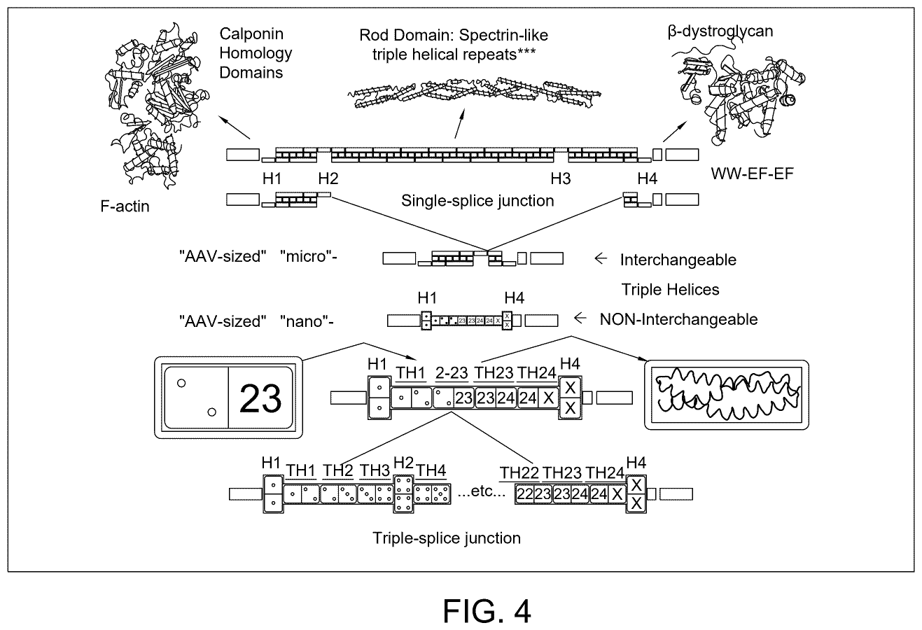

In certain embodiments, this disclosure includes the amino acid and all encoding synthetic nucleic acid sequences for both human nano-utrophin and human nano-dystrophin featuring “triple-spliced”. In certain embodiment, hybrid triple helices may join the middle of triple helical repeat 2 to that of the third-from-the-last triple helical repeat domain (#20 of 22 in utrophin, #22 of 24 in dystrophin), giving a total of four repetitive domains in both of the recombinant proteins as described herein and illustrated as features of SEQ ID NOs: 1 and 3. These amino acid sequences define the recombinant proteins of greatest strength that can be encoded within the coding capacity of a single AAV vector genome. See FIG. 4 and Examples 2 and 3.

Little was known of the mechanobiology of dystrophin, but indirect studies of the protein's physiological role suggest that the rod domain of the protein may be loaded longitudinally during muscle contraction. The examples presented herein provides the first direct evidence for this. In comparisons between juvenile and skeletally mature mdx (dystrophin null) mice expressing micro-utrophin, western blot analysis with antibodies directed against the N-terminus of the recombinant protein reveals evidence for disruption of the rod at the position of the single splice junction as the processes of muscle maturation and myosin isoform switching increase the mechanical loading across the muscle membrane. This strongly supports the hypothesis that the strength of the rod domain is compromised at the exact position of the single splice junction. The design principle underlying the development of nano-utrophin and nano-dystophin compensates for this previous shortcoming by eliminating the juxtaposition of incompatible subdomains of the rod.

### A. Splice Junctions in the Helices

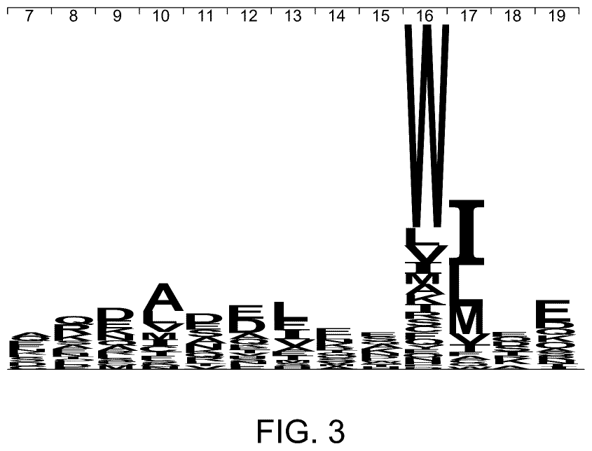

The hybrid triple helical repeat is formed by two helical repeats spliced on the plane that bisects the helical repeat perpendicular to its long axis as indicated in FIG. 1A to FIG. 1D and FIG. 2F. The choice of splice junction in the antiparallel “B” helix is derived by indirect means. Only one X-ray crystal structure has been determined for a single repeat of a dystrophin triple helix, TH1 (i.e. no structures to date for TH2-24). The adjacent triple helices of dystrophin overlap more-so than do those of spectrin and alpha-actinin, thereby stabilizing the helices during longitudinal load-bearing, may partially explain the dearth of structural information as there may be difficulty in crystalizing sub-regions of the rod. Nonetheless, the conservation of tryptophan residues in the center of the hydrophobic core provides “anchor points” for two of the three splices, those in the “A” and “C” helices. Note for instance the prominence of the W at position 16 in the HMM logo shown in FIG. 3 (Wheeler et al., BMC Bioinformatics 2014). All crystal structures were analyzed for triple helical repeats containing the two interacting tryptophan residues and used the HMMscan analysis on the HMMer web portal to define the probability that individual positions within the “B” helix would correspond to the cross-sectional plane bisecting the tryptophans (i.e., the plane that bisects the helical repeat perpendicular to its long axis).

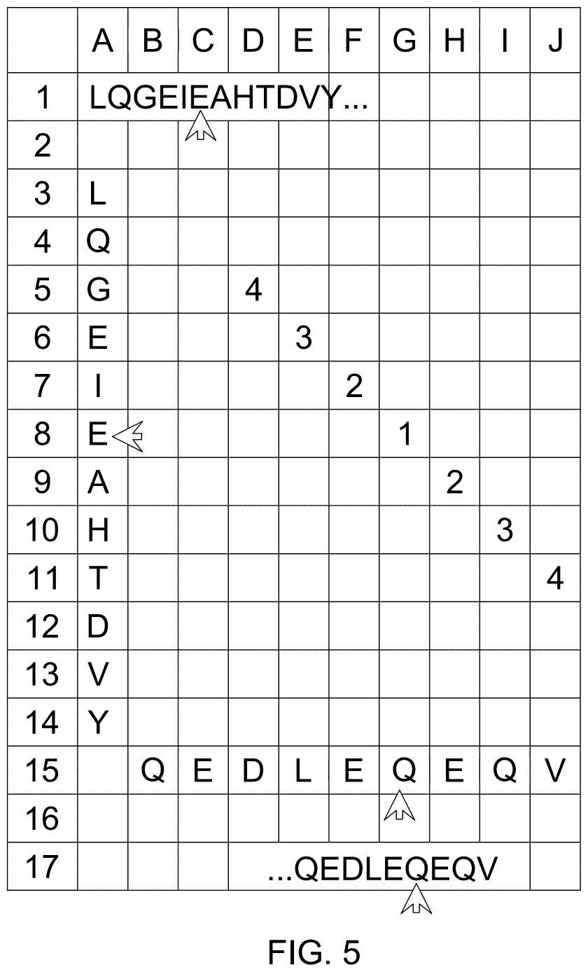

LQGEIEAHTDVY (N-terminal to C-terminal, an amino acid sequence in the full length dystrophin TH22) . . . . QEDLEQEQV (N-terminal to C-terminal, an amino acid sequence in the full length dystrophin TH2) is the sequence surrounding the splice junction in helix B (helix 2, the second helice of the three helices in a TH) of the two TH of the full-length dystrophin forming the hybrid TH in the nano-dystrophin. The underlined letters E and Q indicate the splice junction in Helices B while both of E and Q are preserved in the resultant hybrid TH as illustrated in SEQ ID NO: 1. However, it would be understood by one of skill in the art that the triple splice mutant dystrophin used herein may have a splice juction in helix B other than between EQ as indicated as a feature of SEQ ID NO: 1. Such splice juction may be at the N-terminal or C-terminal of any amino acid in Helix B as indicated in LQGEIEAHTDVY . . . QEDLEQEQV as well as FIG. 5 and the corresponding amino acid sequences of SEQ ID NOs: 13-18, or an other corresponding positon of a helix B in another TH. The correspondence of positions in sequences may be determined by amino acid sequence alignment between any two or more dystrophin TH or the Hidden Markov Model (HMM).

Similarly, the triple splice mutant utrophin used herein may have a splice juction in helix B between HQ as indicated as a feature of SEQ ID NO: 3, or may be at the N-terminal or C-terminal of any amino acid in Helix B in AEIDAHNDIFKS (N-terminal to C-terminal, an amino acid sequence in the full length utrophin TH20) . . . . DL EAEQVKV (N-terminal to C-terminal, an amino acid sequence in the full length utrophin TH2), or an other corresponding position of a helix B in another utrophin TH. The correspondence of positions in sequences may be determined by amino acid sequence alignment between any two or more utrophin TH or the Hidden Markov Model (HMM).

In certain embodiments, the splice junction in Helices A or C (which are helix 1, i.e., the first helix, and helix 3, i.e., the third helix, of the three helices in a triple helical repeat) may be at Tryptophan (W) at the core of the Hidden Markov Model (HMM) and all crystal structures for proteins in this superfamily. In a further embodiment, the splice junction in Helices A or C may be at a position which is 1 amino acid, 2 amino acids, 3 amino acids, 4 amino acids, or 5 amino acids from the W(s) to the C-terminal side or to the N-terminal side of the protein.

### B. Human Nano-Utrophin Sequence

The larger capital letters in sequences below designate the portion identical to N-terminal region of full-length human utrophin. The smaller capital letters designate the region identical to C-terminal region of full length human utrophin. W's in italics correspond to tryptophan residues in the “A” and “C” helices at the core of the Hidden Markov Model (HMM) and all crystal structures for proteins in this superfamily, HQ in italics correspond to the positions within the superfamily HMM for the “B” helix that flank the hypothetical plane of transection as depicted in the FIG. 2F. The anticipated secondary and tertiary structures of the folded protein correspond to the hybrid TH depicted in FIG. 4.

(SEQ ID NO: 3)
MAKYGEHEASPDNGQNEFSDIIKSRSDEHNDVQKKTFTKWINARFSKSGK
PPINDMFTDLKDGRKLLDLLEGLTGTSLPKERGSTRVHALNNVNRVLQVL
HQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWD
KVVKMSPIERLEHAFSKAQTYLGIEKLLDPEDVAVQLPDKKSIIMYLTSL
FEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFA
THEAFMMELTAHQSSVGSVLQAGNQLITQGTLSDEEEFEIQEQMTLLNAR
WEALRVESMDRQSRLHDVLMELQKKQLQQLSAWIQEAETTVNVLVDAS
HRENALQDSILARELKQQMQDIQAEIDAHQVKVNSLTHMVVIVDENSG
ESATAILEDQLQKLGERWNDLKAKSASIRAHLEASAEKWNRLLMSLEEL
IKWLNMKDEELKKQMPIGGDVPALQLQYDHCKALRRELKEKEYSVLNAVD
QARVFLADQPIEAPEEPRRNLQSKTELTPEERAQKIAKAMRKQSSEVKEK
WESLNAVTSNWQKQVDKALEKLRDLQGAMDDLDADMKEAESVRNGWKPVG
DLLIDSLQDHIEKIMAFREEIAPINFKVKTVNDLSSQLSPLDLHPSLKMS
RQLDDLNMRWKLLQVSVDDRLKQLQEAHRDFGPSSQHFLSTSVQLPWQRS
ISHNKVPYYINHQTQTTCWDHPKMTELFQSLADLNNVRFSAYRTAIKIRR
LQKALCLDLLELSTTNEIFKQHKLNQNDQLLSVPDVINCLTTTYDGLEQM
HKDLVNVPLCVDMCLNWLLNVYDTGRTGKIRVQSLKIGLMSLSKGLLEEK
YRYLFKEVAGPTEMCDQRQLGLLLHDAIQIPRQLGEVAAFGGSNIEPSVR
SCFQQNNNKPEISVKEFIDWMHLEPQSMVWLPVLHRVAAAETAKHQAKCN
ICKECPIVGFRYRSLKHFNYDVCQSCFFSGRTAKGHKLHYPMVEYCIPTT
SGE

In certain embodiments, the nano-utrophin provided herein comprises the following amino acid sequence, which includes a triple splice mutation joining repeats 2 and 20 in full-length utrophin:

(SEQ ID NO: 20)
MAKYGEHEASPDNGQNEFSDIIKSRSDEHNDVQKKTFTKWINARFSKSGK
PPINDMFTDLKDGRKLLDLLEGLTGTSLPKERGSTRVHALNNVNRVLQVL
HQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWD
KVVKMSPIERLEHAFSKAQTYLGIEKLLDPEDVAVQLPDKKSIIMYLTSL
FEVLPQQVTIDAIREVETLPRKYKKECEEEAINIQSTAPEEEHESPRAET
PSTVTEVDMDLDSYQIALEEVLTWLLSAEDTFQEQDDISDDVEEVKDQFA
THEAFMMELTAHQSSVGSVLQAGNQLITQGTLSDEEEFEIQEQMTLLNAR
WEALRVESMDRQSRLHDVLMELQKKQLQQLSAWIQEAETTVNVLVDASHR
ENALQDSILARELKQQMQDIQAEIDAHQVKVNSLTHMVVIVDENSGESAT
AILEDQLQKLGERWNDLKAKSASIRAHLEASAEKWNRLLMSLEELIKWLN
MKDEELKKQMPIGGDVPALQLQYDHCKALRRELKEKEYSVLNAVDQARVF
LADQPIEAPEEPRRNLQSKTELTPEERAQKIAKAMRKQSSEVKEKWESLN
AVTSNWQKQVDKALEKLRDLQGAMDDLDADMKEAESVRNGWKPVGDLLID
SLQDHIEKIMAFREEIAPINFKVKTVNDLSSQLSPLDLHPSLKMSRQLDD
LNMRWKLLQVSVDDRLKQLQEAHRDFGPSSQHFLSTSVQLPWQRSISHNK
VPYYINHQTQTTCWDHPKMTELFQSLADLNNVRFSAYRTAIKIRRLQKAL
CLDLLELSTTNEIFKQHKLNQNDQLLSVPDVINCLTTTYDGLEQMHKDLV
NVPLCVDMCLNWLLNVYDTGRTGKIRVQSLKIGLMSLSKGLLEEKYRYLF
KEVAGPTEMCDQRQLGLLLHDAIQIPRQLGEVAAFGGSNIEPSVRSCFQQ
NNNKPEISVKEFIDWMHLEPQSMVWLPVLHRVAAAETAKHQAKCNICKEC
PIVGFRYRSLKHFNYDVCQSCFFSGRTAKGHKLHYPMVEYCIPTTSGEDV
RDFTKVLKNKFRSKKYFAKHPRLGYLPVQTVLEGDNLET

The detailed design of nano-Utrophin to address structural constraints are described herein. Phylogenetic analysis suggests that the ancestral triple helical repeat existed in a protein orthologous to alpha-actinin, as this is the only protein in the proteomes of most single celled eukaryotes to match the spectrin consensus. Training sets including alpha-actinins, alpha- and beta-spectrins, and dystroplakins create HMMs for which the logos show exceptional conservation of the tryptophan residues. In available high resolution crystal structures, the position of the side chains and aromatic interaction is highly conserved, as is the structure of the third “B” alpha helix. In the sequences above, note the positions of the underlined W's and the B helix amino acids H and Q. The rearrangement or “splicing” of polypeptide sequences corresponding to these subdomains is depicted by different font size, with three sites of focal discontinuity corresponding to the plane of section illustrated in FIG. 2F, creating a 3-D hybrid between utrophin repeats 2 and 20. Also provided herein is a nano-utrophin having five spectrin-like triple-helical repeats, including a hybrid triple helical domain formed by splicing TH 1 and 18 in the full-length human utrophin protein. In certain embodiments, the nano-utrophin has the following sequence:

(SEQ ID NO: 21)
MAKYGEHEASPDNGQNEFSDIIKSRSDEHNDVQKKTFTKWINARFSKSGK
PPINDMFTDLKDGRKLLDLLEGLTGTSLPKERGSTRVHALNNVNRVLQVL
HQNNVELVNIGGTDIVDGNHKLTLGLLWSIILHWQVKDVMKDVMSDLQQT
NSEKILLSWVRQTTRPYSQVNVLNFTTSWTDGLAFNAVLHRHKPDLFSWD
KVVKMSPIERLEHAFSKAQTYLGIEKLLDPEDVAVQLPDKKSIIMYLTSL
FEVLPQQVTIDAIREVETLPRKYKKECEEEAINIQSTAPEEEHESPRAET
PSTVTEVDMDLDSYQIALEEVLTWLVLIDQMLKSNIVTVGDVEEINKTVS
RMKITKADLEQRQSSVGSVLQAGNQLITQGTLSDEEEFEIQEQMTLLNAR
WDGTQHGVELRQQQLEDMIIDSLQWDDHREETEELMRKYEARLYILQQAR
RDPLTKQISDNQILLQELGPGDGIVMAFDNVLQKLLEEYGSDDTRNVKET
TEYLKTSWINLKQSIADRQNALEAEWRTVQASRRDLENFLKWIQEAETTV
NVLVDASHRENALQDSILARELKQQMQDIQAEIDAHNDIFKSIDGNRQKM
VKALGNSEEATMLQHRLDDMNQRWNDLKAKSASIRAHLEASAEKWNRLLM
SLEELIKWLNMKDEELKKQMPIGGDVPALQLQYDHCKALRRELKEKEYSV
LNAVDQARVFLADQPIEAPEEPRRNLQSKTELTPEERAQKIAKAMRKQSS
EVKEKWESLNAVTSNWQKQVDKALEKLRDLQGAMDDLDADMKEAESVRNG
WKPVGDLLIDSLQDHIEKIMAFREEIAPINFKVKTVNDLSSQLSPLDLHP
SLKMSRQLDDLNMRWKLLQVSVDDRLKQLQEAHRDFGPSSQHFLSTSVQL
PWQRSISHNKVPYYINHQTQTTCWDHPKMTELFQSLADLNNVRFSAYRTA
IKIRRLQKALCLDLLELSTTNEIFKQHKLNQNDQLLSVPDVINCLTTTYD
GLEQMHKDLVNVPLCVDMCLNWLLNVYDTGRTGKIRVQSLKIGLMSLSKG
LLEEKYRYLFKEVAGPTEMCDQRQLGLLLHDAIQIPRQLGEVAAFGGSNI
EPSVRSCFQQNNNKPEISVKEFIDWMHLEPQSMVWLPVLHRVAAAETAKH
QAKCNICKECPIVGFRYRSLKHFNYDVCQSCFFSGRTAKGHKLHYPMVEY
CIPTTSGE

### C. Human Nano-Dystrophin Sequence:

The larger capital letters below designate portion identical to N-terminal region of full length human dystrophin. Smaller capital letters designate region identical to C-terminal region of full length human dystrophin. W's underlined correspond to tryptophan residues in the “A” and “C” helices at the core of the Hidden Markov Model (HMM) and all crystal structures for proteins in this superfamily. The EQ underlined corresponds to the positions within the superfamily HMM for the “B” helix that flank the hypothetical plane of transection as depicted in FIG. 2F. The anticipated secondary and tertiary structures of the folded protein correspond to the hybrid TH depicted in FIG. 4.

(SEQ ID NO: 1)
MLWWEEVEDCYEREDVQKKTFTKWVNAQFSKFGKQHIENLFSDLQDGRRL
LDLLEGLTGQKLPKEKGSTRVHALNNVNKALRVLQNNNVDLVNIGSTDIV
DGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAF
NIARYQLGIEKLLDPEDVDTTYPDKKSILMYITSLFQVLPQQVSIEAIQE
VEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEE
VLSWLLSAEDTLQAQGEISNDVEVVKDQFHTHEGYMMDLTAHQGRVGNIL
QLGSKLIGTGKLSEDEETEVQEQMNLLNSRWECLRVASMEKQSNLHRVLM
DLQNQKLKELNDWLTEAETTANVLQDATRKERLLEDSKGATKELMKQWQD
LQGEIEQEQVRVNSLTHMVVVVDESSGDHATAALEEQLKVLGDRWANICR
WSELRKKSLNIRSHLEASSDQWKRLHLSLQELLVWLQLKDDELSRQAPIG
GDFPAVQKQNDVHRAFKRELKTKEPVIMSTLETVRIFLTEQPLEGLEKLY
QEPRELPPEERAQNVTRLLRKQAEEVNTEWEKLNLHSADWQRKIDETLER
LQELQEATDELDLKLRQAEVIKGSWQPVGDLLIDSLQDHLEKVKALRGEI
APLKENVSHVNDLARQLTTLGIQLSPYNLSTLEDLNTRWKLLQVAVEDRV
RQLHEAHRDFGPASQHFLSTSVQGPWERAISPNKVPYYINHETQTTCWDH
PKMTELYQSLADLNNVRFSAYRTAMKLRRLQKALCLDLLSLSAACDALDQ
HNLKQNDQPMDILQIINCLTTIYDRLEQEHNNLVNVPLCVDMCLNWLLNV
YDTGRTGRIRVLSFKTGIISLCKAHLEDKYRYLFKQVASSTGFCDQRRLG
LLLHDSIQIPRQLGEVASFGGSNIEPSVRSCFQFANNKPEIEAALFLDWM
RLEPQSMVWLPVLHRVAAAETAKHQAKCNICKECPIIGFRYRSLKHFNYD
ICQSCFFSGRVAKGHKMHYPMVEYCTPTTSGE.

Also provided herein is a nano-dystrophin having five spectrin-like triple-helical repeats, including a hybrid triple helical domain formed by splicing TH 1 and 20 in the full-length human dystrophin. In certain embodiments, the nano-utrophin has the following sequence:

(SEQ ID NO: 22)
MLWWEEVEDCYEREDVQKKTFTKWVNAQFSKFGKQHIENLFSDLQDGRRL
LDLLEGLTGQKLPKEKGSTRVHALNNVNKALRVLQNNNVDLVNIGSTDIV
DGNHKLTLGLIWNIILHWQVKNVMKNIMAGLQQTNSEKILLSWVRQSTRN
YPQVNVINFTTSWSDGLALNALIHSHRPDLFDWNSVVCQQSATQRLEHAF
NIARYQLGIEKLLDPEDVDTTYPDKKSILMYITSLFQVLPQQVSIEAIQE
VEMLPRPPKVTKEEHFQLHHQMHYSQQITVSLAQGYERTSSPKPRFKSYA
YTQAAYVTTSDPTRSPFPSQHLEAPEDKSFGSSLMESEVNLDRYQTALEE
VLSWLSLLDQVIKSQRVMVGDLEDINEMIIKQKATMQDLEQRQGRVGNIL
QLGSKLIGTGKLSEDEETEVQEQMNLLNSRWDEVQEHLQNRRQQLNEMLK
DSTQWLEAKEEAEQVLGQARAKLESWKEGPYTVDAIQKKITETKQLAKDL
RQWQTNVDVANDLALKLLRDYSADDTRKVHMITENINASWRSIHKRVSER
EAALEETHRLLQQFPLDLEKFLAWLTEAETTANVLQDATRKERLLEDSKG
VKELMKQWQDLQGEIEAHTDVYHNLDENSQKILRSLEGSDDAVLLQRRLD
NMNFKWSELRKKSLNIRSHLEASSDQWKRLHLSLQELLVWLQLKDDELSR
QAPIGGDFPAVQKQNDVHRAFKRELKTKEPVIMSTLETVRIFLTEQPLEG
LEKLYQEPRELPPEERAQNVTRLLRKQAEEVNTEWEKLNLHSADWQRKID
ETLERLQELQEATDELDLKLRQAEVIKGSWQPVGDLLIDSLQDHLEKVKA
LRGEIAPLKENVSHVNDLARQLTTLGIQLSPYNLSTLEDLNTRWKLLQVA
VEDRVRQLHEAHRDFGPASQHFLSTSVQGPWERAISPNKVPYYINHETQT
TCWDHPKMTELYQSLADLNNVRFSAYRTAMKLRRLQKALCLDLLSLSAAC
DALDQHNLKQNDQPMDILQIINCLTTIYDRLEQEHNNLVNVPLCVDMCLN
WLLNVYDTGRTGRIRVLSFKTGIISLCKAHLEDKYRYLFKQVASSTGFCD
QRRLGLLLHDSIQIPRQLGEVASFGGSNIEPSVRSCFQFANNKPEIEAAL
FLDWMRLEPQSMVWLPVLHRVAAAETAKHQAKCNICKECPIIGFRYRSLK
HFNYDICQSCFFSGRVAKGHKMHYPMVEYCTPTTSGE

### D. Others

Many high-molecular weight proteins have repetitive internal domains. Dystrophin exemplifies the large class of proteins for which limited three dimensional structural information is available for the repetitive internal domains. A conceptually identical approach may be applicable to other genetic diseases. For instance, the common inherited coagulopathy von Willebrand's disease is caused by mutations in the 8 kilobase coding sequence for a protein with multiple repeats (“vWF”). Recently published studies have revealed that transgenic expression of the recombinant protein in the liver is sufficient to treat the disease, but the coding sequence is too large for a single AAV genome, and trans-splicing between two AAV genomes is too inefficient to achieve therapeutic levels of recombinant protein expression. There is no crystal structure for the entire protein, but analysis by the methods outlined herein immediately suggest opportunities to create a miniaturized nano-vWF protein that may substitute for full length vWF.

Von Willebrand disease is typically an inherited disease caused by variations (mutations) in the VWF gene. The VWF gene provides instructions for making a blood clotting protein called von Willebrand factor, which is important for forming blood clots and preventing further blood loss after an injury. If von Willebrand factor does not function normally or too little of the protein is available, blood clots cannot form properly. VWF gene mutations that reduce the amount of von Willebrand factor or cause the protein to function abnormally (or not at all) are responsible for the signs and symptoms associated with the condition. These variations may be inherited in an autosomal dominant or autosomal recessive manner, or may occur for the first time in the affected person without any other cases in the family (known as a de novo mutation). See, e.g., ghr.nlm.nih.gov/condition/von-willebrand-disease. It would be understood by one of skill in the art that, any composition, regiment, aspect, embodiment and method described herein across the Specification are intended to be applied to Von Willebrand disease von, a mutant von Willebrand factor, a nucleic acid sequence encoding a mutant von Willebrand factor, or a vector comprising such coding sequence.

Von Willebrand factor (vWF) is important in the maintenance of hemostasis. It promotes adhesion of platelets to the sites of vascular injury by forming a molecular bridge between sub-endothelial collagen matrix and platelet-surface receptor complex GPIb-IX-V. It also acts as a chaperone for coagulation factor VIII, delivering it to the site of injury, stabilizing its heterodimeric structure and protecting it from premature clearance from plasma. There are 2 isoforms of von Willebrand factor. Isoform 1 is considered as the canonical sequence and has an amino acid sequence with UniProtKB identifier: P04275-1, which is incorporated herein. The other isoform is isoform 2 with UniProtKB identifier: P04275-2, sequence of the isoform is incorporated herein. “Full-length” vWF may refers to isoform 1. In certain embodiments, “Full-length” vWF may refers to isoform 2 or other homologs of vWF. Homologs of von Willebrand factor have been identified in a variety of organisms, including mouse (UniProt Q8CIZ8), rat (UniProt Q62935), pig (UniProt Q28833), and dog (UniProt Q28295). Possible nucleic acid sequence encoding von Willebrand factor or any other isoform or homolog thereof is available publicly, See, e.g., NCBI Reference Sequences: NM_000552.4, X04385.1, M10321.1, X04146.1, AK128487.1, AK297600.1, AK292122.1, BC069030.1, BC022258.1, U81237.1, K03028.1, M17588.1, AF086470.1, and X02672.1, each of which is incorporated herein.

As used herein, “mutant von Willebrand factor” or “mutant vWF” refers to a von Willebrand factor having an internal deletion of repeat(s) and a splice junction joining two repeats, wherein the splice junction sites are within a repeat instead of between repeats. In certain embodiment, the splice junction is determined via a method similar to that of A. Splice junctions in the helices, I, Dystrophin, utrophin and others, or of any Examples. Identification or characterization of the repeat(s) in vWF may be determined by the Hidden Markov Model (HMM), or any other conventional methods. See, for example, Zhou Y F et al. Sequence and structure relationships within von Willebrand factor. Blood. 2012 Jul. 12; 120 (2): 449-58. doi: 10.1182/blood-2012-01-405134. Epub 2012 Apr. 6.; Sadler J E. Biochemistry and genetics of von Willebrand factor. Annu Rev Biochem. 1998; 67:395-424; and Perkins S J, et al. The secondary structure of the von Willebrand factor type A domain in factor B of human complement by Fourier transform infrared spectroscopy. Its occurrence in collagen types VI, VII, XII and XIV, the integrins and other proteins by averaged structure predictions. J Mol Biol. 1994 Apr. 22; 238 (1): 104-19. Nucleic acid sequence encoding mutant vWF may be generated via tools for reverse-translation. Furthermore, the coding sequences might be codon-optimized for expression in a subject, e.g., human, mice, rat or dog.

It should be understood that any composition described herein are intended to be applied to other compositions, regiments, aspects, embodiments and methods described across the Specification.

### II. Expression Cassette

Provided herein is an expression cassette which comprises a nucleic acid sequence encoding a dystrophin superfamily triple splice mutant protein under control of regulatory sequences which direct expression thereof.

As used herein, the term “expression” or “gene expression” refers to the process by which information from a gene is used in the synthesis of a functional gene product. The gene product may be a protein, a peptide, or a nucleic acid polymer (such as a RNA, a DNA or a PNA). In certain embodiments, the functional gene product is a dystrophin superfamily triple splice mutant protein. In certain embodiments, the terms “gene” “minigene” and “transgene” refers to the sequence coding a dystrophin superfamily triple splice mutant protein, for example, nano-dystrophin or nano-utrophin.

As used herein, an “expression cassette” refers to a nucleic acid polymer which comprises a coding sequences, promoter, and may include other regulatory sequences therefor, which cassette may be packaged into a vector.

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with a coding sequence and expression control sequences that act in trans or at a distance to control the coding sequence. In certain embodiment, the coding sequence encodes a dystrophin superfamily triple splice mutant protein.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

Identity or similarity with respect to a sequence is defined herein as the percentage of amino acid residues in the candidate sequence that are identical (i.e., same residue) or similar (i.e., amino acid residue from the same group based on common side-chain properties, see below) with the peptide and polypeptide regions provided herein, after aligning the sequences and introducing gaps, if necessary, to achieve the maximum percent sequence identity. Percent (%) identity is a measure of the relationship between two polynucleotides or two polypeptides, as determined by comparing their nucleotide or amino acid sequences, respectively. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. There are a number of algorithms, and computer programs based thereon, which are available to be used the literature and/or publicly or commercially available for performing alignments and percent identity. The selection of the algorithm or program is not a limitation of the present invention.

Examples of suitable alignment programs including, e.g., the software CLUSTALW under Unix and then be imported into the Bioedit program (Hall, T. A. 1999, BioEdit: a user-friendly biological sequence alignment editor and analysis program for Windows 95/98/NT. Nucl. Acids. Symp. Ser. 41:95-98); the Clustal Omega available from EMBL-EBI (Sievers, Fabian, et al. “Fast, scalable generation of high-quality protein multiple sequence alignments using Clustal Omega.” Molecular systems biology 7.1 (2011): 539 and Goujon, Mickael, et al. “A new bioinformatics analysis tools framework at EMBL-EBI.” Nucleic acids research 38.suppl 2 (2010): W695-W699); the Wisconsin Sequence Analysis Package, version 9.1 (Devereux J. et al., Nucleic Acids Res., 12:387-395, 1984, available from Genetics Computer Group, Madison, Wis., USA). The programs BESTFIT and GAP, may be used to determine the % identity between two polynucleotides and the % identity between two polypeptide sequences.

Other programs for determining identity and/or similarity between sequences include, e.g, the BLAST family of programs available from the National Center for Biotechnology Information (NCB), Bethesda, Md., USA and accessible through the home page of the NCBI on the world wide web at ncbi.nlm.nih.gov), the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used; and FASTA (Pearson W. R. and Lipman D. J., Proc. Natl. Acad. Sci. USA, 85:2444-2448, 1988, available as part of the Wisconsin Sequence Analysis Package). SeqWeb Software (a web-based interface to the GCG Wisconsin Package: Gap program).

In one embodiment, the expression cassette is designed for expression and secretion in a subject, for example, human, rat, mouse or dog. In one embodiment, the expression cassette is designed for expression in the muscle, including cardiac muscles, skeletal muscles and smooth muscles.

In certain embodiments, the regulatory control elements include a promoter sequence as part of the expression control sequences, e.g., located between the selected 5′ ITR sequence and the coding sequence. Constitutive promoters, regulatable promoters [see, e.g., WO 2011/126808 and WO 2013/04943], tissue specific promoters (see, e.g., on the world wide web at invivogen.com/tissue-specific-promoter), or a promoter responsive to physiologic cues may be used may be utilized in the vectors described herein. In certain embodiment, muscle-specific promoter may be used, e.g., a muscle creatine kinase (MCK) promoter, a Desmin promoter, a Mb promoter, or a promoter for Myosin-heavy polypeptide 2, Myosin, Troponin T type 3, Troponin C type 2, Myosin binding protein C, fast skeletal myosin light chain 2, Actinin a2, Vesicle-associated membrane protein 5, thyroid hormone receptor interactor 10, tropomyosin 3, Sarcoglycan γ, Myogenic differentiation 1, Myogenic factor 6 (herculin) or Calcium channel, voltage-dependent γ1. Another useful promoter is a synthetic SPc5-12 promoter, which allows for robust expression in skeletal and cardiac muscles. (see for example, Rasowo et al, European Scientific Journal, June 2014, edition vol. 10, No. 18 and US Patent Application Publication Nos. 20040192593 and 2017/0275649, all of which are incorporated herein by reference).

In certain embodiments, the regulatory control elements include a cardiac-specific cis-acting regulatory module (CS-CRM), which includes any of CS-CRM elements 1-8. In certain embodiments, the regulatory sequences include the CS-CRM4 element or the CS-CRM4 element in combination with the SPc5-12 promoter, such as the chimeric synthetic CS-CRM4/SPc5-12 promoter described previously (Rincon et al. Genome-wide computational analysis reveals cardiomyocyte-specific transcriptional Cis-regulatory motifs that enable efficient cardiac gene therapy. Mol Ther. 2015 January; 23 (1): 43-52), which is incorporated herein by reference).

Examples of constitutive promoters suitable for controlling expression of the therapeutic products include, but are not limited to chicken β-actin (CB) promoter, human cytomegalovirus (CMV) promoter, ubiquitin C promoter (UbC), the early and late promoters of simian virus 40 (SV40), U6 promoter, metallothionein promoters, EFla promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the β-actin promoter (Lai et al., Proc. Natl. Acad. Sci. USA 86:10006-10010 (1989), the long terminal repeats (LTR) of Moloney Leukemia Virus and other retroviruses, the thymidine kinase promoter of Herpes Simplex Virus and other constitutive promoters known to those of skill in the art. Examples of tissue- or cell-specific promoters suitable for use in the present invention include, but are not limited to, endothelin-I (ET-I) and Flt-I, which are specific for endothelial cells, FoxJ1 (that targets ciliated cells).

Inducible promoters suitable for controlling expression of the therapeutic product include promoters responsive to exogenous agents (e.g., pharmacological agents) or to physiological cues. These response elements include, but are not limited to a hypoxia response element (HRE) that binds HIF-Iα and β. a metal-ion response element such as described by Mayo et al. (1982, Cell 29:99-108); Brinster et al. (1982, Nature 296:39-42) and Searle et al. (1985, Mol. Cell. Biol. 5:1480-1489); or a heat shock response element such as described by Nouer et al. (in: Heat Shock Response, ed. Nouer, L., CRC, Boca Raton, Fla., ppI67-220, 1991).

In one embodiment, expression of the coding sequence is controlled by a regulatable promoter that provides tight control over the transcription of the coding sequence, e.g., a pharmacological agent, or transcription factors activated by a pharmacological agent or in alternative embodiments, physiological cues. Promoter systems that are non-leaky and that can be tightly controlled are preferred. Examples of regulatable promoters which are ligand-dependent transcription factor complexes that may be used in the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety. An example of a non-steroidal ecdysone agonist-regulated system is the RheoSwitch Mammalian Inducible Expression System (New England Biolabs, Ipswich, MA).

Still other promoter systems may include response elements including but not limited to a tetracycline (tet) response element (such as described by Gossen & Bujard (1992, Proc. Natl. Acad. Sci. USA 89:5547-551); or a hormone response element such as described by Lee et al. (1981, Nature 294:228-232); Hynes et al. (1981, Proc. Natl. Acad. Sci. USA 78:2038-2042); Klock et al. (1987, Nature 329:734-736); and Israel & Kaufman (1989, Nucl. Acids Res. 17:2589-2604) and other inducible promoters known in the art. Using such promoters, expression of the transgene can be controlled, for example, by the Tet-on/off system (Gossen et al., 1995, Science 268:1766-9; Gossen et al., 1992, Proc. Natl. Acad. Sci. USA., 89 (12): 5547-51); the TetR-KRAB system (Urrutia R., 2003, Genome Biol., 4 (10): 231; Deuschle U et al., 1995, Mol Cell Biol. (4): 1907-14); the mifepristone (RU486) regulatable system (Geneswitch; Wang Y et al., 1994, Proc. Natl. Acad. Sci. USA., 91 (17): 8180-4; Schillinger et al., 2005, Proc. Natl. Acad. Sci. USA. 102 (39): 13789-94); the humanized tamoxifen-dep regulatable system (Roscilli et al., 2002, Mol. Ther. 6 (5): 653-63). The gene switch may be based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, Mass.) and the systems described in U.S. Pat. Nos. 6,015,709, 6,117,680, 6,479,653, 6,187,757, and 6,649,595, U.S. Publication No. 2002/0173474, U.S. Publication No. 200910100535, U.S. Pat. Nos. 5,834,266, 7,109,317, 7,485,441, 5,830,462, 5,869,337, 5,871,753, 6,011,018, 6,043,082, 6,046,047, 6,063,625, 6,140,120, 6,165,787, 6,972,193, 6,326,166, 7,008,780, 6,133,456, 6,150,527, 6,506,379, 6,258,823, 6,693,189, 6,127,521, 6,150,137, 6,464,974, 6,509,152, 6,015,709, 6,117,680, 6,479,653, 6,187,757, 6,649,595, 6,984,635, 7,067,526, 7,196,192, 6,476,200, 6,492,106, WO 94/18347, WO 96/20951, WO 96/06097, WO 97/31898, WO 96/41865, WO 98/02441, WO 95/33052, WO 99110508, WO 99110510, WO 99/36553, WO 99/41258, WO 01114387, ARGENT™ Regulated Transcription Retrovirus Kit, Version 2.0 (9109102), and ARGENT™ Regulated Transcription Plasmid Kit, Version 2.0 (9109/02), each of which is incorporated herein by reference in its entirety. The Ariad system is designed to be induced by rapamycin and analogs thereof referred to as “rapalogs”. Examples of suitable rapamycins are provided in the documents listed above in connection with the description of the ARGENT system. In one embodiment, the molecule is rapamycin [e.g., marketed as Rapamune by Pfizer]. In another embodiment, a rapalog known as AP21967 [ARIAD] is used. Examples of these dimerizer molecules that can be used in the present invention include, but are not limited to rapamycin, FK506, FK1012 (a homodimer of FK506), rapamycin analogs (“rapalogs”) which are readily prepared by chemical modifications of the natural product to add a “bump” that reduces or eliminates affinity for endogenous FKBP and/or FRAP. Examples of rapalogs include, but are not limited to such as AP26113 (Ariad), AP1510 (Amara, J. F., et al., 1997, Proc Natl Acad Sci USA, 94 (20): 10618-23) AP22660, AP22594, AP21370, AP22594, AP23054, AP1855, AP1856, AP1701, AP1861, AP1692 and AP1889, with designed ‘bumps’ that minimize interactions with endogenous FKBP. Still other rapalogs may be selected, e.g., AP23573 [Merck].

Other suitable enhancers include those that are appropriate for a desired target tissue indication. In one embodiment, the expression cassette comprises one or more expression enhancers. In one embodiment, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g, the chicken beta-actin intron. Other suitable introns include those known in the art, e.g., such as are described in WO 2011/126808. Examples of suitable polyA sequences include, e.g., rabbit binding globulin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence [see, e.g., M A Zanta-Boussif, et al, Gene Therapy (2009) 16:605-619. In one embodiment, the enhancer is a double or triple tandem MCK enhancer.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, an SV40 polyA, or a synthetic polyA may be included in an expression cassette.

It should be understood that the compositions in the expression cassette described herein are intended to be applied to other compositions, regiments, aspects, embodiments and methods described across the Specification.

### III. Vectors

In certain embodiments, the nucleic acid sequence encoding a dystrophin superfamily triple splice mutant protein are engineered in a vector, including a viral vector and a non-viral vector.

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. Such vectors preferably have one or more origin of replication, and one or more site into which the coding sequences or an expression cassette can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a muscle cell, a cell of smooth muscle, a cell of cardiac muscle or a cell of skeletal muscle.

The term “exogenous” as used to describe a nucleic acid sequence or protein means that the nucleic acid or protein does not naturally occur in the position in which it exists in a chromosome, or host/target cell. An exogenous nucleic acid sequence also refers to a sequence derived from and inserted into the same host cell or subject, but which is present in a non-natural state, e.g. a different copy number, or under the control of different regulatory elements.

As used herein, the term “target cell” refers to any target cell in which expression of the dystrophin superfamily triple splice mutant protein is desired. In certain embodiments, the term “target cell” is intended to reference the cells of the subject being treated for MD, including DMD and BMD. Examples of target cells may include, but are not limited to, a liver cell, a kidney cell, a muscle cell, a cell of smooth muscle, a cell of cardiac muscle or a cell of skeletal muscle. In certain embodiments, the vector is delivered to a target cell ex vivo. In certain embodiments, the vector is delivered to the target cell in vivo.

A non-viral vector may be a plasmid carrying an expression cassette which includes, at a minimum, nucleic acid sequence encoding a dystrophin superfamily triple splice mutant protein and optionally, a promoter or other regulatory elements, which is delivered to the heart. Non-viral delivery of nucleic acid molecules to smooth and cardiac muscle systems may include chemical or physical methods. Chemical methods include the use of cationic liposomes (“lipoplex”), polymers (“polyplex”), combinations of the two (“lipopolyplex”), calcium phosphate, and DEAE dextran. Additionally, or optionally, such nucleic acid molecules may be used in a composition further comprising one or more reagents, including, e.g., liposomal reagents such as, e.g., DOTAP/DOPE, Lipofectin, Lipofectamine, etc, and cationic polymers such as PEI, Effectene, and dendrimers. Such reagents are effective for transfecting smooth muscle cells. In addition to the chemical methods, a number of physical methods exist that promote the direct entry of uncomplexed DNA into the cell. These methods can include microinjection of individual cells, hydroporation, electroporation, ultrasound, and biolistic delivery (i.e., the gene gun).

In certain embodiments, an expression cassette comprising the nucleic acid sequence encoding a dystrophin superfamily triple splice mutant protein is carried by a viral vector, e.g., a recombinant adenovirus, lentivirus, a bocavirus, a hybrid AAV/bocavirus (see, e.g., Yan Z et al, A novel chimeric adenoassociated virus 2/human bocavirus 1 parvovirus vector efficiently transduces human airway epithelia. Mol Ther. 2013 December; 21 (12): 2181-94. doi: 10.1038/mt.2013.92. Epub 2013 Jul. 30.), a herpes simplex virus, or adeno-associated virus. In such embodiments, the viral vector may be a replication-defective virus.

A “replication-defective virus” or “viral vector” refers to a synthetic or artificial viral particle in which a vector genome comprising an expression cassette is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

The vector may be any vector known in the art or disclosed above, including naked DNA, a plasmid, phage, transposon, cosmids, episomes, viruses, etc. Introduction into the host cell of the vector may be achieved by any means known in the art or as disclosed above, including transfection, and infection. One or more of the adenoviral genes may be stably integrated into the genome of the host cell, stably expressed as episomes, or expressed transiently. The gene products may all be expressed transiently, on an episome or stably integrated, or some of the gene products may be expressed stably while others are expressed transiently. Furthermore, the promoters for each of the adenoviral genes may be selected independently from a constitutive promoter, an inducible promoter or a native adenoviral promoter. The promoters may be regulated by a specific physiological state of the organism or cell (i.e., by the differentiation state or in replicating or quiescent cells) or by exogenously-added factors, for example.

Introduction of the molecules (as plasmids or viruses) into the host cell may also be accomplished using techniques known to the skilled artisan and as discussed throughout the specification. In preferred embodiment, standard transfection techniques are used, e.g., CaPO₄ transfection or electroporation. Assembly of the selected DNA sequences of the adenovirus (as well as the transgene and other vector elements into various intermediate plasmids, and the use of the plasmids and vectors to produce a recombinant viral particle are all achieved using conventional techniques. Such techniques include conventional cloning techniques of cDNA such as those described in texts [Sambrook et al, Molecular Cloning: A Laboratory Manual], use of overlapping oligonucleotide sequences of the adenovirus genomes, polymerase chain reaction, and any suitable method which provides the desired nucleotide sequence. Standard transfection and co-transfection techniques are employed, e.g., CaPO₄ precipitation techniques. Other conventional methods employed include homologous recombination of the viral genomes, plaquing of viruses in agar overlay, methods of measuring signal generation, and the like.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for a formulation. One of skill in the art may adjust these doses, depending the route of administration, and the therapeutic or vaccinal application for which the recombinant vector is employed. The levels of expression of the transgene can be monitored to determine the frequency of dosage administration. Yet other methods for determining the timing of frequency of administration will be readily apparent to one of skill in the art.

As used herein, a “vector genome” refers to the nucleic acid sequence packaged inside a vector.

### A. Replication-Defective Adenovirus Vector

In one embodiment, replication-defective adenoviral vectors are used. Any of a number of suitable adenoviruses may be used as a source of the adenoviral capsid sequence and/or in production. See, e.g., U.S. Pat. Nos. 9,617,561; 9,592,284; 9,133,483; 8,846,031; 8,603,459; 8,394,386; 8,105,574; 7,838,277; 7,344,872; 8,387,368; 6,365,394; 6,287,571; 6,281,010; 6,270,996; 6,261,551; 6,251,677; 6,203,975; 6,083,716; 6,019,978; 6,001,557; 5,872,154; 5,871,982; 5,856,152; 5,698,202. Still other adenoviruses are available from the American Type Culture Collection. In one embodiment, the adenoviral particles are rendered replication-defective by deletions in the E1a and/or E1b genes. Alternatively, the adenoviruses are rendered replication-defective by another means, optionally while retaining the E1a and/or E1b genes. The adenoviral vectors can also contain other mutations to the adenoviral genome, e.g., temperature-sensitive mutations or deletions in other genes. In other embodiments, it is desirable to retain an intact E1a and/or E1b region in the adenoviral vectors. Such an intact E1 region may be located in its native location in the adenoviral genome or placed in the site of a deletion in the native adenoviral genome (e.g., in the E3 region).

In the construction of useful adenovirus vectors for delivery of a gene to the human (or other mammalian) cell, a range of adenovirus nucleic acid sequences can be employed in the vectors. For example, all or a portion of the adenovirus delayed early gene E3 may be eliminated from the adenovirus sequence which forms a part of the recombinant virus. The function of E3 is believed to be irrelevant to the function and production of the recombinant virus particle. Adenovirus vectors may also be constructed having a deletion of at least the ORF6 region of the E4 gene, and more desirably because of the redundancy in the function of this region, the entire E4 region. Still another adenoviral vector contains a deletion in the delayed early gene E2a. Deletions may also be made in any of the late genes L1 through L5 of the adenovirus genome. Similarly, deletions in the intermediate genes IX and IVa₂ may be useful for some purposes. Other deletions may be made in the other structural or non-structural adenovirus genes. The above discussed deletions may be used individually, i.e., an adenovirus sequence for use as described herein may contain deletions in only a single region. Alternatively, deletions of entire genes or portions thereof effective to destroy their biological activity may be used in any combination. For example, in one exemplary vector, the adenovirus sequence may have deletions of the E1 genes and the E4 gene, or of the E1, E2a and E3 genes, or of the E1 and E3 genes, or of E1, E2a and E4 genes, with or without deletion of E3, and so on. As discussed above, such deletions may be used in combination with other mutations, such as temperature-sensitive mutations, to achieve a desired result.

An adenoviral vector lacking any essential adenoviral sequences (e.g., E1a, E1b, E2a, E2b, E4 ORF6, L1, L2, L3, L4 and L5) may be cultured in the presence of the missing adenoviral gene products which are required for viral infectivity and propagation of an adenoviral particle. These helper functions may be provided by culturing the adenoviral vector in the presence of one or more helper constructs (e.g., a plasmid or virus) or a packaging host cell. See, for example, the techniques described for preparation of a “minimal” human Ad vector in International Patent Application WO96/13597, published May 9, 1996, and incorporated herein by reference.

a. Helper Viruses

Thus, depending upon the adenovirus gene content of the viral vectors employed to carry the expression cassette, a helper adenovirus or non-replicating virus fragment may be necessary to provide sufficient adenovirus gene sequences necessary to produce an infective recombinant viral particle containing the expression cassette. Useful helper viruses contain selected adenovirus gene sequences not present in the adenovirus vector construct and/or not expressed by the packaging cell line in which the vector is transfected. In one embodiment, the helper virus is replication-defective and contains a variety of adenovirus genes in addition to the sequences described above. Such a helper virus is desirably used in combination with an E1-expressing cell line.

Helper viruses may also be formed into poly-cation conjugates as described in Wu et al, J. Biol. Chem., 264:16985-16987 (1989); K. J. Fisher and J. M. Wilson, Biochem. J., 299:49 (Apr. 1, 1994). Helper virus may optionally contain a second reporter minigene. A number of such reporter genes are known to the art. The presence of a reporter gene on the helper virus which is different from the transgene on the adenovirus vector allows both the Ad vector and the helper virus to be independently monitored. This second reporter is used to enable separation between the resulting recombinant virus and the helper virus upon purification.

b. Complementation Cell Lines

To generate recombinant adenoviruses (Ad) deleted in any of the genes described above, the function of the deleted gene region, if essential to the replication and infectivity of the virus, must be supplied to the recombinant virus by a helper virus or cell line, i.e., a complementation or packaging cell line. In many circumstances, a cell line expressing the human E1 can be used to trans-complement the Ad vector. However, in certain circumstances, it will be desirable to utilize a cell line which expresses the E1 gene products can be utilized for production of an E1-deleted adenovirus. Such cell lines have been described. See, e.g., U.S. Pat. No. 6,083,716.

If desired, one may utilize the sequences provided herein to generate a packaging cell or cell line that expresses, at a minimum, the adenovirus E1 gene under the transcriptional control of a promoter for expression in a selected parent cell line. Inducible or constitutive promoters may be employed for this purpose. Examples of such promoters are described in detail elsewhere in this specification. A parent cell is selected for the generation of a novel cell line expressing any desired adenovirus gene. Without limitation, such a parent cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Virginia 20110-2209. Other suitable parent cell lines may be obtained from other sources.

Such E1-expressing cell lines are useful in the generation of recombinant adenovirus E1 deleted vectors. Additionally, or alternatively, cell lines that express one or more adenoviral gene products, e.g., E1a, E1b, E2a, and/or E4 ORF6, can be constructed using essentially the same procedures are used in the generation of recombinant viral vectors. Such cell lines can be utilized to transcomplement adenovirus vectors deleted in the essential genes that encode those products, or to provide helper functions necessary for packaging of a helper-dependent virus (e.g., adeno-associated virus). The preparation of a host cell involves techniques such as assembly of selected DNA sequences. This assembly may be accomplished utilizing conventional techniques. Such techniques include cDNA and genomic cloning, which are well known and are described in Sambrook et al., cited above, use of overlapping oligonucleotide sequences of the adenovirus genomes, combined with polymerase chain reaction, synthetic methods, and any other suitable methods which provide the desired nucleotide sequence.

In still another alternative, the essential adenoviral gene products are provided in trans by the adenoviral vector and/or helper virus. In such an instance, a suitable host cell can be selected from any biological organism, including prokaryotic (e.g., bacterial) cells, and eukaryotic cells, including, insect cells, yeast cells and mammalian cells. Particularly desirable host cells are selected from among any mammalian species, including, without limitation, cells such as A549, WEHI, 3T3, 10T1/2, HEK 293 cells or PERC6 (both of which express functional adenoviral E1) [Fallaux, F J et al, (1998), Hum Gene Ther, 9:1909-1917], Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this invention; nor is the type of mammalian cell, i.e., fibroblast, hepatocyte, tumor cell, etc.

c. Assembly of Viral Particle and Transfection of a Cell Line

Generally, when delivering the vector comprising the minigene by transfection, the vector is delivered in an amount from about 5 μg to about 100 μg DNA, and preferably about 10 to about 50 μg DNA to about 1×10⁴ cells to about 1×10¹³ cells, and preferably about 10⁵ cells. However, the relative amounts of vector DNA to host cells may be adjusted, taking into consideration such factors as the selected vector, the delivery method and the host cells selected.

### B. Lentivirus Systems

A variety of different lentivirus systems are known in the art. See, e.g., WO2001089580 A1 for a method for obtaining stable cardiovascular transduction with a lentivirus system. See, e.g., U.S. Pat. No. 6,521,457. See, also, discussion in NB Wasala et al. The evolution of heart gene delivery vectors, J Gen Med., 2011 October; 13 (10): 557-565, which is incorporated herein by reference.

### C. Recombinant AAV

In certain embodiments, the vector genome refers to the nucleic acid sequence packaged inside a vector, e.g., an rAAV. For a rAAV, such a nucleic acid sequence may contain AAV inverted terminal repeat sequences (ITRs) and an expression cassette. In one example, a vector genome contains, at a minimum, from 5′ to 3′, an AAV 5′ ITR, a nucleic acid sequence encoding a dystrophin superfamily triple splice mutant protein, and an AAV 3′ ITR. In one example, a vector genome contains, at a minimum, from 5′ to 3′, an AAV 5′ ITR, an expression cassette, and an AAV 3′ ITR. The ITRs may be from AAV2 or from aa different source AAV other than AAV2. In other embodiments, a vector genome may contain the terminal repeats (TRs) needed for self-complementary AAV vector.

In one embodiment, provided herein is a recombinant adeno-associated virus (rAAV) having an AAV capsid and a vector genome, wherein the vector genome comprises an expression cassette as described herein, or a nucleic acid sequence encoding a dystrophin superfamily triple splice mutant protein (i.e., cDNA as used herein) under the control of regulatory sequences which direct expression thereof.

In some embodiments, the dystrophin superfamily triple splice mutant protein is designed to be expressed from a recombinant adeno-associated virus, and the vector genome also contains AAV inverted terminal repeats (ITRs). In one embodiment, the rAAV is pseudotyped, i.e., the AAV capsid is from a different source AAV than that the AAV which provides the ITRs. In one embodiment, the ITRs of AAV serotype 2 are used. However, ITRs from other suitable sources may be selected. Optionally, the AAV may be a self-complementary AAV.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, Self-complementary recombinant adeno-associated virus (scAAV) vectors promote efficient transduction independently of DNA synthesis, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Where the gene is to be expressed from an AAV, the expression cassettes described herein include an AAV 5′ inverted terminal repeat (ITR) and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and/or 3′ ITRs are used. Where a pseudotyped AAV is to be produced, the ITRs in the expression are selected from a source which differs from the AAV source of the capsid. For example, AAV2 ITRs may be selected for use with an AAV capsid having a particular efficiency for targeting muscle. In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. However, other sources of AAV ITRs may be utilized.

As used herein, “recombinant AAV viral particle” or “AAV viral particle” refers to nuclease-resistant particle (NRP) which has a capsid and packaged therein a heterologous nucleic acid molecule (vector genome) comprising an expression cassette for a dystrophin superfamily triple splice mutant protein. Such an expression cassette typically contains an AAV 5′ and/or 3′ inverted terminal repeat sequence flanking a gene sequence, in which the gene sequence is operably linked to expression control sequences. Such capsid packaged therein a vector genome may also be referred to as a “full” AAV capsid. Such a rAAV viral particle is termed “pharmacologically active” when it delivers the transgene to a host cell which is capable of expressing the desired gene product carried by the expression cassette.

In many instances, rAAV particles are referred to as “DNase resistant.” However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a transgene to a host cell and protects these packaged genomic sequences from degradation (digestion) during nuclease incubation steps designed to remove contaminating nucleic acids which may be present from the production process.

As used herein, an “AAV9 capsid” is a self-assembled AAV capsid composed of multiple AAV9 vp proteins. The AAV9 vp proteins are typically produced as alternative splice variants from a nucleic acid sequence of SEQ ID NO: 10 or a sequence at least 70%, at least 75%, at least 80%, at least 85%, at least 90%, at least 95%, at least 97%, at least 99% thereto, which encodes the vp1 amino acid sequence of SEQ ID NO: 9 (GenBank accession: AAS99264). These splice variants result in proteins of different length of SEQ ID NO: 9. In certain embodiments, “AAV9 capsid” includes an AAV having an amino acid sequence which is 99% identical to SEQ ID NO: 9 (i.e., less than about 1% variation from the referenced sequence). See, also U.S. Pat. No. 7,906,111 and WO 2005/033321. Such AAV may include, e.g., natural isolates (e.g., hu31, vp1 of which is encoded by SEQ ID NO: 11; or hu32, vp1 of which is encoded by SEQ ID NO: 12), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, 8,734,809; and WO 2016/049230A1. However, in other embodiments, other variants of AAV9, or AAV9 capsids having at least about 95% identity to the above-referenced sequences may be selected. See, e.g., US Published Patent Application No. 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

Besides AAV9, other AAV vectors may be used, for example, AAV1, AAV5, AAV6, AAV8, AAV8 triple, AAV9, Anc80, Anc81 and Anc82. See., e.g, Santiago-Ortiz et al., Gene Ther., 22 (12): 934-46 (2015); US20170051257A1; and Zinn et al., Cell Rep., 12 (6): 1056-1068 (2015).

The sequences of any of the AAV capsids can be readily generated synthetically or using a variety of molecular biology and genetic engineering techniques. Suitable production techniques are well known to those of skill in the art. See, e.g., Sambrook et al, Molecular Cloning: A Laboratory Manual, Cold Spring Harbor Press (Cold Spring Harbor, NY). Alternatively, oligonucleotides encoding peptides (e.g., CDRs) or the peptides themselves can generated synthetically, e.g., by the well-known solid phase peptide synthesis methods (Merrifield, (1962) J. Am. Chem. Soc., 85:2149; Stewart and Young, Solid Phase Peptide Synthesis (Freeman, San Francisco, 1969) pp. 27-62). These and other suitable production methods are within the knowledge of those of skill in the art and are not a limitation of the present invention.

Methods of preparing AAV-based vectors are known. See, e.g., US Published Patent Application No. 2007/0036760 (Feb. 15, 2007), which is incorporated by reference herein. The use of AAV capsids having tropism for muscle cells and/or cardiac cells are particularly well suited for the compositions and methods described herein. However, other targets may be selected. The sequences of AAV9 and methods of generating vectors based on the AAV9 capsid are described in U.S. Pat. No. 7,906,111; US2015/0315612; WO 2012/112832; and WO2017160360A3, which are incorporated herein by reference. In certain embodiments, the sequences of AAV1, AAV5, AAV6, AAV9, AAV8triple, Anc80, Anc81 and Anc82 are known and may be used to generate AAV vector. See, e.g., U.S. Pat. No. 7,186,552, WO 2017/180854, U.S. Pat. No. 7,282,199 B2, U.S. Pat. Nos. 7,790,449, and 8,318,480, which are incorporated herein by reference. The sequences of a number of such AAV are provided in the above-cited U.S. Pat. No. 7,282,199 B2, U.S. Pat. Nos. 7,790,449, 8,318,480, 7,906,111, WO/2003/042397, WO/2005/033321, WO/2006/110689, U.S. Pat. Nos. 8,927,514, 8,734,809; WO2015054653A3, WO-2016065001-A1, WO-2016172008-A1, WO-2015164786-A1, US-2010186103-A1, WO-2010138263-A2, and WO 2016/049230A1, and/or are available from GenBank. Corresponding methods have been described for AAV1, AAV8, and AAVrh10-like vectors. See, WO2017100676 A1; WO2017100674A1; and WO2017100704A1.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette composed of, at a minimum, AAV inverted terminal repeats (ITRs) and a transgene; and sufficient helper functions to permit packaging of the expression cassette into the AAV capsid protein. The host cell may be a 293 cell or a suspension 293 cell. See, e.g., Zinn, E., et al., as cited herein; Joshua C Grieger et al. Production of Recombinant Adeno-associated Virus Vectors Using Suspension HEK293 Cells and Continuous Harvest of Vector From the Culture Media for GMP FIX and FLT1 Clinical Vector. Mol Ther. 2016 February; 24 (2): 287-297. Published online 2015 Nov. 3. Prepublished online 2015 Oct. 6. doi: 10.1038/mt.2015.187; Laura Adamson-Small, et al. Sodium Chloride Enhances Recombinant Adeno-Associated Virus Production in a Serum-Free Suspension Manufacturing Platform Using the Herpes Simplex Virus System. Hum Gene Ther Methods. 2017 Feb. 1; 28 (1): 1-14. Published online 2017 Feb. 1. doi: 10.1089/hgtb.2016.151; US20160222356A1; and Chahal P S et al. Production of adeno-associated virus (AAV) serotypes by transient transfection of HEK293 cell suspension cultures for gene delivery. J Virol Methods. 2014 February; 196:163-73. doi: 10.1016/j.jviromet.2013.10.038. Epub 2013 Nov. 13.

Other methods of producing rAAV available to one of skill in the art may be utilized. Suitable methods may include without limitation, baculovirus expression system (e.g., baculovirus-infected-insect-cell system) or production via yeast. See, e.g., WO2005072364A2; WO2007084773A2; WO2007148971A8; WO2017184879A1; WO2014125101A1; U.S. Pat. No. 6,723,551B2; Bryant, L. M., et al., Lessons Learned from the Clinical Development and Market Authorization of Glybera. Hum Gene Ther Clin Dev, 2013; Robert M. Kotin, Large-scale recombinant adeno-associated virus production. Hum Mol Genet. 2011 Apr. 15; 20 (R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95 (6): 1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: S1525-0016 (17) 30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28 (1): 15-22. doi: 10.1089/hgtb.2016.164.; Li L et al. Production and characterization of novel recombinant adeno-associated virus replicative-form genomes: a eukaryotic source of DNA for gene transfer. PLOS One. 2013 Aug. 1; 8 (8): e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl: S80-93. doi: 10.1016/j.jip.2011.05.008; and Kotin R M, Large-scale recombinant adeno-associated virus production. Hum Mol Genet. 2011 Apr. 15; 20 (R1): R2-6. doi: 10.1093/hmg/ddr141. Epub 2011 Apr. 29.

To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25 (2): 115-25. doi: 10.1089/hgtb.2013.131. Epub 2014 Feb. 14.

In brief, the method for separating rAAV particles having packaged genomic sequences from genome-deficient AAV intermediates involves subjecting a suspension comprising recombinant AAV viral particles and AAV capsid intermediates to fast performance liquid chromatography, wherein the AAV viral particles and AAV intermediates are bound to a strong anion exchange resin equilibrated at a high (e.g., pH of 10.2 for AAV9), and subjected to a salt gradient while monitoring eluate for ultraviolet absorbance at about 260 and about 280. Although less optimal for rAAV9, the pH may be in the range of about 10.0 to 10.4. In this method, the AAV full capsids are collected from a fraction which is eluted when the ratio of A260/A280 reaches an inflection point. In one example, for the Affinity Chromatography step, the diafiltered product may be applied to a Capture Select Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

As used herein, the term “treatment” or “treating” refers to composition(s) and/or method(s) for the purposes of amelioration of one or more symptoms of MD, including DMD and BMD, restore of a desired function of the full-length dystrophin, or improvement of biomarker of disease. In some embodiments, the term “treatment” or “treating” is defined encompassing administering to a subject one or more compositions described herein for the purposes indicated herein. “Treatment” can thus include one or more of preventing disease, reducing the severity of the disease symptoms, retarding their progression, removing the disease symptoms, delaying progression of disease, or increasing efficacy of therapy in a given subject. As used herein, the term disease refers to MD, including DMD and BMD, or any other dystrophin-related disease.

It should be understood that the compositions in the vector described herein are intended to be applied to other compositions, regiments, aspects, embodiments and methods described across the Specification.

### IV. Methods and Kits

In other embodiments, methods for targeting muscle, including skeletal muscle, cardiac muscle, and/or smooth muscle, is desired. This may involve an intravenous injection or intramuscular injection. However, other routes of delivery may be selected.

In certain embodiments, the composition of the invention is specifically targeted (e.g., via direct injection) to the heart. In certain embodiments, the composition or specifically expressed in the heart (e.g., cardiomyocytes). Methods for preferentially targeting cardiac cells and/or for minimizing off-target non-cardiac gene transfer have been described. See, e.g., Matkar P N et al, Cardiac gene therapy: are we there yet? Gene Ther. 2016 August; 23 (8-9): 635-48. doi: 10.1038/gt.2016.43. Epub 2016 Apr. 29; Patent publications US20030148968A1, US20070054871A1, WO2000038518A1, U.S. Pat. No. 7,078,387B1, U.S. Pat. No. 6,162,796A, and WO1994011506A1.

In certain embodiments, a method such as that in U.S. Pat. No. 7,399,750, is used to increase the dwell time of the vector carrying the gene of interest in the heart by the induction of hypothermia, isolation of the heart from circulation, and near or complete cardiac arrest. Permeabilizing agents are an essential component of this method and are used during the administration of the virus to increase the uptake of the virus by the cardiac cells. This method is particularly well suited to viral vectors, where the gene expression may be highly specific to cardiac muscle and, in particularly in the case of rAAV vectors, expression may be maintained long-term, with no signs of myocardiac inflammation. Still another systems and techniques may used including, without limitation, e.g., a “bio-pacemaker”, such as that described in U.S. Pat. No. 8,642,747, US-2011-0112510.

In one embodiment, delivery is accomplished by the global myocardial perfusion method described in International publication number WO2005027995A2. In another embodiment, delivery is accomplished by the gene transfer methods described in International Patent Application No. PCT/US2004/031322, filed Sep. 24, 2004. Briefly, this method involves transferring a microutrophin of the invention to muscle cells by exsanguinating a region of the subject's microvasculature and delivering the complex to this region under high hydrostatic pressure using a configuration of perfusion cannulae and balloon as required to protect heart and lung to protein the organs during perfusion. A balloon catheter having a balloon that extends substantially the full length of the aorta or vessel that is inserted into the subject is provided for use in the systemic delivery of vector. In still another embodiment, the invention provides for delivery via a perfusion circuit and surgical method is provided for delivering a substance to a subject's heart in situ during cardiopulmonary bypass surgery. The perfusion circuit defines a path for re-circulating a solution containing a macromolecular complex through a coronary circulation circuit through a subject's heart during a surgical procedure in which the substance is prevented from being delivered to the subject's other organs.

In one aspect, provided herein is a pharmaceutical composition comprising a dystrophin superfamily triple splice mutant protein, a nucleic acid sequence encoding the dystrophin superfamily triple splice mutant protein, an expression cassette or a vector comprising such nucleic acid sequence in a formulation buffer (i.e., vehicle). In one embodiment, the formulation further comprises a surfactant, preservative, excipients, and/or buffer dissolved in the aqueous suspending liquid. In one embodiment, the buffer is PBS. Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration. Suitably, the formulation is adjusted to a physiologically acceptable pH, e.g., in the range of pH 6 to 8, or pH 6.5 to 7.5, pH 7.0 to 7.7, or pH 7.2 to 7.8.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate·7H2O), potassium chloride, calcium chloride (e.g., calcium chloride·2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts B R solution [Lukare Medical].

In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the vector compositions can be formulated in dosage units to contain an amount of vector that is in the range of about 1.0×10⁹ particles to about 1.0×10¹⁸ particles (to treat one subject) including all integers or fractional amounts within the range, and preferably 1.0×10¹² particles to 1.0×10¹⁴ particles for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ particles per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ particles per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ particles per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² particles per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ particles per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ particles per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ particles per dose including all integers or fractional amounts within the range. In another embodiment, the compositions are formulated to contain at least 1×10¹⁶, 2×10¹⁶, 3×10¹⁶, 4×10¹⁶, 5×10¹⁶, 6×10¹⁶, 7×10¹⁶, 8×10¹⁶, or 9×10¹⁶ particles per dose including all integers or fractional amounts within the range. In another embodiment, the compositions are formulated to contain at least 1×10¹⁷, 2×10¹⁷, 3×10¹⁷, 4×10¹⁷, 5×10¹⁷, 6×10¹⁷, 7×10¹⁷, 8×10¹⁷, or 9×10¹⁷ particles per dose including all integers or fractional amounts within the range. In one embodiment, for human application the dose can range from 1×10¹⁰ to about 1×10¹² particles per dose including all integers or fractional amounts within the range. In one embodiment, the subject is delivered a therapeutically effective amount of the vectors described herein. As used herein, a “therapeutically effective amount” refers to the amount of the composition comprising the nucleic acid sequence encoding a dystrophin superfamily triple splice mutant protein which delivers and expresses in the target cells an amount of enzyme sufficient to achieve efficacy. Or a “therapeutically effective amount” refers to the amount of the composition comprising the dystrophin superfamily triple splice mutant protein which delivers to a subject. In one embodiment, the dosage of the vector is about 1×10⁹ particles (e.g., genome copies, GC) per kg of body mass to about 1×10¹⁶ particles per kg of body mass, including all integers or fractional amounts within the range and the endpoints. In another embodiment, the dosage is 1×10¹⁰ particles per kg of body mass to about 1×10¹³ particles per kg of body mass.

Dosages of the vector will depend primarily on factors such as the condition being treated, the age, weight and health of the patient, and may thus vary among patients. For example, a therapeutically effective human dosage of the vector is generally in the range of from about 1 ml to about 100 ml of solution containing concentrations of from about 1×10⁷ to 1×10¹⁶ genomes or particles vector. The dosage will be adjusted to balance the therapeutic benefit against any side effects and such dosages may vary depending upon the therapeutic application for which the recombinant vector is employed. The levels of expression of the transgene can be monitored to determine the frequency of dosage resulting in vectors, preferably AAV vectors containing the minigene. Optionally, dosage regimens similar to those described for therapeutic purposes may be utilized for immunization using the compositions of the invention.

Optionally, therapy with a dystrophin superfamily triple splice mutant protein (e.g., nano-utrophin or nano-dystrophin) or a vector expressing a dystrophin superfamily triple splice mutant protein can be combined with other therapies.

Expression of the dystrophin superfamily triple splice mutant protein (e.g., nano-utrophin or nano-dystrophin) may be detected by immunofluorescent staining and immunoblotting (Western blotting). The dystrophin superfamily triple splice mutant protein (e.g., nano-utrophin or nano-dystrophin) therapy may be monitored by measuring missing DAP complexes on the myofiber plasma membrane, including the sarcoglycan complex which is typically not found in untreated dystrophic muscle due to the primary deficiency of dystrophin. Alternatively, the dystrophin superfamily triple splice mutant protein (e.g., nano-utrophin or nano-dystrophin) therapy can be monitored by assessing that muscle is protected from pathological phenotypes.

In one aspect, the invention provides a kit for use by a clinician or other personnel. Typically, such a kit will contain a mutant protein or a vector of the invention and, optionally, instructions for reconstitution and/or delivery thereof. In another embodiment, the kit will contain a mutant protein or a vector in a physiologically compatible saline solution and, optionally, instructions for dilution, and performing a method as described herein.

The kit of the invention may also contain a balloon catheter to facilitate somatic gene transfer as described (International Patent Application No. PCT/US2004/030463, or by the gene transfer methods described in International Patent Application No. PCT/US2004/031322, filed Sep. 24, 2004), oxygen-transporting agent and/or at least one disposable element of an extracorporeal circulatory support and oxygenation system. For example, at least one disposable element can be an oxygenator having a hollow body, a liquid inlet in fluid communication with the interior of the body, a liquid outlet in fluid communication with the interior of the body, a gas inlet for providing gas to the interior of a gas chamber, at least one gas-permeable membrane separating the gas chamber from the interior of the body, and a gas outlet for permitting gas to exit from the gas chamber, whereby gas exchange is enabled between a fluid in the interior of the body and a gas in the gas chamber. The oxygenator may be constructed as described in U.S. Pat. No. 6,177,403, wherein the gas-permeable membrane comprises PTFE tubing extending within at least a portion of the tube, and wherein the gas chamber comprises the interior of the PTFE tubing.

It should be understood that the compositions in the Methods and Kits described herein are intended to be applied to other compositions, regiments, aspects, embodiments and methods described across the Specification.

The term “a” or “an” refers to one or more. As such, the terms “a” (or “an”), “one or more,” and “at least one” are used interchangeably herein.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be interpreted and described using “consisting of” or “consisting essentially of” language.

The term “about” encompasses a variation within and including ±10%, unless otherwise specified.

Unless defined otherwise in this specification, technical and scientific terms used herein have the same meaning as commonly understood by one of ordinary skill in the art and by reference to published texts, which provide one skilled in the art with a general guide to many of the terms used in the present application.

### EXAMPLE

The primary structure of dystrophin and the molecular basis of the milder-than-DMD disease BMD suggested a conceptually simple means of constructing smaller, partially functional proteins for therapy in DMD. Internal deletions of single, contiguous portions of a long repetitive domain of dystrophin were used to achieve BMD-like partial length dystrophin variants which localize to the cellular address normally occupied by full-length dystrophin, and thereby partially substitute for the dystrophin's key physiological function(s). It was widely assumed that, based on the view that dystrophin serves as a molecular “shock absorber”, that the protein's full length would be required for normal function in this role. The anticipated result was that under appropriate testing this entire class of recombinant proteins would confer BMD-like phenotypes to DMD patients. No somatically delivered partial length recombinant dystrophin has completely normalized the most sensitive assays of pathology in preclinical studies, suggesting that vectors poised for clinical development by several teams will at best temporarily “Beckerize” the rate of disease progression in DMD. Both shorter and longer than wild type dystrophins can be associated with severe disease in Becker Muscular Dystrophy (BMD), indicating that dystrophin's mechanical role is not as simple as that of a length-dependent “shock absorber”. Such genotype/phenotype correlations in Duchenne and Becker Muscular Dystrophy served as starting points for the development of low molecular weight substitutes for Dp427, including potentially non-immunogenic derivatives of the dystrophin paralog utrophin. Based on novel insight into the mechanobiology of dystrophin and utrophin, we have developed utrophin or dystrophin variants that can be delivered to a patient to supersede the efficacy and safety of previously studied gene therapies in addressing DMD-specific limitations noted above.

### Example 1—Evolution of Titin but not Dystrophin Correlates with the Scalability of Locomotive Power

### A. Results and Discussions

In large animals, rapid locomotion is invariably powered by sarcomeric myosin, whereas the fastest moving unicellular eukaryotes and earliest branching animal lineages use ciliary dynein as the dominant locomotive power source (Colin, S. P., et al. Stealth predation and the predatory success of the invasive ctenophore Mnemiopsis leidyi. Proc Natl Acad Sci USA 107, 17223-17227 (2010); Srivastava, M. et al. The Trichoplax genome and the nature of placozoans. Nature 454, 955-960 (2008); Srivastava, M. et al. The Amphimedon queenslandica genome and the evolution of animal complexity. Nature 466, 720-726 (2010); Ryan, J. F. et al. The genome of the ctenophore Mnemiopsis leidyi and its implications for cell type evolution. Science 342, 1242592, doi: 10.1126/science.1242592 (2013); and Moroz, L. L. et al. The ctenophore genome and the evolutionary origins of neural systems. Nature 510, 109-114, doi: 10.1038/nature 13400 (2014)). Selective pressures driving the evolutionary transition from dynein to myosin must reflect geometric constraints imposed by the organelles in which these motors achieve maximal power density, with sarcomeres but not cilia amenable to three-dimensional scaling. The molecular basis of this pivotal transition is poorly understood. Here we show that the emergence of sarcomeres correlates with the phylogenomically reconstructed appearance of massive, poly-IgG-repeat containing proteins orthologous to chordate titins, whereas dystrophin and its associated complex of membrane-bound glycoproteins arose piecemeal, before the divergence of earlier branching lineages. We have identified invertebrate species that retain the inferred ancestral titin supergene structure, providing a unified view of gene rearrangements that previously obscured gene orthology and the common origin of sarcomeres in animals with radial and bilateral symmetry. Surprisingly, gene structures provide compelling evidence that the extraordinary size of dystrophin's rod domain reflects the historical legacy of a paralogous class of microtubule-binding proteins in which selection for increasing length occurred before the dawn of sarcomeres. These findings have critical implications for the mechanobiology of dystrophin and the design of miniaturized proteins for therapeutic use in muscular dystrophy (Examples 2 and 3). Our reconstruction suggests that geometric constraints on cell morphology and body plan required a strong yet pliable connection between cortical cytoskeleton and extracellular matrix before myosin could be safely arrayed into sarcomeres at the density required to power rapid, scale-independent locomotion.

Titin is the largest protein in the human proteome, serving in monomeric form as the primary scaffold for sarcomere formation (Zoghbi, M. E., Woodhead, J. L., Moss, R. L. & Craig, R. Three-dimensional structure of vertebrate cardiac muscle myosin filaments. Proc Natl Acad Sci USA 105, 2386-2390, doi: 10.1073/pnas.0708912105 (2008); and Kontrogianni-Konstantopoulos, A., Ackermann, M. A., Bowman, A. L., Yap, S. V. & Bloch, R. J. Muscle giants: molecular scaffolds in sarcomerogenesis. Physiol Rev 89, 1217-1267, doi: 10.1152/physrev.00017.2009 (2009)). In vertebrates, titin is primarily composed of immunoglobulin (IgG) and fibronectin type-III (Fn3) domains organized into “super-repeats” that form a polarized filament spanning hemi-sarcomeres, with unique N- and C-termini located within the Z-disc and M-line, respectively. However, titin-like proteins previously identified in invertebrate species are widely divergent in number, primary structure, domain composition, and length, complicating the delineation of functional orthology (Tskhovrebova, L. & Trinick, J. Titin: properties and family relationships. Nat Rev Mol Cell Biol 4, 679-689 (2003)). Our findings indicate that an “ancestral titin supergene” of this general structure has undergone extensive lineage-specific genomic re-arrangements and modular repeat expansions.

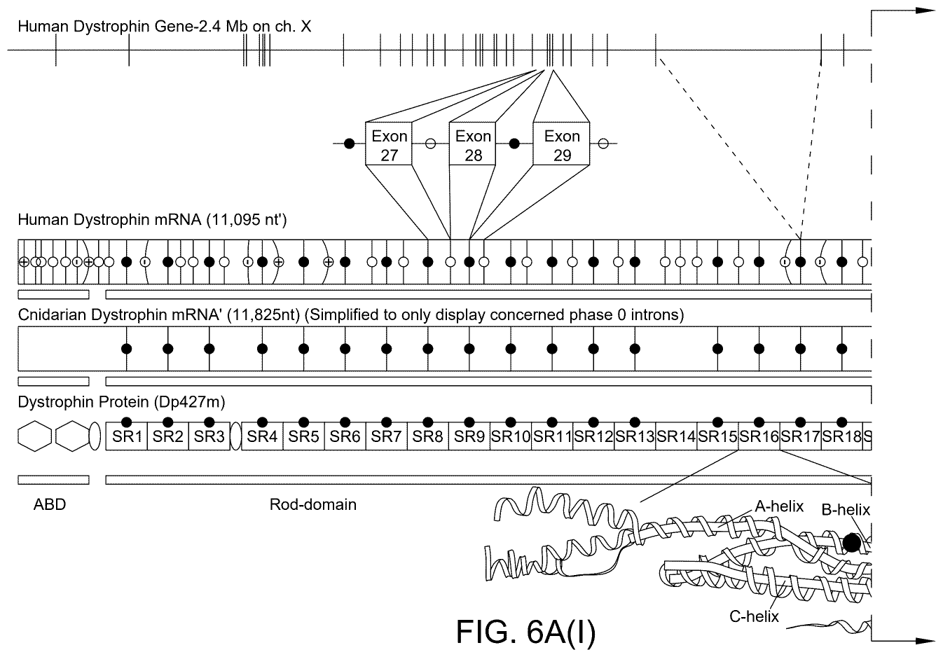

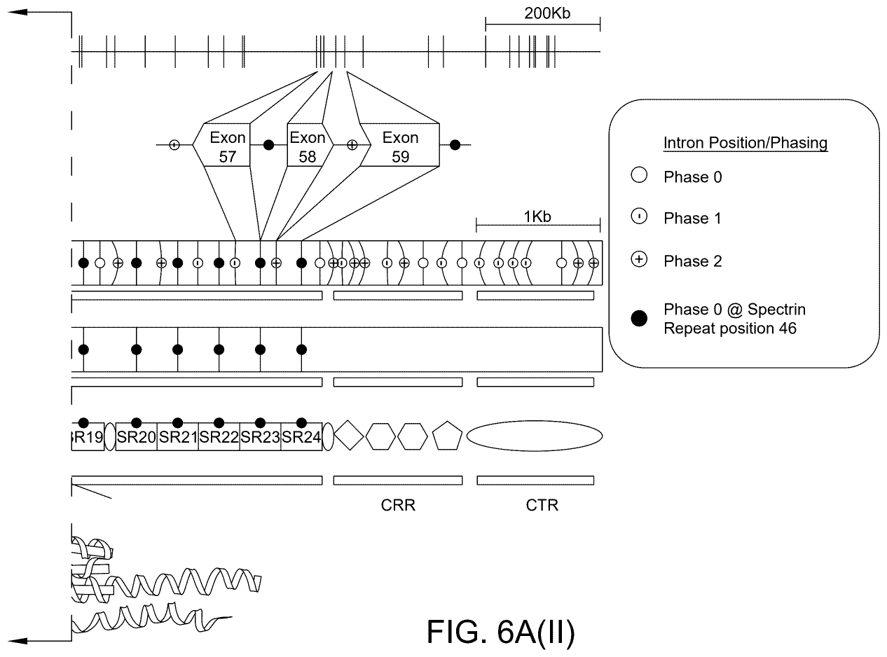

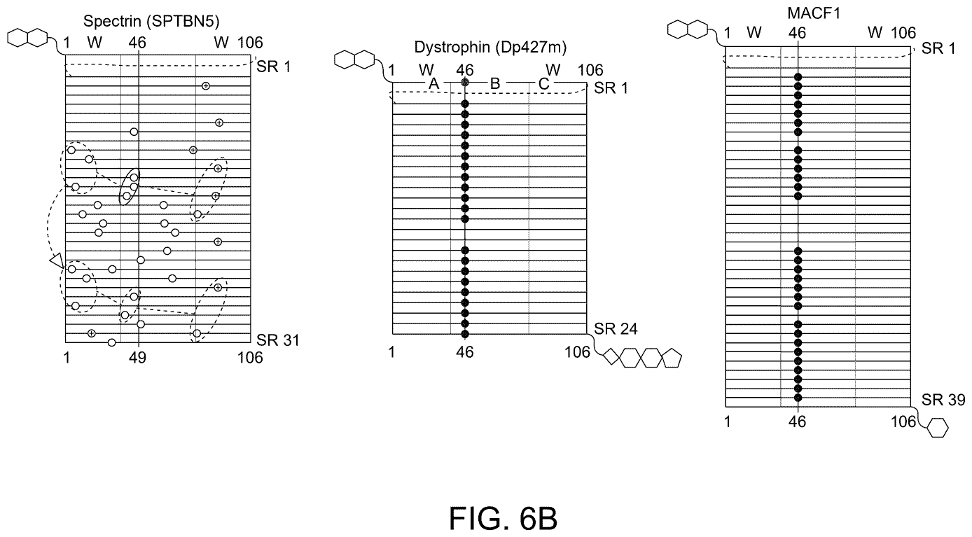

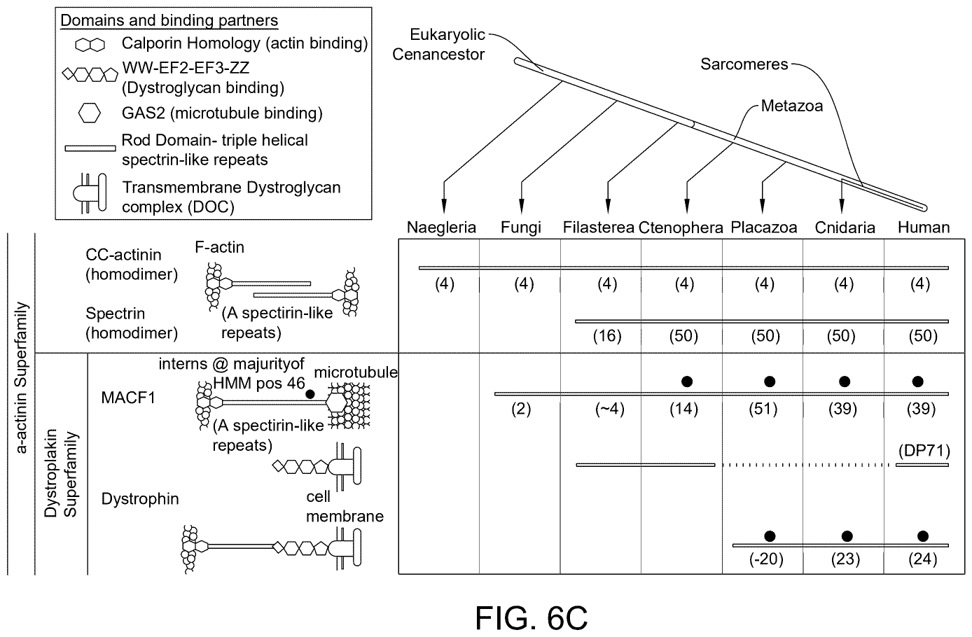

In vertebrates, the viability of striated muscle fibers under workload is dependent on membrane protection conferred by a dystrophin-dependent mechanical linkage between the outermost sarcomeres and the extracellular matrix (Hoffman, E. P., Brown, R. H., Jr. & Kunkel, L. M. Dystrophin: the protein product of the Duchenne muscular dystrophy locus. Cell 51, 919-928, doi: 0092-8674 (87) 90579-4 [pii] (1987)). Approximately 75% of dystrophin's molecular weight is contributed by a large central “rod-domain” composed of 24 spectrin repeat domains, with the flanking domains establishing adhesive contacts at opposite ends (FIG. 6A; and data not shown). Patients with Becker muscular dystrophy (BMD) can have either truncating deletions or lengthening duplications confined to exons encoding the rod domain, begging the question of whether the physiological function of dystrophin has been optimized at 24 repeats. We asked whether the number of spectrin-like repeats in dystrophin orthologs grew under selective pressure throughout metazoan phylogeny, perhaps correlating with increasing power output in selected taxa during the evolution of hierarchical predatory food chains. As shown (FIG. 6C), the ancestral dystrophin in existence before the Cnidarian-Bilatarian split is predicted to have had a rod domain of length identical to that in humans, but we could find no evidence for significantly shorter rods in earlier orthologs. Interestingly, our phylogenetic analysis provided strong evidence that the membrane-spanning dystrophin-associated protein complex emerged far earlier than metazoan multicellularity, with orthologs of nearly all disease-implicated components present in the unicellular sister groups to metazoa (data not shown). The earliest ancestral dystrophin orthologs lacked both the N-terminal actin-binding domain (ABD) and the entire rod domain and only consisted of a putative dystroglycan-binding C-terminal “WW-EF-ZZ” domain (FIG. 6A, FIG. 6E; and data not shown). The earliest branching lineage with a “modern” dystrophin ortholog (i.e. an N-terminal ABD and an elongated rod-domain) is the Placozoan species T. adherens in which the rod domain is of similar size to that of humans (data not shown). Thus dystrophin was at “full length” prior to the IgG expansions of titin and the emergence of sarcomeres; however, the evolutionary lineage of the rod domain is hitherto unresolved because significant sequence divergence among homologous proteins invites the “long branch attraction” artifact.

We addressed this problem by identifying ancestral character states that evolve more slowly than individual amino acids or nucleotides within a sequence: the position and phase of introns relative to hidden Markov models for encoded protein domains. We discovered that spectrin repeats of dystrophin and MACF1 share conserved phase 0 introns in a visually striking pattern at HMM consensus position 46, sharply contrasting with the randomly distributed introns of the spectrin genes (FIG. 6A and FIG. 6B). Illustration of the evolutionary stasis of the relevant intron positions is accentuated by our depiction of only those shared in orthologous genes of remotely related species (e.g. note evidence of the ancestral partial gene duplication that extended the beta heavy spectrin ORF by 13 repeats) (FIG. 6B). This rod-domain character state analysis identifies an MACF ortholog, not a beta-spectrin, as the proximate donor of the dystrophin CH and rod domains, suggesting the name “dystroplakin” for this cladistic group (FIG. 6C). The gene structures constitute strong evidence that dystrophin arose evolutionarily when a partial duplication of a gene encoding the N-terminal portion of an ancestral MACF1-like spectroplakin became linked in cis to the gene encoding the ancestral Dp71-like (WW-EF-ZZ) dystrophin ortholog. In the giant MACF orthologs of selected lineages there is evidence for recent tandem duplication of exons encoding spectrin repeats. This supports a reconstruction in which the selective pressure for progressive lengthening of the rod domain occurred (and in some lineages continues) in the cellular context of a microtubule-actin crosslinking “strut”, but not in that of dystrophin per se.

Comparing the molecular evolution of the repetitive domains in titin and dystrophin revealed important contrasts. Dot matrices reveal evidence for lineage-specific “treadmilling” of titin IgG and Fn3 repeat regions (data not shown), presumably on the basis of regional tandem multiplication interchangeable as long as the overall protein is long enough to facilitate sarcomerogenesis. Among dystrophin orthologs, analogous turnover of individual spectrin repeats appears to have been almost nonexistent, suggesting strong negative selection against this for at least 600 million years (data not shown). Based on these results we propose a model for the non-interchangeability of dystrophin's spectrin repeats, reflecting the protein's role in longitudinal force transmission, whereby amino acid interactions between adjacent spectrin repeats must be preserved by evolutionary coupling (Hopf, T. A. et al. Mutation effects predicted from sequence co-variation. Nat Biotechnol 35, 128-135, doi: 10.1038/nbt.3769 (2017)) to maintain tensile strength (data not shown). In this model, purifying selection has countered the novel juxtaposition, by internal gene deletion or duplication, of divergent spectrin-like repeats which had previously undergone coupled amino-acid evolution with their ancestrally adjacent partners (as observable in BMD pathogenesis). This reconstruction of ancestral events is further supported by contrasting alternative homology models of adjacent triple helices of the dystrophin rod domain on the basis the divergent templates provided by spectrins and plakins. (data not shown). In other words, the molecular evolution of dystrophin is consistent with the proposal that the rod domain's tensile strength is more important than its length, where the latter is a byproduct of its historical legacy. The metabolic cost of perpetuating a structurally redundant ancestral solution to the problem of transmembrane force transmission is inconsequentially small because of the protein's localization to an ultrathin rim of cytoskeletal cortex. This concept has critical implications for the design of transgenes for disease therapeutics, as demonstrated in detail in Examples 2 and 3.

### B. Materials and Methods

RNA-Seq: The reference transcriptome for Nematostella vectensis, was assembled from the original clustered ESTs published by the JGI genome assembly (Putnam, N. H. et al. Sea anemone genome reveals ancestral eumetazoan gene repertoire and genomic organization. Science 317, 86-94 (2007)) along with transcriptomes produced from a clonal lineage originating in New Jersey, USA, (strain NJ3) generated by the Finnerty Lab [Lubinski, et al., in revision]. Redundant contigs were removed from the merged assembly using CD-HIT with a cutoff of 100% sequence identity.

Basic local alignment search tool (BLAST) searches: Genomes were blasted using BLASTp and/or tBLASTn algorithms with pre-set parameters (BLOSUM62 matrix, expected E value threshold: 10, gap cost existence: 11, gap cost extension: 1). In cases where full-length homologs were not identified, the genomic region surrounding the highest scoring partial-length hits was downloaded and de novo gene modeling was performed (see gene modeling methods section). Transcriptomes were blasted from the NCBI BLAST server using the tBLASTn algorithm against the transcriptome shotgun assembly (TSA) database, again with pre-set parameters.

Gene Modeling: In the absence of corresponding RNAseq data, protein-coding gene models were derived from programs of the FGENESH suite (on the world wide web at softberry.com) using organism-specific gene-finding parameters for the listed organism most closely related to the species under consideration.

Protein domain analysis: Protein domains were analyzed by running the primary amino acid sequence against the Pfam, TIGRFAM, CATH-Gene3D, Superfamily, and PIRSF protein family HMM databases using the European Bioinformatics Institute's HMMscan function of the HMMER software package (on the world wide web at ebi.ac.uk/Tools/hmmer/-search/hmmscan) (Finn, R. D. et al. HMMER web server: 2015 update. Nucleic Acids Res 43, W30-38, doi: 10.1093/nar/gkv397 (2015)).

Spectrin Repeat Alignment for Intron Position/Phase Identification: All Pfam profile-HMM identifiable spectrin repeat domains were aligned to the Pfam spectrin repeat consensus sequence within HMMscan. These spectrin repeats were sequentially aligned into a multiple sequence alignment according to their alignment relative to the consensus sequence.

Intron Position/Phase Identification: ORF annotated cDNA sequences were aligned to their encoding genomic scaffold using the dot matrix function in Mac Vector (v15.1) (macvector.com) with 94% sequence identity cut-off. The position and phase of the introns were identified as breakpoints in the alignment. Each intron position and phase was confirmed by the presence of consensus splice site donor (-GT) and acceptor sites (AG-) present within the genomic DNA sequence immediately after and before each 100% identity aligned block, respectively.

Inferred Ancestral Intron Identification: Inferred ancestral introns are those that are shared between orthologous proteins in H. sapiens and either A. queenslandica or N. vectensis.

Dot matrices: cDNA/DNA, protein/genomic DNA, and protein/protein dot matrices were generated within MacVector (v15.1) (macvector.com).

Homology modeling of dystrophin spectrin repeats: Phyre2 was used to model adjacent spectrin repeats from human dystrophin (on the world wide web at sbg.bio.ic.ac.uk/phyre2/html/page.cgi?id=index) (Kelley, L. A., Mezulis, S., Yates, C. M., Wass, M. N. & Sternberg, M. J. The Phyre2 web portal for protein modeling, prediction and analysis. Nat Protoc 10, 845-858, doi: 10.1038/nprot.2015.053 (2015)). Distinct homologymodels were generated using the crystal structure of either beta2-spectrin (PDB-ID=3EDV) (Davis, L. et al. Localization and structure of the ankyrin-binding site on beta2-spectrin. J Biol Chem 284, 6982-6987, doi: 10.1074/jbc.M809245200 (2009)) or plectin (PDBID=5J1G) (Ortega, E. et al. The Structure of the Plakin Domain of Plectin Reveals an Extended Rod-like Shape. J Biol Chem 291, 18643-18662, doi: 10.1074/jbc.M116.732909 (2016)) as the template for the homology model.

Data Availability: Sequence data used to support the findings in this paper are provided in the Supplementary Information. All other data are available from the corresponding author upon request.

### Example 2—Effective Gene Therapy for Muscular Dystrophy Using AAV-Mediated Delivery of Micro-Utrophin

### A. Results and Discussion

The essential protein product of the Duchenne muscular dystrophy (DMD) gene is dystrophin (Hoffman, E. P., Brown, R. H., Jr. & Kunkel, L. M. Dystrophin: the protein product of the Duchenne muscular dystrophy locus. Cell 51, 919-928, doi: 0092-8674 (87) 90579-4 [pii] (1987)), a rod-like 427 kd protein (Koenig, M., Monaco, A. P. & Kunkel, L. M. The complete sequence of dystrophin predicts a rod-shaped cytoskeletal protein. Cell 53, 219-226 (1988)) that protects striated myocytes from contraction-induced injury (Petrof, B. J., Shrager, J. B., Stedman, H. H., Kelly, A. M. & Sweeney, H. L. Dystrophin protects the sarcolemma from stresses developed during muscle contraction. Proceedings of the National Academy of Sciences of the United States of America 90, 3710-3714 (1993)) by linking the cortical cytoskeleton to the extracellular matrix (Ibraghimov-Beskrovnaya, O. et al. Primary structure of dystrophin-associated glycoproteins linking dystrophin to the extracellular matrix. Nature 355, 696-702, doi: 10.1038/355696a0 (1992)). Most patients with DMD have multi-exon frame-shifting deletions, while many with the milder allelic disease Becker M D have frame-preserving mutations that change the length of dystrophin's 150 nm rod domain (Monaco, A. P., Bertelson, C. J., Liechti-Gallati, S., Moser, H. & Kunkel, L. M. An explanation for the phenotypic differences between patients bearing partial deletions of the DMD locus. Genomics 2, 90-95 (1988); and Koenig, M. et al. The molecular basis for Duchenne versus Becker muscular dystrophy: correlation of severity with type of deletion. American journal of human genetics 45, 498-506 (1989)). Our analysis of the deep evolutionary history of dystrophin suggests that the rod domain was coopted from a longer cytoskeletal protein, and arose prior to the emergence of powerful striated muscle (Example 1). Here we show that a codon-optimized synthetic transgene encoding a non-immunogenic, 25 nm substitute for dystrophin, rationally designed from the paralogous protein utrophin (Tinsley, J. M. et al. Primary structure of dystrophin-related protein. Nature 360, 591-593, doi: 10.1038/360591a0 (1992)) to preserve the tensile strength of the miniaturized rod domain, prevents the most deleterious histological and physiological aspects of muscular dystrophy in animal models. Following systemic administration of an AAV vector to neonatal dystrophin-deficient mdx mice, all histological and biochemical markers of myonecrosis and regeneration are completely suppressed throughout growth to adult weight. In dystrophin-deficient dogs similarly treated at up to 4 kg body weight, systemic distribution and expression of the transgene prevented myonecrosis without cell mediated immune recognition of the protein product, suggesting protection by central immunological tolerance to full-length utrophin. These findings support a model in which tensile strength is the essential feature of the dystrophin and utrophin rods, with their 150 nm length in most lineages preserved by purifying selection against mutations that reduce length at the expense of strength.

Although internally deleted vectors derived from human adenoviruses have been used to achieve somatic transfer of 12 kb cDNAs encoding full length dystrophin, this approach has been abandoned because of the immunogenicity and limited biodistribution of the complex vector capsid (Clemens, P. R. et al. In vivo muscle gene transfer of full-length dystrophin with an adenoviral vector that lacks all viral genes. Gene therapy 3, 965-972 (1996)). Multiple vectors derived from human adeno-associated viruses (AAVs) have been shown to facilitate systemic gene transfer (Wang, B., et al. Adeno-associated virus vector carrying human minidystrophin genes effectively ameliorates muscular dystrophy in mdx mouse model. Proc Natl Acad Sci USA 97, 13714-13719. (2000); Harper, S. Q. et al. Modular flexibility of dystrophin: implications for gene therapy of Duchenne muscular dystrophy. Nat Med 8, 253-261. (2002); Gregorevic, P. et al. Systemic delivery of genes to striated muscles using adeno-associated viral vectors. Nat Med 10, 828-834 (2004); and Gregorevic, P. et al. rAAV6-microdystrophin preserves muscle function and extends lifespan in severely dystrophic mice. Nat Med 12, 787-789 (2006)), but their cloning capacity is limited to that of the wild type virus, approximately 5 kb. An equally important second constraint on gene therapy for DMD is the deletional nature of the protein deficiency in most patients, with the potential for recombinant dystrophin as a “non-self” protein (Mendell, J. R. et al. Dystrophin immunity in Duchenne's muscular dystrophy. N Engl J Med 363, 1429-1437 (2010)) to trigger chronic autoimmune myositis. We hypothesized that detailed analysis of the molecular evolution of dystrophin might inform a synthetic biology approach to both of these constraints by revealing previously un-appreciated aspects of the protein's historical legacy. Our reconstruction of the remote history of dystrophin suggested that at the protein's inception, its rod domain contained 24 repeats of the “spectrin-like” triple helical domain coopted from an N-terminal portion of another much larger strut-like cytoskeletal protein (Example 1). Crystal structures of triple helical repeats from dystrophin, utrophin, and a closely related spectroplakin suggest that amino-acid side-chain interactions between adjacent repeats create an interlocking interface critical to the strength of the rod. This principle may explain the phenotypes resulting from in-frame deletions and duplications in BMD patients and the rarity of deletions in chordate paralogs (e.g. Lamprey) as most disruptions of the native sequence of triple helical repeats have the potential to focally weaken the rod domain. To minimize the risk of creating a “weakest link”, we focused on deletions flanked on one side by the disordered domain classically labeled as “Hinge 2”, and also deleted C-terminal sequences beyond the approximate end of the ZZ domain (Ishikawa-Sakurai, M., Yoshida, M., Imamura, M., Davies, K. E. & Ozawa, E. ZZ domain is essentially required for the physiological binding of dystrophin and utrophin to beta-dystroglycan. Hum Mol Genet 13, 693-702, (2004); Hnia, K. et al. ZZ domain of dystrophin and utrophin: topology and mapping of a beta-dystroglycan interaction site. Biochem J 401, 667-677, (2007)). To take advantage of central immunological tolerance achieved through early developmental expression in the thymus (Mesnard-Rouiller, L., et al. Thymic myoid cells express high levels of muscle genes. J Neuroimmunol 148, 97-105, 2003), we mapped these deletions in dystrophin onto the paralogous protein utrophin, which diverged from dystrophin early in vertebrate evolution. Based on these considerations we synthesized transgenes based on the wild-type utrophin mRNA sequences, and then improved expression using an engineered version of the sequence. Here we report on the results obtained in blinded pre-clinical studies using vectors based on AAV9 and the derived ancestral capsid “Anc80” to systemically deliver a 3.5 kb synthetic transgene (AAV9-μU, AAV9-μUtrophin) to all striated muscles.

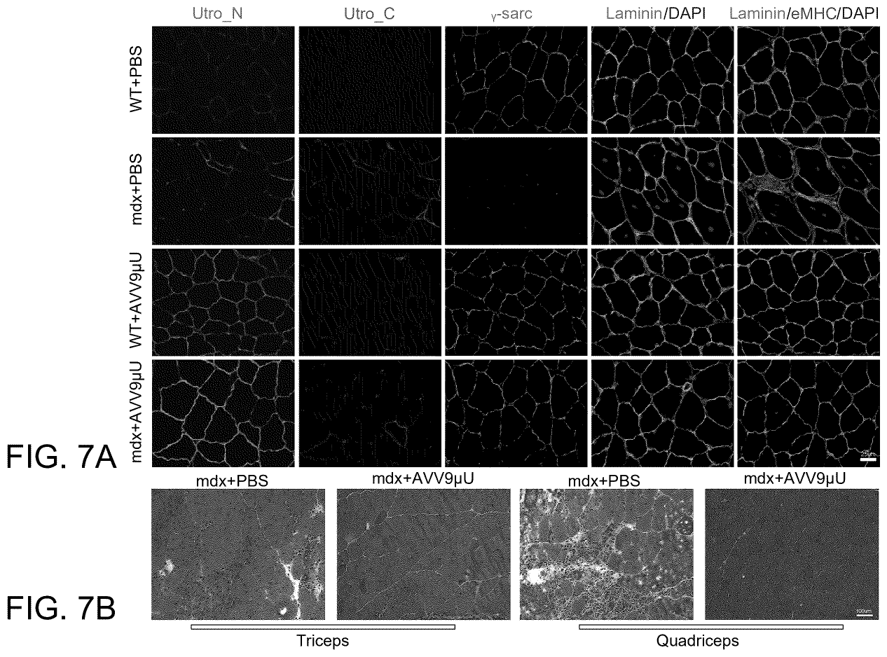

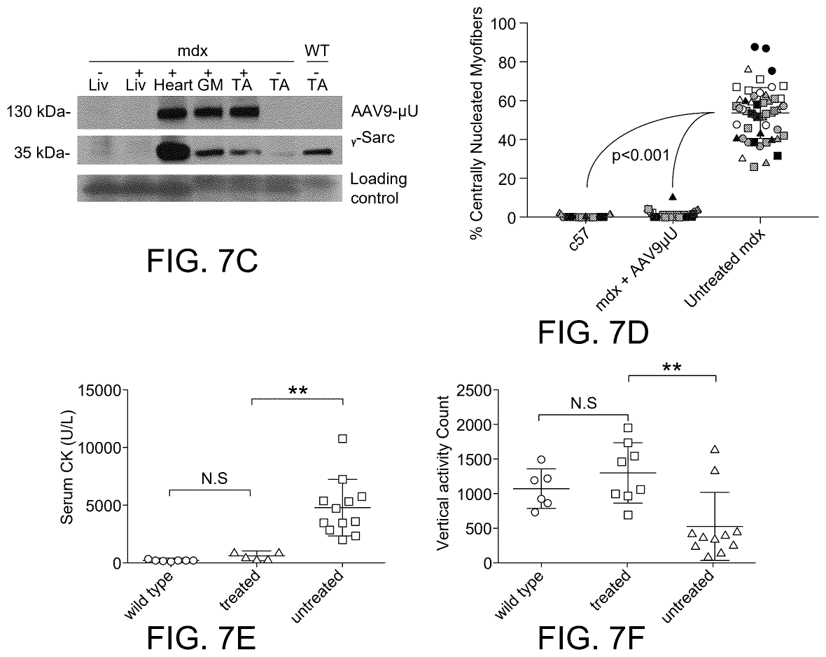

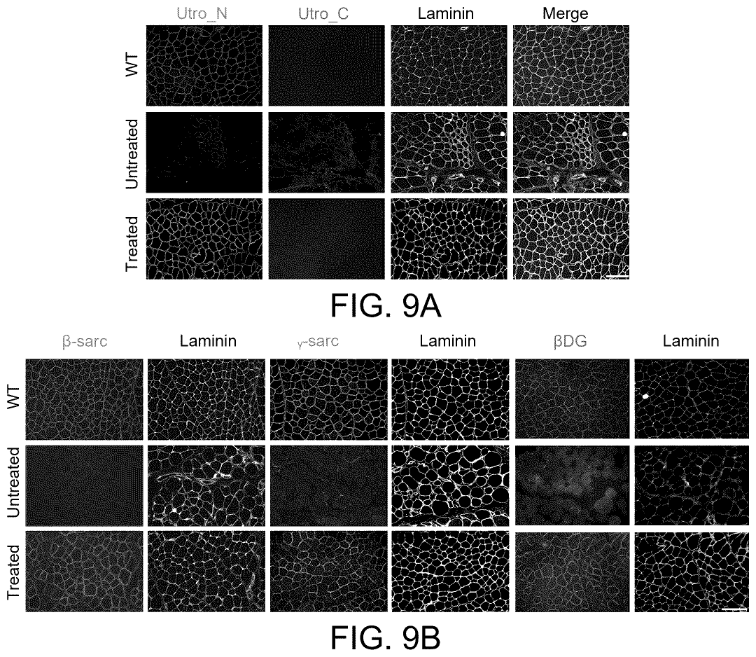

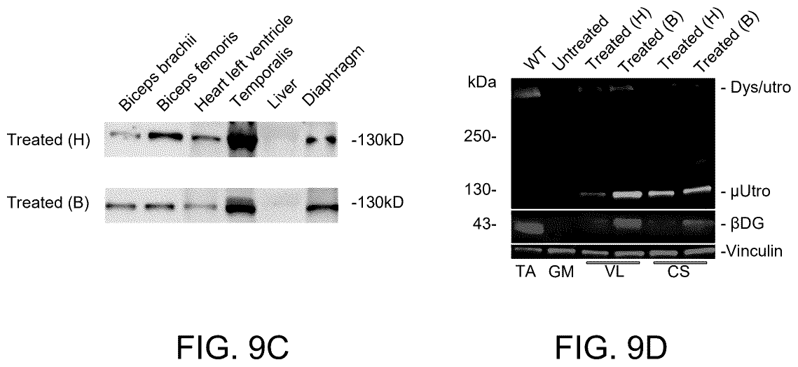

We initially performed intraperitoneal injections of doses up to 2.5×10¹² vg of AAV-μUtrophin into neonatal mdx mice weighing about 5 gm, and investigated the degree of myoprotection throughout muscle development. In these randomized, blinded studies, we observed equivalent global biodistribution to muscle with both AAV9 and Anc80, and both were well tolerated in mice without any signs of toxicity (FIG. 7A-FIG. 7C). At the 2.5×10¹² vg/mouse dose, recombinant μUtrophin was expressed at a level sufficient for qualitatively complete suppression of all tested histological signs of muscular dystrophy, including myofiber centronucleation (FIG. 7A and FIG. 7D), embryonic myosin heavy chain expression (FIG. 7A and FIG. 7B), native utrophin upregulation (FIG. 7A and FIG. 9A), MURF1 expression as a marker of protein degradation, myonuclear apoptosis (data not shown), ongoing myonecrosis, and mononuclear cell infiltration (FIG. 7B). Of these signs, centronucleation is quantitatively the most sensitive indicator of myoprotection in mdx mice since it reflects previous cycles of regeneration. For the first time, we demonstrate normalization (or prevention) of centronucleation to a level biologically indistinguishable from wild type (FIG. 7D). This observed myoprotection was associated with sustained normalization of the dystrophin-associated glycoprotein complex (DGC) in the sarcolemma of cardiac and skeletal muscles (FIG. 7A). Western blot analysis further confirmed that expressed μUtrophin protein was sufficient to stabilize the DGC (FIG. 7C). Sustained expression of μUtrophin in skeletal and cardiac muscles was observed throughout a 4-month period post vector delivery (the end point of study), indicating the durable level of myoprotection conferred by the single-dose treatment. Strikingly, creatine kinase, a biomarker that reflects sarcolemmal permeability, was statistically indistinguishable from that of wild type mice (FIG. 7E), suggesting that codon optimization and recombinant protein over-expression early in development improved the response relative to administration of alternative transgenes via tail vein injection after the onset of myopathology (Gregorevic, P. et al. Systemic delivery of genes to striated muscles using adeno-associated viral vectors. Nat Med 10, 828-834, 2004; Odom, G. L., e al. Microutrophin delivery through rAAV6 increases lifespan and improves muscle function in dystrophic dystrophin/utrophin-deficient mice. Mol Ther 16, 1539-1545, 2008; Kennedy, T. L. et al. Micro-utrophin Improves Cardiac and Skeletal Muscle Function of Severely Affected D2/mdx Mice. Mol Ther Methods Clin Dev 11, 92-105, 2018).

To test whether AAV9-μUtrophin confers functional improvement in mdx mice, we utilized an established hybrid assay which links force grip testing with a volitional component in which we non-invasively measure animals' post-force grip vertical activity (Song, Y. et al. Suite of clinically relevant functional assays to address therapeutic efficacy and disease mechanism in the dystrophic mdx mouse. J Appl Physiol 122, 593-602, 2017). Our previous studies show that this hybrid test provides one of the most sensitive, clinically relevant parameters distinguishing mdx and wild-type mice, capturing behavior causally linked to the exaggerated fatigue response of dystrophin-deficient muscle (Kobayashi, Y. M. et al. Sarcolemma-localized nNOS is required to maintain activity after mild exercise. Nature 456, 511-515, 2008). This test demonstrated an objective, dramatic, and statistically significant difference between untreated and AAV9-μUtrophin treated mdx mice, while the latter group was indistinguishable from wild type mice (FIG. 7F). Also, treated mdx mice showed not only increased voluntary wheel (8 weeks) and downhill treadmill running (16 weeks) distances relative to the untreated mice, but their ex vivo isolated EDL muscles displayed enhanced resistance to eccentric contraction-induced injury as well as enhanced muscle performance in vivo by force grip testing. These findings suggest that early over-expression of μUtrophin is capable of full phenotypic amelioration in the absence of full-length dystrophin, despite the comparatively short length of the reverse-engineered protein's rod-like linkage to the actin cytoskeleton and the lack of an R16-17 nNOS-binding motif.

These results raised the hope of achieving full, rather than BMD-like partial reversal of the pathophysiology of DMD through systemic muscle transduction; however, it was not clear whether scale-dependent differences between small and large dystrophic animals would reveal limitations to this approach. The histological and immunological consequences of μUtrophin gene transfer were further investigated in a blinded study in which five Golden Retriever Muscular Dystrophy (GRMD) dogs 4-7 days of age were randomized to intravenous administration of AAV9-μUtrophin at doses of 1×10¹³ and 3.2×10¹³ vg/kg at time of injection, without immunosuppression. Six weeks post-injection we observed robust μUtrophin expression and stabilization of wild type levels of sarcoglycan expression in the sarcolemma (data not shown). In addition, these treated dogs achieved a fourfold increase in weight similar to that of carrier females, in contrast to the previously reported weight decrement associated with immune myositis following systemic administration of xenogenic human dystrophin in the same GRMD model Kornegay, J. N. et al. Widespread muscle expression of an AAV9 human mini-dystrophin vector after intravenous injection in neonatal dystrophin-deficient dogs. Mol Ther 18, 1501-1508, 2010). This sustained μUtrophin expression was associated with visibly reduced levels of myonecrosis, mononuclear infiltration normalization of myofiber minimal Feret diameter (data not shown). At 5 and 8 weeks post-vector administration, canine interferon-γ ELISpot assays revealed no cell-mediated immunity against either the AAV capsid or the μUtrophin transgene product in our non-immunosuppressed treated GRMD dogs (data not shown). The major limitation of this proof-of-concept study stems from the 1000-fold difference between the adult weight of mdx mice and GRMD dogs, 25 g and 25 kg respectively, limiting our achievable AAV9 dose in the dog to 2.0×10¹² vg/kg based on anticipated adult weight. At this dose, the dogs would inevitably “outgrow” the vector, as did mdx mice treated with 2.15×10¹¹ vg as 5 gm neonates (data not shown). We therefore focused on relatively early histological analysis to detect recombinant μUtrophin expression, myocyte protection and the immune response to systemic vector administration.

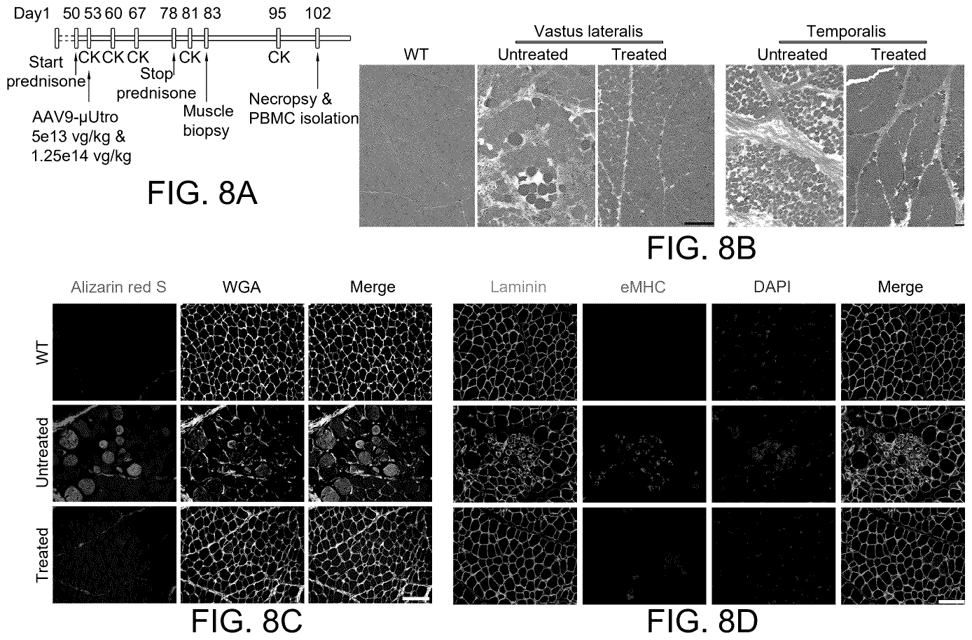

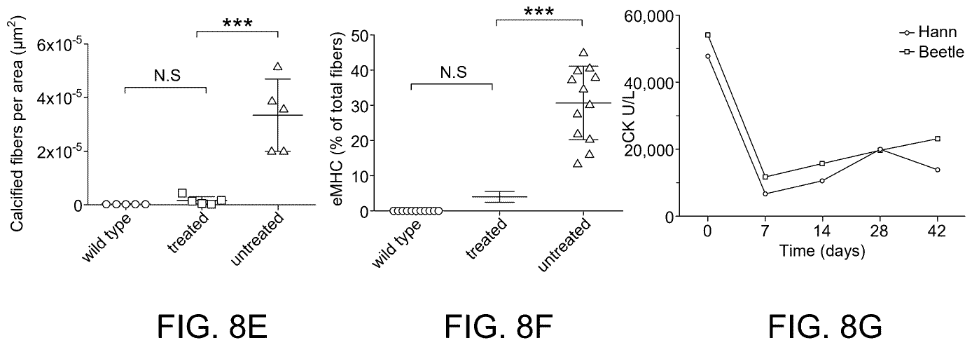

Our neonatal approach both offers the possibility of early preventative treatment before the onset of irreversible muscle damage and minimizes the risk of immune reaction against vector capsid antigens, as memory T cells recognizing the wild type AAV develop through serial environmental exposure Nichols, T. et al. Translational Data from AAV-Mediated Gene Therapy of Hemophilia B in Dogs. Hum Gene Ther Clin Dev, 2014; Calcedo, R. et al. Adeno-associated virus antibody profiles in newborns, children, and adolescents. Clin Vaccine Immunol 18, 1586-1588, 2011). However, the majority of DMD patients are typically diagnosed after the age of two, by which time massive muscle fiber degeneration, necrosis with mononuclear cell invasion, and increased fiber size variability have already occurred (Yiu, E. M. & Kornberg, A. J. Duchenne muscular dystrophy. Journal of paediatrics and child health 51, 759-764, 2015). To explore the feasibility of our approach in young boys with DMD, two juvenile GRMD dogs at 7.5 weeks of age (Hann and Beetle) were injected intravenously with AAV9-μUtrophin at a dose as high as 1.25×10¹⁴ vg/kg at time of injection, during transient use of an anti-inflammatory dose of prednisone, 1 mg/kg daily (Liu, J. M. et al. Effects of prednisone in canine muscular dystrophy. Muscle Nerve 30, 767-773, 2004) (FIG. 8A). Immunostaining of muscle biopsies, taken four weeks post-injection, showed homogeneous sarcolemmal expression of μUtrophin (FIG. 9A and data not shown), suppression of native utrophin (FIG. 9A), as well as rescue of the DGC (FIG. 9B). This was further confirmed by western blot (FIG. 9D).

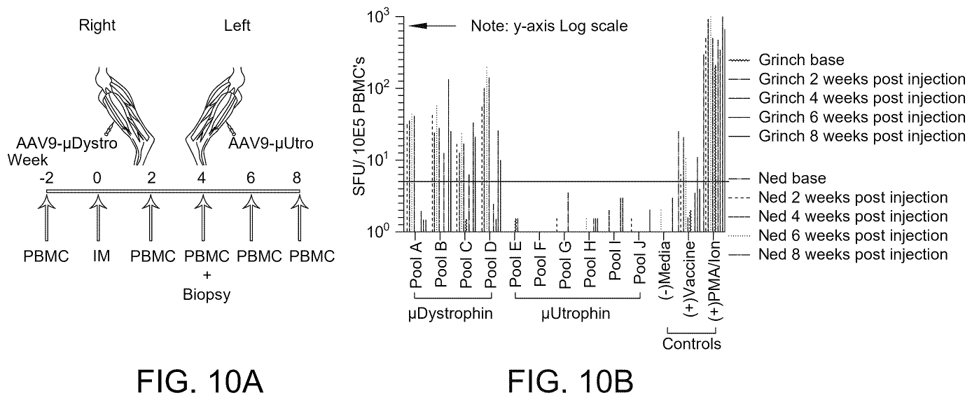

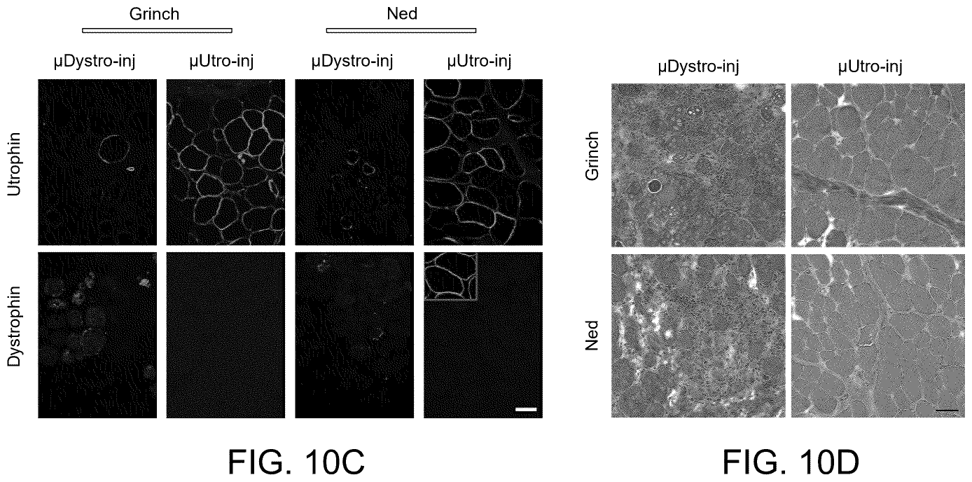

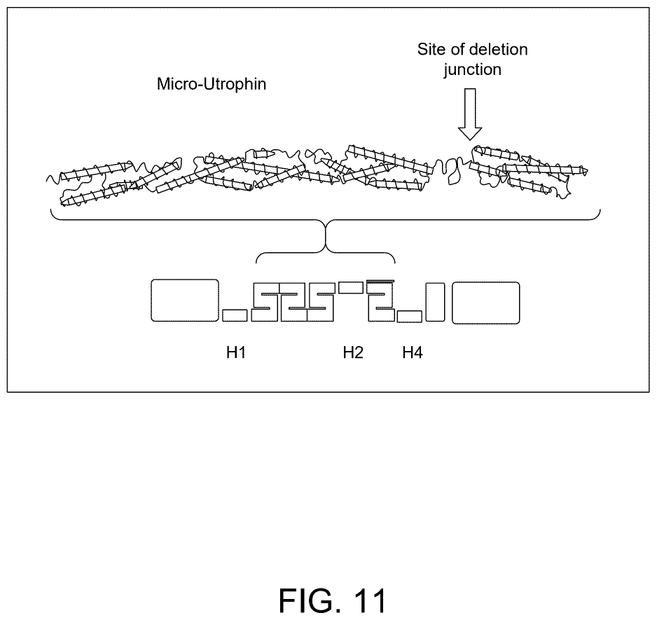

Histopathological characterization of limb muscle from treated GRMD dogs demonstrates near-complete suppression of ongoing muscle injury, as evidenced in untreated age-matched controls by a high rate of myonecrotic fibers, excessive calcium accumulation (FIG. 8C and FIG. 8E), clustered regenerating muscle fibers (FIG. 8D and FIG. 8F), abundant inflammatory cell infiltration and fat infiltration (FIG. 10B and data not shown). Impressively, AAV9-μUtrophin treated dogs also showed nearly complete prevention of muscle degeneration and regeneration in masticatory muscles (FIG. 8B and data not shown), which are severely affected in untreated dogs because they express the uniquely powerful MYH16 myosin isoform (Stedman, H. H. et al. Myosin gene mutation correlates with anatomical changes in the human lineage. Nature 428, 415-418 (2004); Toniolo, L. et al. Masticatory myosin unveiled: first determination of contractile parameters of muscle fibers from carnivore jaw muscles. Am J Physiol Cell Physiol 295, C1535-1542 (2008)). Further western blot analysis at necropsy (3.5 months of age) showed persistent widespread expression of μUtrophin in skeletal and cardiac muscle (FIG. 9C). Consistent with our previous GRMD neonatal dog studies, interferon-γ ELISpot assays revealed no signal above background against μUtrophin (data not shown), Furthermore, no signs of severe acute toxicity were seen, in contrast to previous studies in GRMD dogs and non-human primates (Kornegay, J. N. et al. Widespread muscle expression of an AAV9 human mini-dystrophin vector after intravenous injection in neonatal dystrophin-deficient dogs. Mol Ther 18, 1501-1508 (2010); Hinderer, C. et al. Severe Toxicity in Nonhuman Primates and Piglets Following High-Dose Intravenous Administration of an Adeno-Associated Virus Vector Expressing Human SMN. Hum Gene Ther, (2018); Hordeaux, J. et al. The Neurotropic Properties of AAV-PHP.B Are Limited to C57BL/6J Mice. Mol Ther, (2018)). Importantly, an 80% drop in serum CK levels was measured 1 week post-infusion with AAV9-μUtrophin in both dogs (FIG. 8G), a finding that is consistent with observed histological improvements. In order to achieve durable myoprotection throughout muscle growth from infancy to skeletal maturity, dystrophic dogs and boys with DMD may require systemic administration of AAV vector at doses proportional to those required in mdx mouse pups to maintain robust, homogeneous expression in the most severely affected muscle, the diaphragm (Stedman, H. H. et al. The mdx mouse diaphragm reproduces the degenerative changes of Duchenne muscular dystrophy. Nature 352, 536-539, (1991)), e.g. 1×10¹⁵ vg/kg neonatal body weight (data not shown).

For the rigorous preclinical assessment of vector and/or transgene immunotoxicity, we took advantage of the unique German Shorthaired Pointer (GSHPMD) deletional-null canine model (Schatzberg, S. J. et al. Molecular analysis of a spontaneous dystrophin ‘knockout’ dog. Neuromuscul Disord 9, 289-295. (1999); VanBelzen, D. J. et al. Mechanism of Deletion Removing All Dystrophin Exons in a Canine Model for DMD Implicates Concerted Evolution of X Chromosome Pseudogenes. Mol Ther Methods Clin Dev 4, 62-71, (2017)). The GSHPMD provides a superior platform for the study of central tolerance, since alternative splicing in the GRMD model allows detectable read-through of near-full-length dystrophin at potentially tolerizing levels, as might be expected to facilitate previously demonstrated therapeutic, long term body-wide expression of AAV-encoded canine microdystrophins (Schatzberg, S. J. et al. Alternative dystrophin gene transcripts in golden retriever muscular dystrophy. Muscle Nerve 21, 991-998. (1998); Yue, Y. et al. Safe and bodywide muscle transduction in young adult Duchenne muscular dystrophy dogs with adeno-associated virus. Hum Mol Genet 24, 5880-5890, (2015); Le Guiner, C. et al. Long-term microdystrophin gene therapy is effective in a canine model of Duchenne muscular dystrophy. Nat Commun 8, 16105, (2017)). Adult GSHPMD dogs (Ned and Grinch) each received equivalent doses (2×10¹² vg/kg) of both AAV9-μDystrophin and AAV9-μUtrophin via intramuscular injection into contralateral tibialis compartments (FIG. 10A). Interferon-Y detection via ELISPOT revealed the presence of a strong systemic cell-mediated immune response against u Dystrophin, as early as 2 weeks post-injection, but not to μUtrophin despite expression from the constitutive CMV promoter, indicating the strength of central immunological tolerance (FIG. 10B). Immunostaining of muscle biopsies collected 4 weeks post-injection revealed persistent expression of μUtrophin, but only sparse amounts of μDystrophin (FIG. 10C). H&E showed severe inflammation and mononuclear cell infiltration on the μDystrophin-injected side compared to their virtual absence on the μUtrophin-injected side (FIG. 10D and data not shown). These findings indicate that the observed immune response is driven by μDystrophin and not the vector capsid, as equivalent doses of vector were injected to both limbs.

In summary, after leveraging a comparative phylogenomic approach to identify evolutionary constraints, we have reverse-engineered a highly therapeutic, 3.5 kb synthetic transgene for safe systemic delivery to muscles of murine and canine models for DMD. Our blinded studies reveal surprisingly complete myoprotection as long as the initial level of gene delivery is sufficient to accommodate subsequent muscle growth. Taken together, these findings may refocus the field towards the use of a functionally optimized, non-immunogenic Utrophin-based gene therapy approach as a treatment for Duchenne Muscular Dystrophy.

### B. Materials and Methods

a. Bioinformatics and Phylogenetic Analysis

Publicly available genomic DNA sequences for species listed in figure legends and manuscript text were queried by multiple blast algorithms, in particular tBLASTn26,27, to identify the coding sequences homologous to the full-length human dystrophin and utrophin. For most species, supporting evidence from mRNA sequences was available to define intron/exon boundaries. Where such evidence was lacking, FGENESH+ (Softberry) was used with organism specific gene-finding parameters and Hidden Markov Model plus similar protein-based gene prediction to identify putative coding sequences from assembled contigs. As an internal test of this approach, virtually all transcriptome-defined coding sequences were properly identified by the FGENESH+ program28. We recognize that these mRNA and protein sequence files are missing sporadic exons in indeterminate regions of publicly available genomic DNA. HMMER (hmmer.janelia.org/search/hmmscan) was used with E-value defined cut-offs to define protein coding domains matching Hidden Markov Models for calponin homology, spectrin-like repeat, WW, EF hand, and ZZ domains. All deduced peptide sequence files were aligned by ClustalW using the default settings in MacVector version 13.5.1: Gonnet Series Matrix with parameters for pairwise alignment open gap penalty of 10, extend gap penalty of 0.1 and parameters for multiple alignment open gap series 10, extend gap penalty of 0.2 and delay divergence 30%. Phylogenetic reconstructions were generated with both full length and truncated sequences using the neighbor-joining tree building method with ties in trees resolved randomly and distances Poisson-corrected with gaps distributed proportionally or ignored to establish whether the choice impacted on tree topology. In all such cases, tree topology was insensitive to the management of gaps when the “best tree” mode was selected. Alternative use of the bootstrap mode, with 10000 replications, confirmed all nodes in the distance phylograms. Protein and DNA matrix analyses were based on the pam250 scoring matrix. Abbreviations used: Purple sea urchin, Strongylocentrotus purpuratus, S. pur; Amphioxus, Branchiostoma floridae, B. flo; Elephant shark, Callorhinchus milii, C. mil; Chinese alligator, Alligator sinensis, A. sin; Mouse, Mus musculus, M. mus; Dog, Canis familiaris, C. fam; Human, Homo sapiens, H. sap; Carolina anole, Anolis carolinensis, A. car; common chimpanzee, Pan troglodytes, P. tro; duck-billed platypus, Ornithorhynchidae anatinus, O. ana.; Japanese pufferfish, Takifugu rubripes, T. rub; tropical clawed frog, Xenopus tropicalis, X. tro; D—dystrophin; U—utrophin.

b. Microutrophin Transgene Design and Vector Production

Based on phylogenomic analysis of sequence conservation and genotype-phenotype correlations among BMD/DMD patients, we modeled in silico the spectrum of AAV-encodable miniaturized utrophins that preserve the calponin homology domains, the first three and last three spectrin-like repeats, and the combination of the WW, EF hand, and ZZ domains. To preserve amino acid side chain interactions between interhelical loops of adjacent spectrin-like repeats, we focused on only the subset of μUtrophin in which either the first or last three repeats were preserved intact. To minimize immunogenicity, we considered only those μUtrophin that could be created by the combination of a single internal deletion and a C-terminal truncation. Although the spectrin-like repeats are homologous to a consensus sequence, the divergence is such that no splice could be found between identical decapeptides in any of the mammalian utrophins. Thus, we used profile Hidden Markov Models, as implemented online at hmmer.janelia.org/search/hmmscan, to define and annotate spectrin-like triple helical repeat boundaries in the full length canine utrophin sequence (3456 aa, XP_005615306). We used transgenes encoding proteins matching the canine and human proteins in neonatal mice, in which antigen-specific tolerance is easily induced by intraperitoneal injection of AAV45, but only the canine versions in neonatal and older dogs in which tolerance is anticipated to require earlier prenatal exposure to the isogenic native protein during immune ontogeny (Davey, M. G. et al. Induction of Immune Tolerance to Foreign Protein via Adeno-Associated Viral Vector Gene Transfer in Mid-Gestation Fetal Sheep. PLOS One 12, e0171132, (2017)). The μUtrophin transgene was designed to contain the actin binding domain, triple helical repeats 1-3 and 22, a disordered, proline-rich region approximating that previously identified as “hinge” 2, and the C-terminal WW, EF hand, and ZZ domains, thus creating a recombinant protein designed to match the canine and human utrophin sequence with the exception of a single splice site at the deletion junction, thereby minimizing potential immunogenicity in dystrophin deficient dogs and ultimately humans relative to previously reported transgenes (Wang, B., et al. Adeno-associated virus vector carrying human minidystrophin genes effectively ameliorates muscular dystrophy in mdx mouse model. Proc Natl Acad Sci USA 97, 13714-13719. (2000); Harper, S. Q. et al. Modular flexibility of dystrophin: implications for gene therapy of Duchenne muscular dystrophy. Nat Med 8, 253-261. (2002); Gregorevic, P. et al. Systemic delivery of genes to striated muscles using adeno-associated viral vectors. Nat Med 10, 828-834 (2004); Gregorevic, P. et al. rAAV6-microdystrophin preserves muscle function and extends lifespan in severely dystrophic mice. Nat Med 12, 787-789 (2006); and Odom, G. L., et al. Microutrophin delivery through rAAV6 increases lifespan and improves muscle function in dystrophic dystrophin/utrophin-deficient mice. Mol Ther 16, 1539-1545 (2008)). The coding sequence chosen for use in our studies was selected as the highest-expressing candidate of a pool of cDNAs that were optimized and synthesized by competing biotech companies (GeneArt and DNA 2.0). Expression was determined by immunofluorescence staining and western blotting following electroporation of 50 μg DNA in tibialis anterior muscle of mdx mice. The synthetic coding sequence chosen for further use was found to drive expression approximately 30-fold higher in in vitro and in vivo assays than the wild type canine cDNA sequence encoding a recombinant protein of identical primary structure. A notable difference between the best synthetic cDNA and the wild type is the level of codon bias, with only the optimized synthetic cDNA closely matching the extreme bias of the mammalian myosin heavy chains (e.g. 154 CTG leucines, 0 TTA leucines). The synthetic canine μUtrophin cDNA was subcloned into an AAV2 expression vector cassette driven by an 833 bp fragment of the CMV immediate early enhancer/promoter or a synthetic promoter spc5-12 (CMV and SP, respectively). AAV9 vectors were generated and purified by the University of Pennsylvania preclinical vector core using the triple transfection method in HEK 293 cells as previously described (Vandenberghe, L. H. et al. Efficient serotype-dependent release of functional vector into the culture medium during adeno-associated virus manufacturing. Hum Gene Ther 21, 1251-1257, doi: 10.1089/hum.2010.107 (2010)). Vector preparations were assayed for quality, purity and endotoxin levels prior to pooling for injection of 2×10¹¹ AAV9 μUtrophin vg into the tibialis anterior muscles of mdx mice (Lock, M., et al. Analysis of particle content of recombinant adenoassociated virus serotype 8 vectors by ion-exchange chromatography. Hum Gene Ther Methods, 23, 56-64, doi: 10.1089/hgtb.2011.217 [pii]).

### c. Animals—General

The Animal Care and Use Committee of the A&M University and the University of Pennsylvania approved all animal experiment protocols in mice and dogs.

### d. Murine Model Vector Administration

Mouse strains C57BL/10SnJ and mdx were purchased from the Jackson laboratory (Bar Harbor, ME). This study involved 23 C57BL/10SnJ mice and 30 mdx mice, all injected at 9±2 days of age. Prior to receiving intraperitoneal injection of AAV9 μUtrophin or Phosphate Buffered Saline (PBS), individual pups were toe-tattooed with the Aramis Micro tattoo kit (Ketchum Manufacturing Inc, Canada) and randomly assigned to different dosage groups. Investigators were blinded during all injections and tissue harvesting. Based on this protocol, C57BL/10SnJ and mdx pups were injected with 50-250 μl of either PBS as negative control or AAV9 μUtrophin diluted in PBS via 32-gauge insulin syringe. Prior to injection, each mouse was weighed. After vector administration, all mice were returned to their litters and separated after weaning.

### e. Murine Model Tissue Procurement and Storage

At approximately 8 weeks of age, mdx and C57BL/10SnJ mice underwent CO₂ euthanasia in accordance with the institutional policy. The heart, tibialis anterior, gastrocnemius, quadriceps, triceps, abdominal, diaphragm, temporalis muscles, and liver were harvested and further processed; others were stored but not utilized based on studies showing <100-fold lower off-target gene expression in AAV9 in mice Zincarelli, C. et al. Analysis of AAV serotypes 1-9 mediated gene expression and tropism in mice after systemic injection. Mol Ther 16, 1073-1080 (2008)). Designated histological tissue samples were placed in OCT (Tissue-Tek) containing embedding molds (Richard-Allan Scientific) and rapidly frozen in liquid nitrogen-cooled isopentane. Additional designated biological tissue samples were placed in tissue containers and rapidly frozen in liquid nitrogen. All the specimens were stored at −80° C. Cryosections of 5-7 μm thickness were cut on a cryostat (Microm HM550, Thermo Scientific, USA) at −25° C. and mounted on glass slides (Superfrost Plus, Fisher Scientific, USA).

### f. Basic Histology—Haematoxylin and Eosin Staining (H&E) and Alizarin Red Staining (ARS)

7 μm thick cross sections were air dried for 15 min at room temperature. Then the slides were stained with Harris' hematoxylin dye for 2.5 minutes, rinsed in distilled water, dipped in 0.1% acetic acid for 15 seconds, followed by a repeat rinse in tap water for 4 mins and counterstaining with 1% eosin for 1 min. As a final step, the slides were dehydrated in ethanol three times, 2 mins each. Representative, non-overlapping high-powered fields (HPF) were photographed for scoring (data now shown). Alizarin red staining was also carried out on 7 μm thick cross sections. After 10 min fixation by 10% buffered formalin phosphate, the sections were washed 3×5 min with PBS and incubated with alizarin red dye for 15 min at room temperature. After this procedure, the slides were washed in ethanol 3 times and mounted by cytoseal 60 (Thermo Scientific).

### g. Morphometric Analysis

Three groups of mice, C57BL/Sn10J (control), and mdx randomized to either injection with PBS or AAV9 μUtrophin, were studied by investigators blinded to specimen identification. Four to five randomly chosen areas from each tibialis anterior, gastrocnemius, quadriceps, triceps, temporalis and abdominal muscles muscle were stained with H&E and screened with light microscope for quantification of centrally nucleated myofibers. Areas at the myotendonous junctions were excluded from the measurements as they are rich in centrally nucleated fibers in both mdx and controls. In total, 11,649 fibers were evaluated.

### h. Immunofluorescence Staining Procedures: N-Terminal and C-Terminal Utrophin Double Staining

Sections from all muscle specimens were processed for utrophin immunostaining by using both N-terminal polyclonal (against full-length and recombinant utrophin) as well as C-terminal monoclonal (against full-length utrophin) antibodies. After initial incubation for 20 mins in a 1% solution of Triton X-100 (Roche Diagnostics GmbH, Mannheim, Germany) diluted in 0.01M PBS (Roche Diagnostics GmbH, Mannheim, Germany), the specimens were rinsed three times in PBS for 5 minutes each (3×5 mins). Sections were then incubated in 5% normal donkey serum for 15 mins, followed by an incubation with N-terminal utrophin antibody (N-19, sc-7460, goat polyclonal IgG, Santa Cruz, CA, USA, dilution 1:50) for 60 min at 37° C. After a second cycle 3×5 min PBS wash, the slides were incubated with 5% normal donkey serum for 15 mins in room temperature. The prepared sections were then incubated in donkey anti-goat IgG-FITC (sc-2024, Santa Cruz, CA, USA, dilution 1:300) for 30 min at 37° C. Following a third PBS wash for 3×5 mins, the sections were first incubated with 10% normal goat serum (Invitrogen, Scotland, UK) for 15 mins and then with the C-terminal utrophin antibody (MANCHO7, mouse monoclonal IgG2a, Santa Cruz, CA, USA, dilution 1:25) at 37° C. for 60 mins. After a PBS wash for 3×5 mins and incubation with 10% normal goat serum, the sections were incubated in goat anti-mouse IgG2a-Alexa Fluor® 594 (A-21140, Life Technologies, USA, dilution 1:300) for 30 min at 37° C. The sections were again washed in PBS for 3×5 mins and mounted in Vectashield Mounting Medium (H-1000) (Vector Laboratories, CA, USA) or Mounting Medium with DAPI (H-1500) (Vector Laboratories). Photographs were taken with Leica DM6000B microscope (Leica, Germany).

### i. Immunofluorescence Staining Procedures: Double Immunofluorescence Staining for γ-Sarcoglycan/Laminin, MuRF-1/Laminin and MyHC-Embryonic/Laminin

The staining procedures for these proteins followed the same protocol as described previously (Song, Y., et al. Effects on contralateral muscles after unilateral electrical muscle stimulation and exercise. PloS one 7, e52230, doi: 10.1371/journal.pone.0052230 (2012)). Rabbit anti-γ-sarcoglycan (NBP1-59744, Novus Biologicals, Littleton, CO), and MURF1 (NBP1-31207, Novus Biologicals, Littleton, CO) polyclonal antibodies were used at a dilution of 1:50 in PBS with Bovine Serum Albumin (BSA). MyHC-embryonic monoclonal antibody (F1.652) (Developmental studies, Hybridoma Bank, Iowa, USA) was used at a dilution of 1:50-1:100 in PBS. Laminin chicken polyclonal antibody (ab14055-50, Abcam, Cambridge, MA, USA) at a dilution of 1:500-1:1000 together with second goat anti-chicken IgY (TR) antibody (ab7116, Abcam, Cambridge, MA, USA, dilution 1:300) were applied to identify muscle fibers. MYH16 rabbit polyclonal antibody peptide sequence was generated using the human canine sequence of MYH16's “Loop 2” region. Peptide Sequence: LLALLFKEEEAPAGS

### j. TUNEL Assay

Sections were initially fixed in 10% buffered formalin phosphate (Fisher Scientific, USA) for 20 min. In situ nick end labeling of fragmented DNA was then performed using TACS 2 TdT Fluorescein apoptosis detection kit (Trevigen, Gaithersburg, MD, USA), as described by the manufacturer's instruction.

### k. Serum Creatine Kinase (CK) Assay

Blood serum was collected via venipuncture of the submandibular vein using a 5 mm animal lancet (Goldenrod Animal Lancet, Braintree Scientific, Inc, Braintree, MA). Total of 150 μL were collected in a heparinized blood collection tube (Terumo, Catalog Number: TMLH). The mice were carefully monitored for 30 minutes post blood withdraw to observe for potential signs of distress. CK levels were determined by the Clinical Pathology Laboratory at the Matthew J. Ryan Veterinary Hospital of The University of Pennsylvania.

### l. Ex Vivo Evaluation of EDL Muscle Contractile Properties

Ex vivo assessments was performed by Muscle Physiology Assessment Core of The University of Pennsylvania. The physiological properties, including isometric twitch force, isometric tetanic force, and force drop after ECCs, were quantified on freshly isolated EDL muscles from 2 months old mdx mice using an Aurora Mouse 1200A System equipped with Dynamic Muscle Control v. 5.3 software. All of these mice had undergone in vivo force grip testing 24 hours prior to euthanasia an ex vivo testing. EDL muscles were maintained in constantly oxygenated Ringer's solution (100 mM NaCl, 4.7 mM KCl, 3.4 mM CaCl₂), 1.2 mM KH2PO4, 1.2 mM MgSO4, 25 mM HEPES and 5.5 mM D-glucose) at 24° C. The twitch stimulation protocol applied was a single stimulus with a duration of 0.2 ms. For measuring tetanic maximal force generation, the same stimulus was repeated at a frequency of 120 Hz for 500 ms. Five min were allowed between two tetanic contractions to ensure muscle recovery. Muscle length was adjusted to obtain the maximal twitch response and this length was measured between the outermost visible tips of the myotendinous junctions and recorded as optimal length (L0). Muscle cross-sectional area (CSA) of EDL muscles were calculated by dividing the muscle mass by the product of the muscle density coefficient (1.06 g/cm³), muscle L0, and the fiber length coefficient (0.45 for EDL). Specific force was determined by normalizing the force to CSA.

After testing the isometric properties of EDL, a series of five eccentric contractions (ECCs—one every five minutes) was applied in cycles beginning with repeated 500 ms isometric contractions followed by stretching the muscle by 10% of L₀ while administering a maximal tetanic stimulation. The reported absolute force for each ECC corresponds to the peak force during the isometric phase of the ECC.

### m. Vertical Activity & Grip Strength Test

Mice were carefully placed in the open field cage and their baseline vertical activity was determined for five minutes. Mice were then returned to their original cages and allowed to rest for three minutes. An axial force transducer was used to measure force (Vernier LabPro & Vernier Dual-Range Force Sensor+10N, Beaverton Oregon), while data were collected using the accompanying software (Logger Lite version 1.8.1). All experiments were performed by the same experimenter in a blinded fashion. To reduce chances of bias and to ensure the robustness of the blinded experiment we used the approach as described (Song, Y. et al. Suite of clinically relevant functional assays to address therapeutic efficacy and disease mechanism in the dystrophic mdx mouse. J Appl Physiol 122, 593-602, doi: 10.1152/japplphysiol.00776.2016 (2017)).

### n. Canine Model

Two groups of dogs were used for our experiment. The first group was bred in a colony at the A&M University and whelped at the University of Pennsylvania. This study involved five affected GRMD dogs and four age matched littermates including one wild type and three carrier females that served as a control group. All dystrophic dogs were identified by elevated serum creatine phosphokinase (CPK) levels and genotyped by PCR assay. All pups were randomly assigned to treatment groups, and the investigators were kept blinded during clinical and histological assessment. The pups were injected with AAV9 μUtrophin at 6-10 days of age at the dose of 1.0×10¹³ vg/kg and 1.0×10^{13.5} vg/kg via external jugular vein approach. Two pups were injected with low dose AAV9 μUtrophin, two were injected with high dose, and the remaining one was injected with saline only. Each dog was weighed daily for the first 6 weeks and weekly thereafter.

The second canine group involved two GRMD dogs at the age of approximately 7.5 weeks. Three days prior to vector administration, the dogs were placed on oral prednisolone 1 mg/kg regimen for 25 days. AAV9 μUtrophin was injected using the same approach at two different single doses of 1.25×10¹⁴ vg/kg and 5.0×10¹³ vg/kg respectively. The dogs were randomly assigned to dose and the investigators were kept blinded during the clinical and histological assessments.

Deletional-null German Short Hairpointer (GSHPMD) dogs were bred and housed in Texas A&M University. Two seven year old affected GSHPMD dogs, Ned and Grinch, weighed 21 kg and 24.2 kg respectively, Each dog received five intramuscular injections of AAV9-μDystrophin (Right) and AAV9-μUtrophin (Left), with a total equivalent dose of 1.0×10¹² vg/kg, into their tibialis anterior compartment. All five injection sites were tattooed, allowing us to pinpoint the injection sites for muscle biopsies 4 weeks post-injection. Peripheral blood was collected pre-, 2, 4, 6, 8 weeks post-injection in order to collect Peripheral blood mononuclear cells (PBMCs).

### o. Canine Tissue Procurement and Storage

The first group of GRMD dogs underwent needle biopsy of the cranial sartorius, vastus lateralis, and triceps brachii muscles at approximately 6 weeks post vector injection. The specimens were stored with a set of blinding codes to prevent bias during analysis and interpretation. Biopsies were obtained through a spring loaded 14-gauge needle trocar, thereby significantly minimizing the post-procedural pain in the animals. Muscles biopsies were then snap-frozen in liquid nitrogen-cooled isopentane, embedded in OCT medium (Sakuru, USA) and stored at −80° C. Blinding codes were broken after tissue analysis by individuals not associated with authorship of this study. One month after vector exposure the second group of injected dogs underwent open muscle biopsy of the same muscles. Seven weeks post vector administration these dogs were euthanized and the harvested tissue was cryopreserved in the same way.

### p. Canine Histological Analysis

Transversely cut 7 μm serial sections were used for bright-field microscopy analysis and immunofluorescence (IF) staining to examine microutrophin expression and sarcoglycan rescue. Muscle sections were stained with H&E for bright-field microscopy and mounted with permount. For IF staining, sections were blocked in 5% donkey serum in PBS for 45 mins followed by incubation for 60 min at 37° C. using a 1:350 dilution of polyclonal goat anti-utrophin antibody (N-19, sc-7460, Santa Cruz, USA) and 1:250 dilution of monoclonal γ-sarcoglycan antibody (ab55683, Abcam, USA). The sections were then rinsed three times in PBS and incubated for 45 mins in Alexa488 donkey anti-goat secondary antibody or Alexa540 donkey anti-mouse secondary antibody at a dilution of 1:1000. The slides were washed twice with PBS for 5 mins followed by single wash of water and mounted with fade resistant mounting media containing DAPI (H-1500, Vector Labs). Images were captured at the same setting and processed via identical way by setting the limits and gain throughout to avoid any disparity in IF images using an Olympus B-65 fluorescent microscope. Minimal Feret's diameter and the coefficients of variance were calculated according to TREAT_NMD protocol DMD_M.1.2.001 as updated Jan. 28, 2014.

### q. Immunoblot Analysis

Immunoblot analysis was carried out by loading 20-40 μg/lane of whole cell or whole muscle lysate on 10% sodium dodecyl sulfate-polyacrylamide gel. Protein was transferred to a polyvinylidene difluoride membrane. Microutrophin was detected by goat polyclonal antibody against the N-terminal epitope at 1:500 dilution (N-19, sc-7460, Santa Cruz, USA) and a secondary antibody, a donkey anti-goat antibody conjugated with horseradish peroxidase (Sigma-Aldrich) at 1:5000 dilution. Protein detection and quantification was performed using the Odyssey infrared imaging system (LI-COR). Gamma-sarcolgycan was detected by a mouse monoclonal antibody (Vector Labs VP-G803) and a donkey, anti-mouse HRP conjugated secondary antibody (Santa Cruz Biotechnology).

### r. Detection of Neutralizing Anti-AAV Antibodies

To assess the humoral immune response against AAV 9 capsid proteins, blood sera were collected at the day of birth, then at weeks 4 and 8 via peripheral vein. HEK 293 cells were seeded in a 48-well plate at a density of 10⁵ cells/well in 200 μl DMEM containing 10% fetal bovine serum. The cells were cultured for 3-4 hours at 37° C. and allowed to adhere to the well.

AAV9-Green Fluorescence Protein (GFP) vector (1×10⁸ particles) was incubated with mice sera at serial dilution with PBS for two hours at 4° C. in a total volume of 25 μL. The mixture was then added to cells in a final volume of 200 μl which contained 4×10⁶ particles of AAV9 and incubated for 24 or 48 hours at 37° C. Cells expressing GFP were counted under a fluorescent microscope. The neutralizing antibody titer was calculated using the highest dilution where the percentage of GFP-positive cells was 50% less than control without sera.

### s. Evaluation of T-Cell Reactivity to Capsid Derived Peptides

Peripheral blood T cell responses to the novel AAV capsid antigens were quantified by IFN-γ ELISpot assay (Mingozzi, F. et al. AAV-1-mediated gene transfer to skeletal muscle in humans results in dosedependent activation of capsid-specific T cells. Blood 114, 2077-2086 (2009)). Briefly, peripheral blood mononuclear cells (PBMC) were isolated on Ficoll hypaque gradients and cultured with synthetic peptides (20 amino acids in length, overlapping by 10 residues) that spanned the VP1 capsid protein. To identify individual peptides within a pool that elicited IFN-γ activity, each peptide was present in two of the intersecting mapping sub-pools. After incubation at 37° C. for 36 hours, IFN-γ SFU were counted. Fewer than 10 SFU/well were observed with peptides from a control pool (enhanced GFP). Responses were considered positive when SFU exceeded 50/10⁶ PBMC in duplicate wells.

### t. Statistical Analysis

Informative group sizes for AAV vector injections in mdx mice were estimated based on one of the most sensitive and widely used histological assays available: the proportion of centrally nucleated muscle fibers from mice necropsied at 8 weeks of age. To maximize the statistical power for the number of animals used, we adopted the approach described by Aarts, et al. (Aarts, E., et al., A solution to dependency: using multilevel analysis to accommodate nested data. Nat Neurosci, 2014. 17 (4): p. 491-6), to accommodate dependency among the multiple high-powered fields (HPFs) within a mouse. Mixed effects models accounting for clustering within mouse, using an exchangeable correlation structure, were used to compare the three groups defined by genotype and treatment. Estimation of the intracluster correlation (ICC) from these models indicated a low value (<10%), suggesting a relatively high effective sample size despite the necessarily small numbers of mice per group (at least four). Other random-effects parameters calculated in this analysis include: the variance between clusters, σ²ᵤ; the variance within clusters, σ²ₑ; and the effective sample size, n_{eff}.

To characterize the distribution of minimum Feret diameter in wild type, treated, and untreated dystrophic dogs, dots representing individual measurements from representative HPFs were plotted. Due to the small number of dogs, this analysis is entirely descriptive. All analysis except the proportion of centrally nucleated muscle fibers were presented as mean±S.D. Statistical analysis was processed using Prism 7 software

(GraphPad). Statistical significance of p values is indicated in the figures: *p<0.05; **p<0.001; ***p<0.0001; n.s., not significant.

### Example 3—Systemic Gene Therapy for DMD Using AAV-Mediated Delivery of Nano-Utrophin and Nano-Dystrophin

### A. Human Nano-Utrophin Design

A major role of the rod domain is the longitudinal transmission of force, and the extraordinary length of dystrophin reflects the protein's evolutionary legacy. As a result of this insight, we have identified a way of addressing a central limitation of other approaches, and to design novel transgenes to achieve force transduction and myoprotection at levels indistinguishable from wild type.

Our analysis of sequences for orthologs and paralogs of the dystrophin genes and transcripts from a broad sampling of metazoan taxa led us to hypothesize that BMD in patients with frame-preserving deletions or duplications results from focal destabilization of the rod domain during mechanical loading. This hypothesis is supported by crystallographic data from dystrophin and other proteins containing triple helical repeat domains, bioinformatic analysis of evolutionary coupling among amino acids in adjacent triple helical repeats, and unpublished data from our studies of miniaturized recombinant proteins in mechanically loaded muscle. None of the recombinant mini-dystrophins reported to date have avoided the potential for a weakest link at the internal deletion site, as all of the design approaches used model the triple helical repeats as interchangeable, modular units. Despite its impressive phenotypic amelioration in animal models, even micro-Utrophin risks this limitation. To address this possibility and to further shorten the transgene, we have developed a model for a triple-spliced “nano-utrophin” (FIG. 4). We “cut” and “weld” the dystrophin and utrophin rod across the structurally most conserved three-dimensional planes of two non-adjacent triple helical repeats in order to preserve all the evolutionarily-coupled amino acid side chain interactions at the less conserved, loadbearing loop-to-loop interfaces between adjacent repeats. This is modeled in FIG. 4 using the domino metaphor. In the next section this is described in greater detail using graphical representations from molecular modeling. Evaluations are performed regarding the triple-spliced nano-utrophin, for potential improvement over the micro-utrophin to identify therapeutic transgenes through sequential testing in dystrophin deficient mice (immediate) and rats (better pathogenetic model for DMD fibrosis, degeneration, and weakness but requiring 10× more vector).

### B. Scalable Production of AAV for Systemic Delivery in DMD.

The AAV9 and Anc80 vectors utilized in our preclinical studies in dystrophic mice and dogs to date have been prepared in human HEK 293 cells by applying the triple plasmid transfection method. This technology relies on the growth of anchorage-dependent cells in rich culture media, with the input plasmids propagated at large scale in bacterial strains prior to transfection such that the end-product retains the potentially immunogenic bacterial DNA methylation patterns. AAV vector discovery and production using a wide range of capsids and platforms are performed including anchorage-dependent and suspended HEK 293 cells as well as baculovirus-infected Sf9 insect cells.

### C. Animal Models

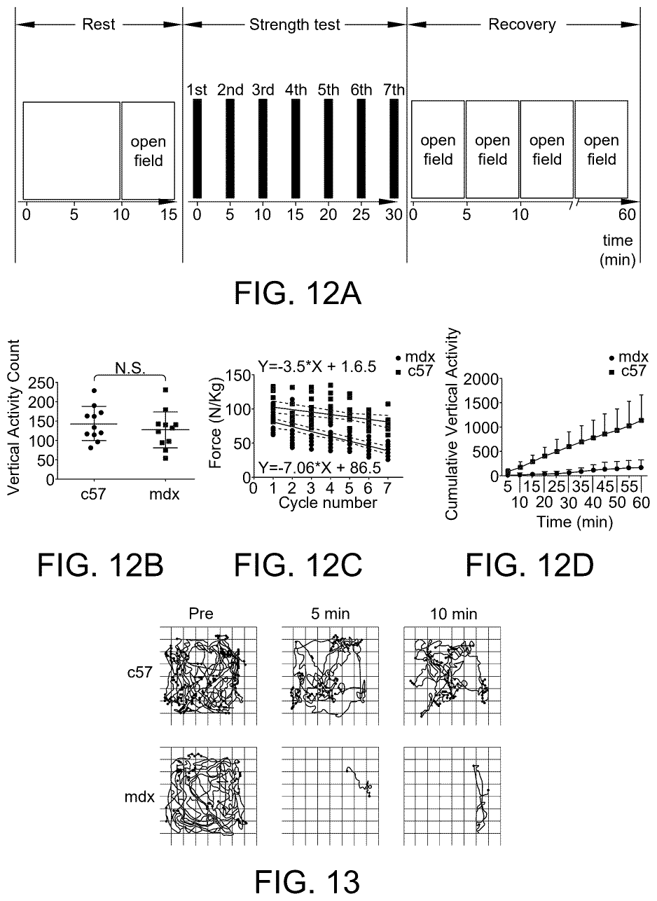

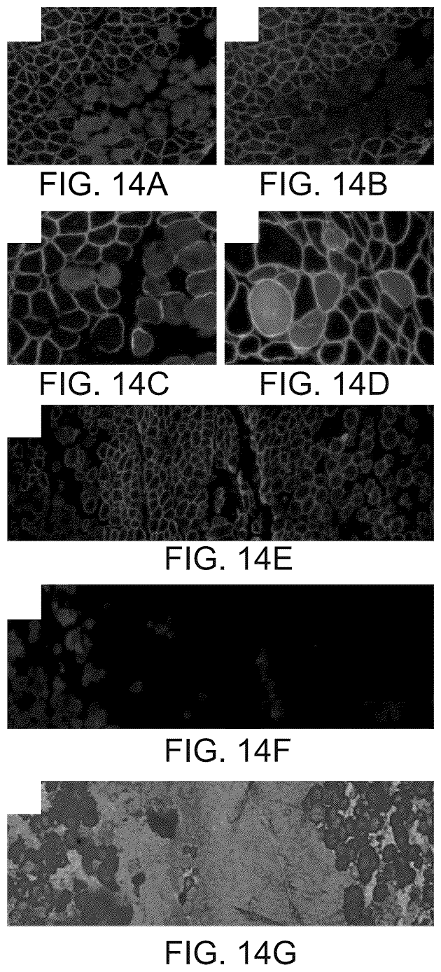

Rigorous, blinded tests of AAVμ-versus n-Utrophin systemic therapy are investigated using primary, secondary, and exploratory endpoints that inform the design and execution of future clinical trials. Two primary endpoints are selected: performance in the most sensitive of our published suite of integrative physiological assays in mdx mice (the hybrid limb force vertical activity test, see figures from (Song, Y., et al., Suite of Clinically Relevant Functional Assays to Address Therapeutic Efficacy and Disease Mechanism in the Dystrophic mdx Mouse. J Appl Physiol, 2016: p. jap 00776 2016), and the quantitative immunodetection of the N-terminal 25 kDa fragment of titin (Robertson, A. S., et al., Dramatic elevation in urinary amino terminal titin fragment excretion quantified by immunoassay in Duchenne muscular dystrophy patients and in dystrophin deficient rodents. Neuromuscul Disord, 2017. 27 (7): p. 635-645). See, e.g., FIG. 12A-12D and FIG. 13. The latter is a non-invasive, urine-based bioassay that has extraordinary dynamic range, uniquely reflects the systemic level of myonecrosis, and builds on the role of titin isoforms in myofibrillogenesis (Example 1). A major goal of this aim of the project is to quantitatively characterize the extent and durability of phenotypic amelioration in a cost-effective model that recapitulates the major pathological features of DMD. Studies in mdx mice are systematically extended to dystrophin-deficient rats because this latter model more closely resembles the hallmark pathological myo-degeneration and fibrosis seen in DMD with easily quantified indices of progressive muscle weakness and cardiomyopathy Robertson, A. S., et al., Dramatic elevation in urinary amino terminal titin fragment excretion quantified by immunoassay in Duchenne muscular dystrophy patients and in dystrophin deficient rodents. See, Petrof, B. J., et al., Dystrophin protects the sarcolemma from stresses developed during muscle contraction. Proc Natl Acad Sci, 1993. 90: p. 3710-14; Neuromuscul Disord, 2017. 27 (7): p. 635-645; Larcher, T., et al., Characterization of dystrophin deficient rats: a new model for Duchenne muscular dystrophy. PLOS One, 2014. 9 (10): p. e110371; Nakamura, K., et al., Generation of muscular dystrophy model rats with a CRISPR/Cas system. Sci Rep, 2014. 4: p. 5635; Stedman, H. H., et al., The mdx mouse diaphragm reproduces the degenerative changes of Duchenne muscular dystrophy. Nature, 1991. 352 (6335): p. 536-9; Shrager, J. B., et al., The mdx mouse and mdx diaphragm implications for the pathogenesis of Duchenne Muscular Dystrophy., in Neuromuscular Development and Disease, A. M. Kelly and H. M. Blau, Editors. 1992, Raven Press, Ltd.: New York. p. 317-328; Krupnick, A. S., et al., Inspiratory loading does not accelerate dystrophy in mdx mouse diaphragm: implications for regenerative therapy. J Appl Physiol, 2003. 94 (2): p. 411-9; and Song, Y., et al., Suite of clinically relevant functional assays to address therapeutic efficacy and disease mechanism in the dystrophic mdx mouse. J Appl Physiol, 2017. 122 (3): p. 593-602. Several canine disease models (Cooper, B. J., et al., The homologue of the Duchenne locus is defective in X-linked muscular dystrophy of dogs. Nature, 1988. 334 (6178): p. 154-6; Smith, B. F., et al., Molecular basis of canine muscle type phosphofructokinase deficiency. J Biol Chem, 1996. 271 (33): p. 20070-4; Bridges, C. R., et al., Global cardiac-specific transgene expression using cardiopulmonary bypass with cardiac isolation. Ann Thorac Surg, 2002. 73 (6): p. 1939-46; Arruda, V. R., et al., Regional intravascular delivery of AAV-2-F.IX to skeletal muscle achieves long-term correction of hemophilia B in a large animal model. Blood, 2004. Epub ahead of print; Arruda, V. R., et al., Peripheral transvenular delivery of adeno-associated viral vectors to skeletal muscle as a novel therapy for hemophilia B. Blood, 2010. 115 (23): p. 4678-88; Mead, A. F., et al., Diaphragm remodeling and compensatory respiratory mechanics in a canine model of Duchenne muscular dystrophy. J Appl Physiol (1985), 2014. 116 (7): p. 807-15; and Su, L. T., et al., Uniform scale-independent gene transfer to striated muscle after transvenular extravasation of vector. Circulation, 2005. 112 (12): p. 1780-8), may be used, as well as the hamster model for LGMD (e.g. histological assays in FIG. 14A-FIG. 14G, from Greelish, et al, Nature Medicine, (Greelish, J. P., et al., Stable restoration of the sarcoglycan complex in dystrophic muscle perfused with histamine and a recombinant adeno-associated viral vector. Nat Med, 1999. 5 (4): p. 439-43), which formed an essential part of the background for the first phase I clinical trial of gene therapy using AAV vectors in human muscular dystrophy: Stedman, et al, Human Gene Therapy. Stedman, H., et al., Phase I clinical trial utilizing gene therapy for limb girdle muscular dystrophy: alpha-, beta-, gamma-, or delta-sarcoglycan gene delivered with intramuscular instillations of adeno-associated vectors. Hum Gene Ther, 2000. 11 (5): p. 777-90).

### D. Product Profile

Target Product Profile for AAVuUtrophin or AAVnUtrophin.

| Product Targets | Minimum Acceptable Result | Ideal Results |
|---|---|---|
| Primary Product | Symptoms and signs of extremity muscle | Symptoms and signs of locomotive, |
| Indication | dysfunction in Duchenne and Becker | respiratory, and cardiac muscle dysfunction |
| | Muscular Dystrophy resulting from any | in Duchenne and Becker Muscular |
| | dystrophin gene mutation | Dystrophy resulting from any dystrophin |
| | | gene mutation |
| Patient Population | Ambulatory or post-ambulatory patients | Presymptomatic infants, symptomatic boys |
| | with progressive symptoms and signs of | and young adults with DMD/BMD |
| | DMD | |
| Treatment Duration | Single dose | Single dose |
| Delivery Mode | IV | IV |
| Dosage Form | Frozen suspension of recombinant AAV | Frozen suspension of recombinant AAV |
| | vector, for single use after thawing | vector, for single use after thawing |
| Regimen | Infusion-long catheter in antecubetal vein | Infusion-long catheter in antecubetal vein |
| | for systemic infusion; cephalic and/or | for systemic infusion; cephalic and/or |
| | saphenous vein distal to tourniquet for | saphenous vein distal to tourniquet for |
| | extremity | extremity |
| Efficacy | Multiyear slowing in rate of progression of | Complete prevention of symptoms of |
| | functional loss in injected extremities | locomotive, respiratory, and cardiac disease |
| | | associated with DMD/BMD, based on |
| | | confirmed early pre-symptomatic diagnosis |
| Risk/Side Effect | Devoid of long-term immunotoxicity from | Devoid of long-term immunotoxicity from |
| | encoded recombinant protein. May require | encoded recombinant protein. May require |
| | transient immunsuppression to minimize | transient immunsuppression to miniize risk |
| | risk of immune response to vector capsid. | of immune response to vector capsid. |
| | Readministration may be prohibited by the | Readministration may be prohibited by the |
| | development of antibodies against the | development of antibodies against the |
| | vector's protein capsid. Applicable maximal | vector's protein capsid. |
| | dose may be limited by the risk of toxicity | |
| | involving inmate immunity and/or | |
| | hepatotoxicity. | |
| Therapeutic modality | Myotropic adeno-asociated virus vector | Myotropic adeno-asociated virus vector |
| | encoding artificially spliced isoform of | encoding artificially spliced isoform of |
| | utrophin | utrophin |

The chart above depicts the target product profiles for AAVμUtrophin or AAVnUtrophin, Our current understanding of the progressive loss and fibro-fatty 5 replacement of striated myocytes in DMD suggests that significant reversal of the disease in older subjects may be limited, but prevention of further myocyte loss may be possible at any stage following the onset of recombinant μ- or n-Utrophin expression. Experiments are performed directed primarily at dystrophin-deficient mice and rats to inform expectations for ideal parameters in DMD patients treated in infancy. Transduction with sufficient AAVμ- or n-Utrophin in infancy to normalize sarcoglycan expression throughout growth to skeletal maturity may allow for relatively normal growth of muscle and hence normal maturational increase in strength. At least four factors may limit therapeutic benefit in older patients: 1) the extent of irreversible myocyte loss prior to treatment, 2) the extent to which fibro-fatty replacement of muscle impairs vector delivery and myocyte transduction, 3) the maturational decrease in endothelial permeability to vector, potentially requiring forced extravasation from the vascular lumen distal to a tourniquet in older patients, and 4) the anticipated increase in natural exposure to AAV viruses with advancing patient age, thereby increasing the proportion with both high titer antibodies to multiple AAV serotypes and memory T cells to conserved AAV capsid-derived peptides. Dose-limiting toxicity may involve the innate immune system and/or the liver.

E. Experimental and Theoretical Basis for μ- or n-Utrophin Substitution in Dystrophin Deficiency

Utrophin was originally discovered on the basis of its coding sequence homology to dystrophin (Love, D. R., et al., An autosomal transcript in skeletal muscle with homology to dystrophin. Nature, 1989. 339 (6219): p. 55-8). We have recently reconstructed the evolutionary histories of dystrophin and utrophin on the basis of publicly available whole genome sequences from a wide range of taxons. Two observations are relevant: 1) the “donor” gene for the rod-like domain of both proteins had at least 21 tandem spectrin-like repeat domains long before it was joined, by partial gene duplication, to the Dp71-like domain prior to the emergence of striated muscle, 2) separate genes for utrophin and dystrophin were fixed after the divergence of cephalochordates along the lineage leading to a common ancestor of jawless and jawed vertebrates and before the evolutionary appearance of oligodendrocytes (Putnam, N. H., et al., The amphioxus genome and the evolution of the chordate karyotype. Nature, 2008. 453 (7198): p. 1064-71; and Smith, J. J., et al., Sequencing of the sea lamprey (Petromyzon marinus) genome provides insights into vertebrate evolution. Nat Genet, 2013. 45 (4): p. 415-21, 421e1-2). Both proteins retain binding interfaces for cytoskeletal actin and the membrane-spanning glycoproteins of the Dystrophin (/Utrophin) Associated Protein Complex (D/UAPC). It is well established that full length recombinant derivatives of utrophin can reverse even severe muscular dystrophy phenotypes in mice (Tinsley, J., et al., Expression of full-length utrophin prevents muscular dystrophy in mdx mice. Nat Med, 1998. 4 (12): p. 1441-4; Gilbert, R., et al., Adenovirus-mediated utrophin gene transfer mitigates the dystrophic phenotype of mdx mouse muscles. Hum Gene Ther, 1999. 10 (8): p. 1299-310; and Odom, G. L., et al., Microutrophin delivery through rAAV6 increases lifespan and improves muscle function in dystrophic dystrophin/utrophin-deficient mice. Mol Ther, 2008. 16 (9): p. 1539-45). The unresolved gap in our knowledge has been the nature of the selective pressures that led to the initial tandem replication of the triple helical repeats of the ancestral protein, and whether the current physiological role(s) of dystrophin “requires” the estimated 6 nm/repeat×24 repeats or 144 nm length of the native protein Dp427. The severe clinical phenotype of some BMD patients with small internal deletions has suggested that Dp427's length is essential for its role as a shock absorber, but duplicational-BMD challenges this interpretation because these patients have longer than wild type dystrophins. An alternative interpretation emerges from our reconstruction of dystrophin's remote evolutionary history (Example 1).

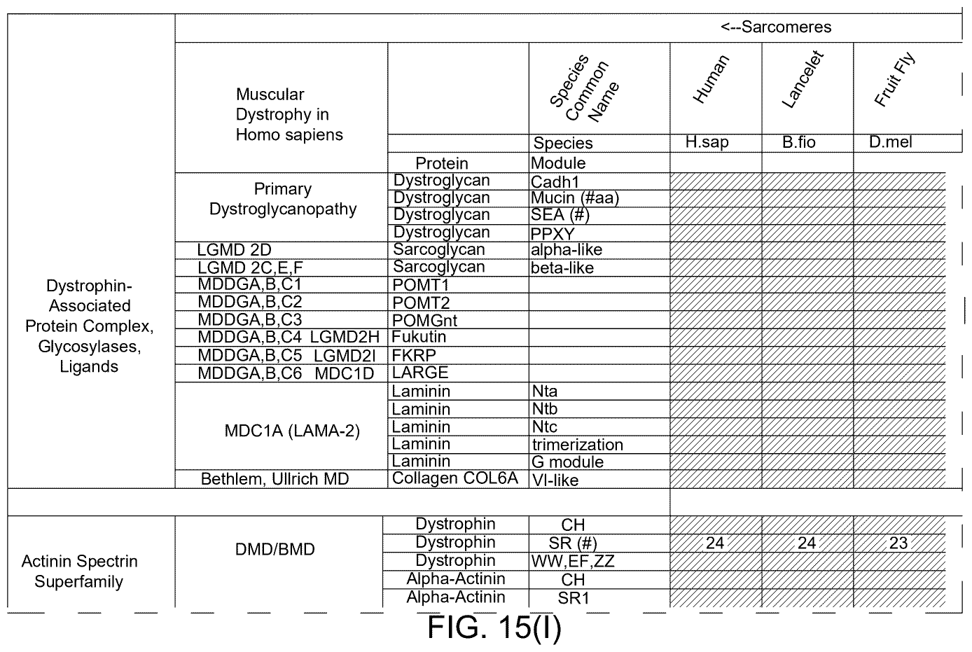

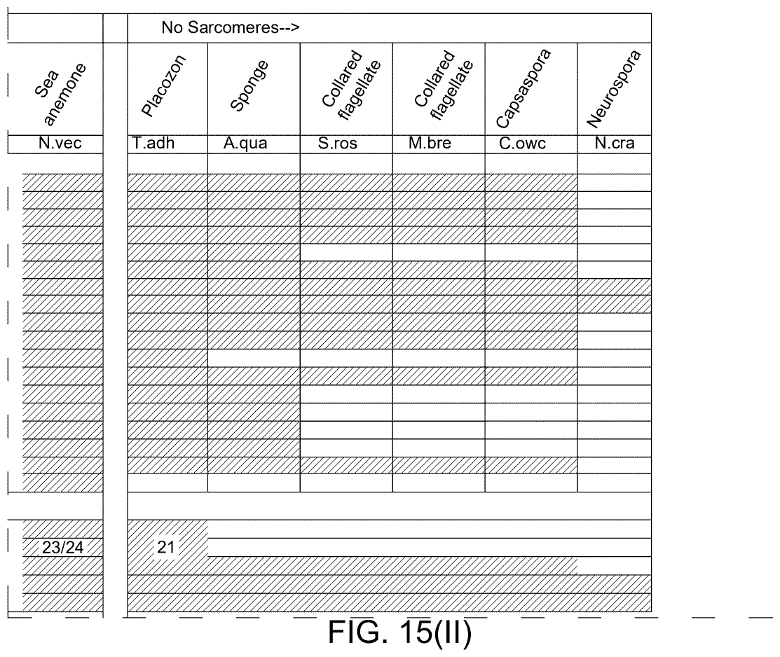

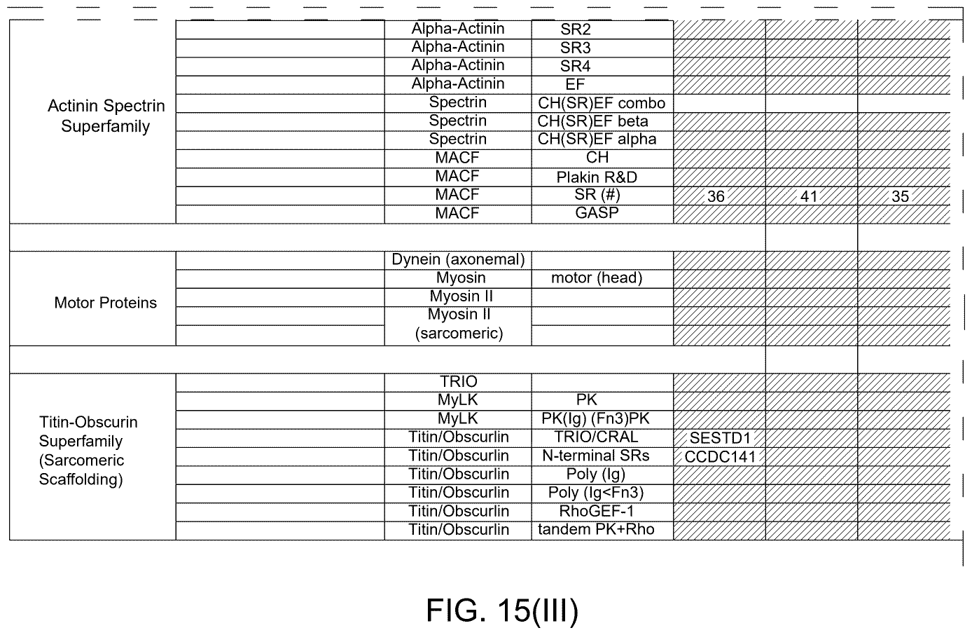

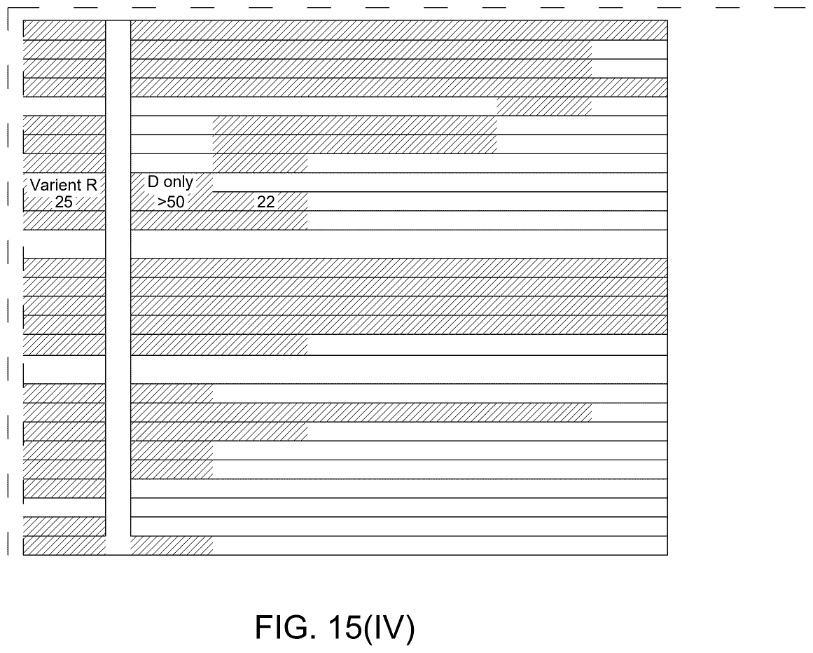

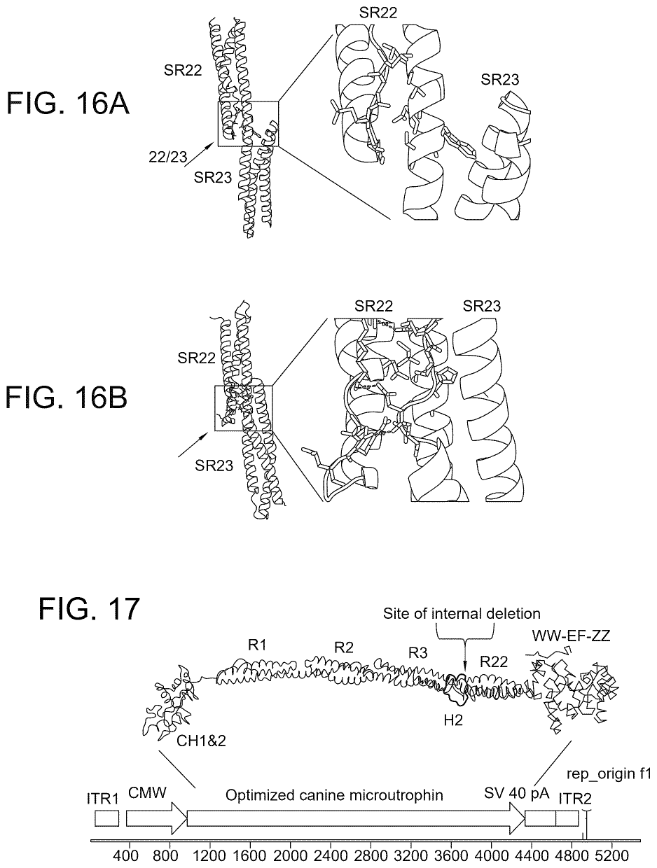

We asked the deceptively simple question “which came first, dystrophin or the sarcomere?” The goal was to ascertain whether available evidence from genomes and inferred proteomes of extant species was consistent with a model in which the length of the dystrophin rod increased during the period from the emergence of sarcomeres to the recent evolution of specialized fast-twitch muscles that are most susceptible to acute injury in dystrophin deficient mammals (Webster, C., et al., Fast muscle fibers are preferentially affected in Duchenne muscular dystrophy. Cell, 1988. 52 (4): p. 503-13; and Petrof, B. J., et al., Adaptations in myosin heavy chain expression and contractile function in dystrophic mouse diaphragm. Am. J. Physiol., 1993. 265: p. C834-C841). As shown in FIG. 15, the evidence supports a reconstruction in which dystrophin predated the sarcomere. Detailed analysis shows that dystrophin is identical in length in extant Vertebrate and Cnidarian (jellyfish) species, with highly conserved gene structures as shown in FIG. 6A to FIG. 6C. The length of dystrophin was likely achieved well before the emergence of sarcomeres, even before the divergence of Placozoa, a phylum represented by the simplest free-living animal species, Trichoplax adhaerens. This species uses ciliary dynein (not myosin) as its primary locomotive power source, and its body plan features only four cell types, none of which exhibit identifiable sarcomeres (Srivastava, M., et al., The Trichoplax genome and the nature of placozoans. Nature, 2008. 454 (7207): p. 955-60). An innovative analysis of gene structure provides compelling evidence that the rod-like domain of dystrophin, accounting for 80% of the protein's length, appears to have been co-opted in its entirety from a larger ancestral protein (not spectrin, note distinct patterns in FIG. 6B), with a distinct role in crosslinking actin filaments and microtubules (MACF). The essential implication of this finding is that an MACF-like protein, not dystrophin per se, was the subject of the selective pressure that drove the original lengthening of the two proteins' common ancestral rod domain. The crystal structure of MACF is distinct from that of spectrin with regard to the potential for longitudinal force transmission, providing an explanation for the preservation of the length of dystrophin and utrophin in a wide range of taxa. As shown in FIGS. 16A and 16B, spectrins and dystroplakins have triple helices that fold distinctly with regard to the extent of overlap and the number of stabilizing amino acid side chain interactions. In FIG. 16A and FIG. 16B, we model adjacent triple helical repeats of human utrophin using templates derived from (FIG. 16A) human beta2-spectrin (3EDV) and (FIG. 16B) human plectin (5J1G). Our model predicts that force is transmitted longitudinally across the broad interdomain interface in vivo, as required to transmit mechanical power from cell interior to extracellular matrix without disruption of the sarcolemma. Any major disruption in the side chain bonding between adjacent and potentially overlapping triple helical repeats would therefore destabilize dystrophin and utrophin during force transmission.

This model is entirely consistent with the proposition that the most important feature of the rod domains is strength and not length, with the extraordinary size of the proteins attributable to their historical legacy as derivatives by partial gene duplication of a much longer MACF homolog. Thus, short recombinant proteins specifically designed to optimize the structural integrity of the rod domain at all inter-repeat interfaces should fully complement the mechanical function of the full-length proteins, e.g. Dp427. The conceptually simplest way to approach this is to avoid internal rearrangements that directly juxtapose incompatible triple helical repeats. The problem is that crystallographic information on structure exists for only one of dystrophin's 24 triple helical repeats, and the inter-repeat primary structural homolog is low except for the conserved tryptophan residues in the center of helices A and C. We chose to initially focus on a recombinant protein with one internal and one C-terminal deletion relative to full length utrophin, and named it micro- or μ-Utrophin. As depicted in FIG. 17, this construct juxtaposes an unstructured, proline-rich inter-helical “hinge-2” domain against the last triple helical repeat, number 22 of full length Utrophin. In a best case scenario, the “hinge” could serve as an inter-helical spacer with the capacity to transmit longitudinal force without precise matching to the triple helical repeat 22 sequence. This was our rationale for a focused evaluation of μ-Utrophin, as described in detail below and in Example 2.

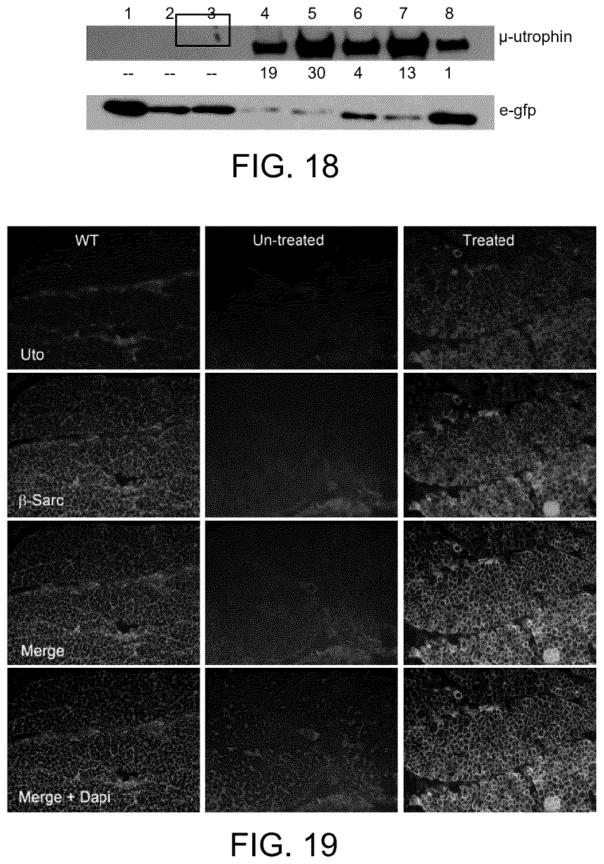

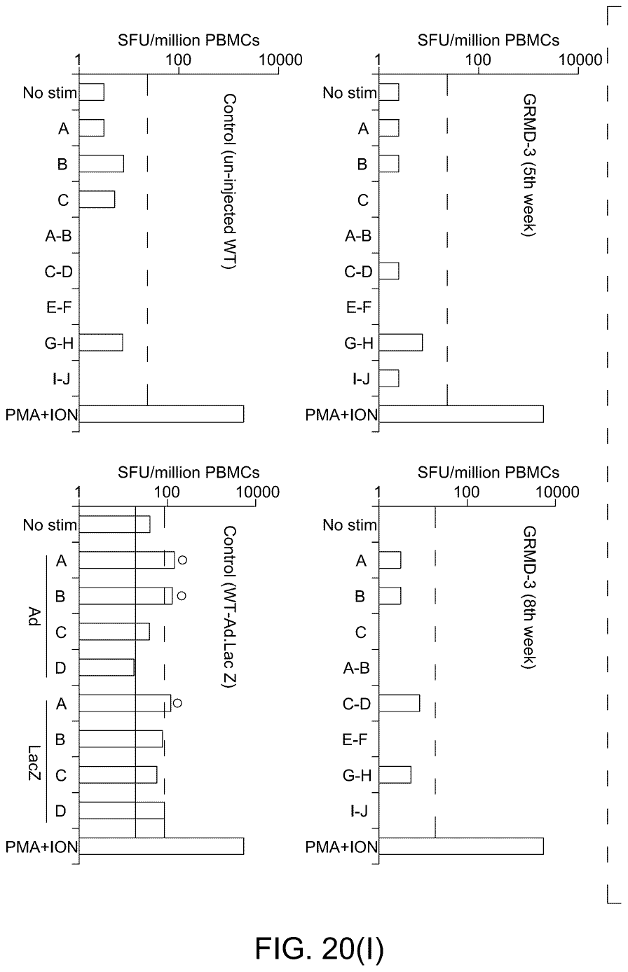

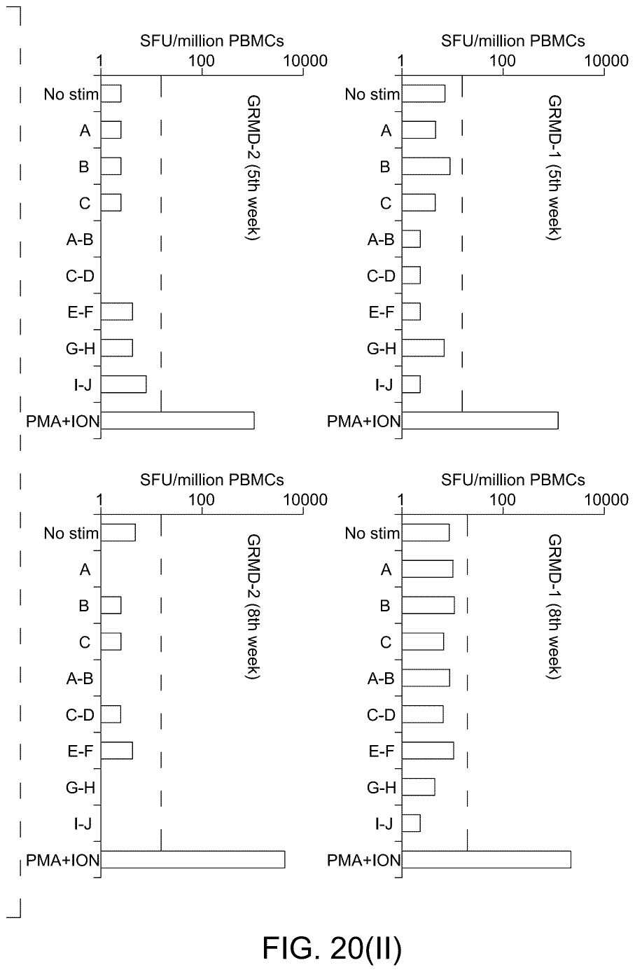

A systematic approach to μ-Utrophin transgene optimization determined that use of the codon bias for sarcomeric myosin heavy chain increased immunodetectable protein expression by 30-fold over wild type in vitro (lane 5 versus lane 8, FIG. 18). In fact the level of expression was so high that it competed effectively with expression of the cotransfected e-gfp control. To analyze the therapeutic efficacy of the optimized uU construct after packaging into AAV9, we performed a series of randomized, blinded investigations in the mdx mouse model of DMD, as described in Example 2.

On the basis of in silico analysis of MHC binding of minimal T cell peptide epitopes, the μUtrophin has fewer than 100th the number of potentially immunodominant foreign peptides compared with all hypothetical μDystrophins in the dystrophin-deficient host. This minimizes the risk of post-therapy autoimmunity in DMD, and the potential requirement for chronic immunosuppression. Our blinded studies of systemic μUtrophin expression in non-immunosuppressed GRMD dogs provided further reassurance by documenting the complete absence of peripheral T cell reactivity by interferon gamma ELISpot assay. These studies were necessarily performed with a dose (normalized to adult body weight) 1/10th of the maximal dose used in mdx mice, and could only be extended at a future date to assess the maximal tolerated dose in the large animal DMD model with the scalable production technology.

### F. Mechanobiology of Dystrophin Rationale and Structure-Nano-Utrophin

Our findings have important implications for the entire field, as it pertains equally to all AAV-sized dystrophin candidates that are being developed for translation into clinical studies.

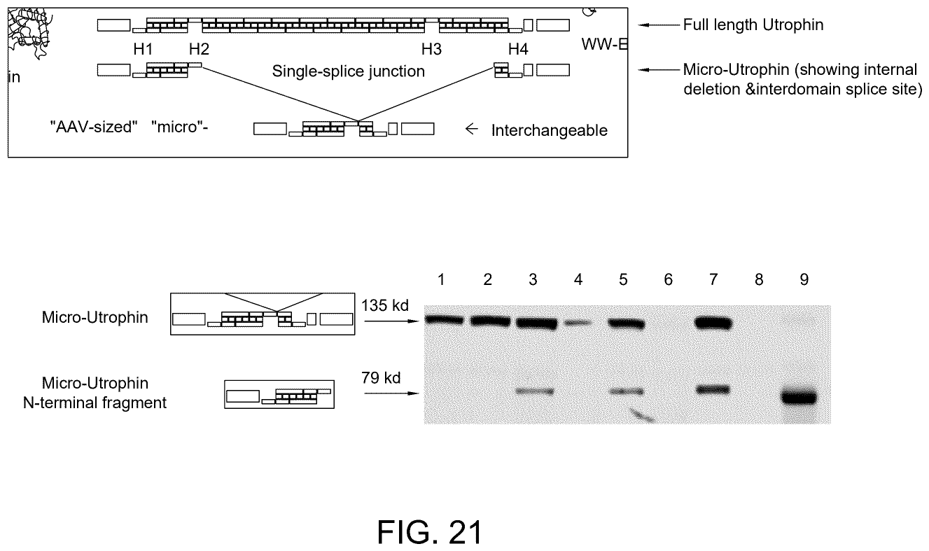

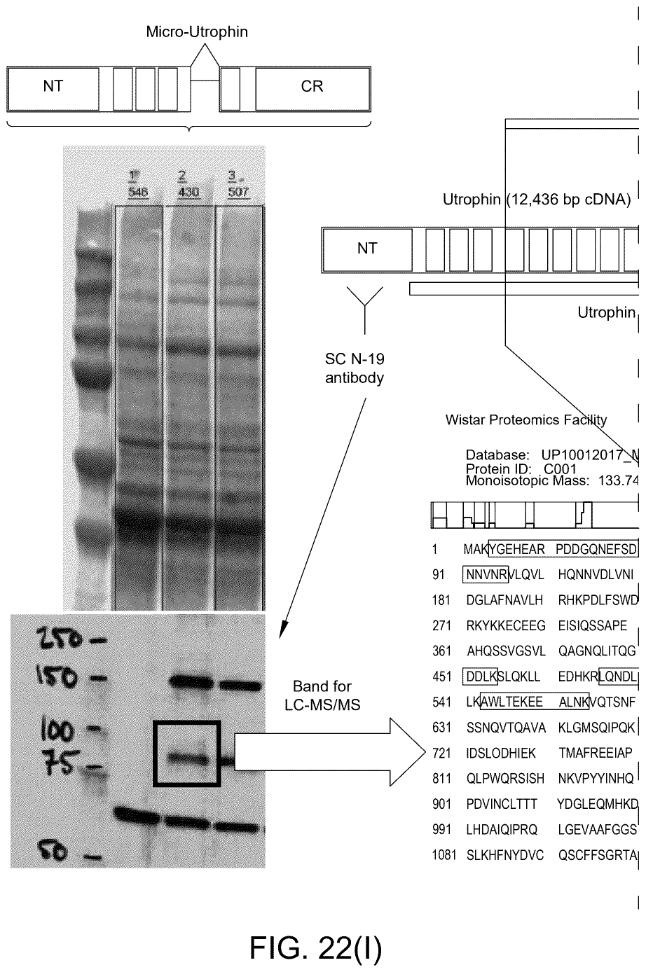

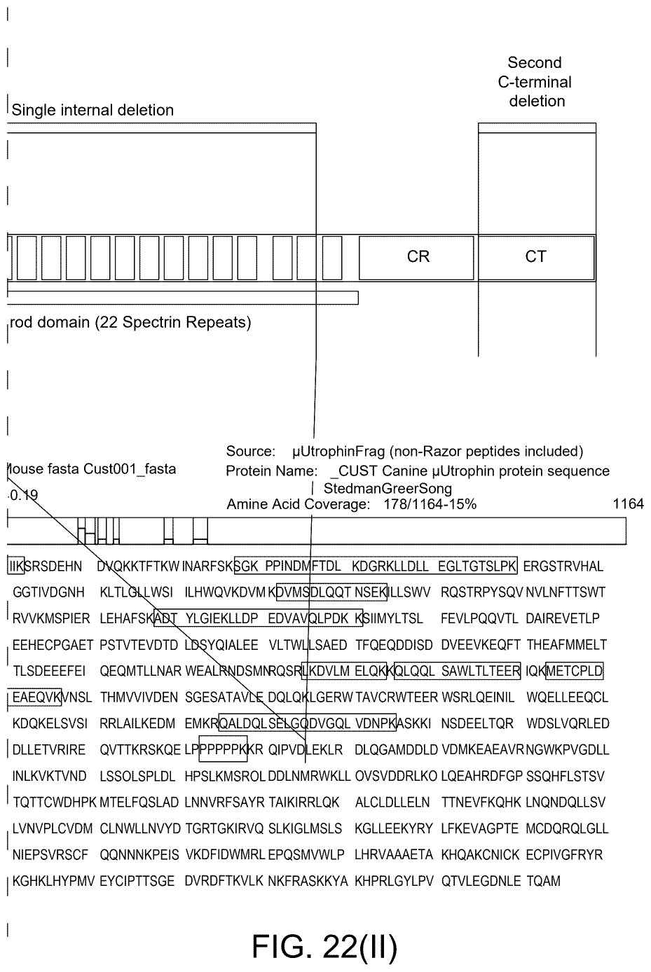

We initiated a series of blinded experiments to address the stability of μ-Utrophin in AAV-injected mdx mice. These quantitative studies have already provided very favorable data at intermediate timepoints with regard to the levels of μ-utrophin expression. However, in further analysis we noted an unanticipated “extra band” on western blots after staining with an antibody specific to the N-terminus of utrophin. As summarized in FIG. 21, the molecular weight assigned to this crisp band exactly matches that of the 79 kd N-terminal portion of μ-Utrophin. In other words, the findings suggest that the 135 kd μ-Utrophin was disrupted in immediate proximity to the junction between the portion from “Hinge 2” and spectrin-like repeat 22, flanking the coding sequence deletion corresponding to full length utrophin, thereby releasing as a fragment the 79 kd N-terminal “subdomain”. In our studies there appears to be a correlation between the appearance and intensity of the 79 kd band and the maximal recent level of force transduction by the muscle. We next subjected the 79 kd region of additional gels to proteomic analysis by the combination of liquid chromatography and tandem mass spectroscopy. This confirmed our hypothesis, as shown in FIG. 22. This finding compelled us to reconsider our assumption about the interchangeability of spectrin repeats, specifically 4 and 22 when placed in immediate primary structural proximity to hinge 2. In revisiting the findings of our bioinformatic analysis of dystrophin, utrophin, and titin, it became clear that the poly IgG domains of titin show strong evidence for interchangeability over evolutionary time, but that in identical species comparisons we see no evidence for interchangeability in the other two proteins (Example 1). This provides a compelling explanation for the genotype-phenotype correlation in selected cases of Becker MD, especially severe cases associated with gene duplications in which by inference there exists a longer than wild type dystrophin that has a weakest link for axial force transmission across the novel junction of two ancestrally non-adjacent portions of the rod.

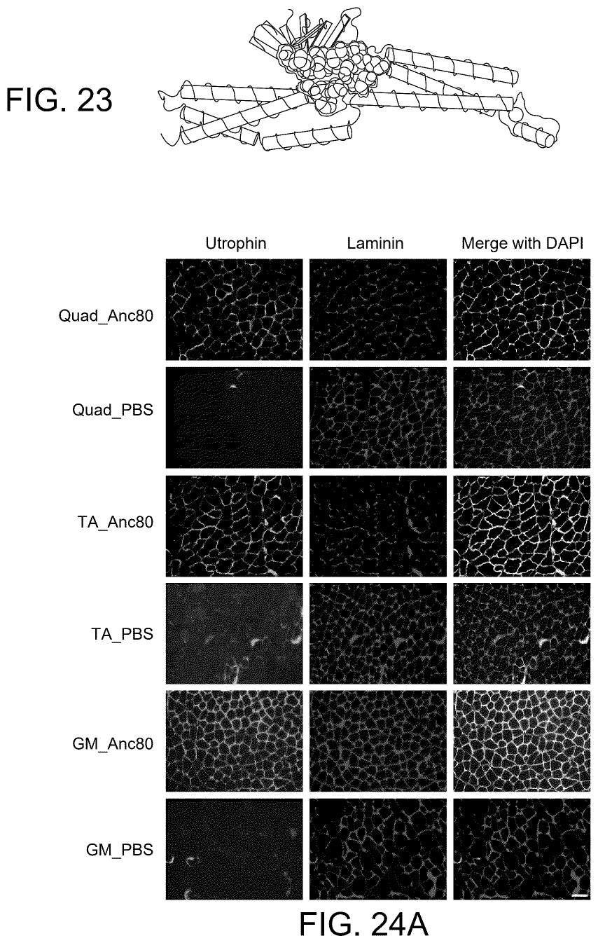

One crystal structure from the MACF/plakin protein family is potentially informative, as structural data for dystrophin and utrophin are limited to the first triple helical repeat (i.e. extreme N-terminal end of the rod), and the hinge domains are predicted to be “unstructured”. Prior to the determination of this structure there was speculation that the SH3 domain between triple helical repeats also served as a hinge, conjuring images of a wobbly interface between two strong portions of a rod. The structure reveals, in contrast, that the SH3 domain makes multiple high affinity contacts with compatible amino acid side chains from the adjacent triple helices on both sides, a configuration with the potential to transmit longitudinal force and resist unfolding (FIG. 23 showing 3PEo structure SR4 & 5 of plakin domain, SH3: SR4/5 binding interface in spacefill). Analysis of the primary structures of several dystrophin/utrophin orthologs from species of Cnidaria (jellyfish) shows the presence of intercalated but HMM recognizable domains but no unstructured “hinges”, further supporting our hypotheses about both the evolutionary origin of the dystrophin rod domain from an MACF-like protein and the magnitude of long-axis force transmission across the broad surface area of the inter-domain interfaces.

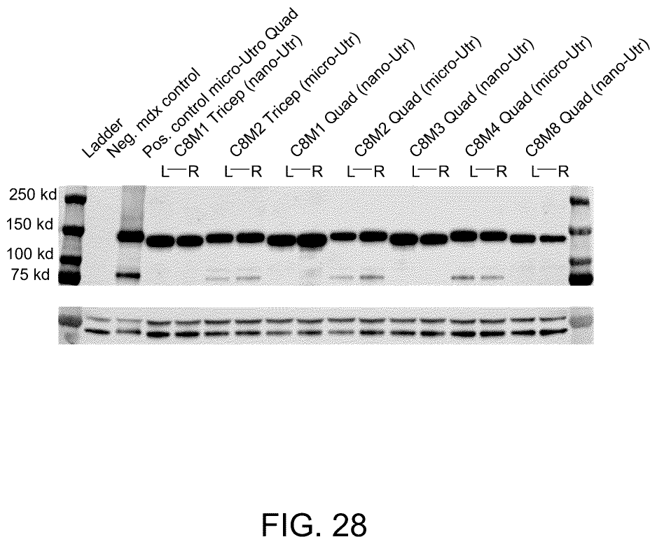

With these structural and functional constraints in mind, we revisited the design of AAV-compatible, miniaturized substitutes for Dp427, i.e. full length dystrophin. We designed a nano-Utrophin that takes advantage of a unique opportunity to internally rearrange the rod domain with the least disruption to the interdomain interfaces. This is accomplished in silico by merging pairs of disparate triple helices across a conserved axial cross section, the one illustrated at the site of the interacting, highly-conserved tryptophan residues stabilizing the core of the triple helix (FIG. 3). We have synthesized codon-optimized cDNAs for this construct, created and maxi-prepped the ITR-flanked transcriptional cassette vectors for triple transfection of 293 cells. Further, we conducted a blinded experiment in which mdx mice were randomized to receive AAVs encoding either “micro-” or “nano-” utrophin. The results showed that micro-utropin was cleaved precisely at the end of the 79 kd N-terminal subfragment, while there was no detectable cleavage of the nano-utrophin under similar physiological loading (FIG. 28). Both proteins were properly localized to the sarcolemma in muscles of mdx mice. The findings from this experiment indicate that the superior strength of nano-utrophin compared to micro-utrophin more closely approximates the mechanobiology of dystrophin isoform Dp427. Additional studies are performed utilizing the methods used above in these Examples to evaluate phenotypic amelioration in various disease models. In parallel, the extent of any improvement in the efficiency of transgene packaging into AAV is established on the basis of reduced vector genome size.

### G. Reconstructed Ancestral AAV Capsids for DMD Therapeutics: Anc80, 81, 82

AAV vectors based on the naturally occurring capsid serotype 9 achieve spectacular global biodistribution to striated muscle in dogs and non-human primates. The structural basis for this is poorly understood, although the capsid residues involved in binding to selected membrane receptors are well defined for other serotypes. AAV8 is a better choice for efficient cardiac gene transfer in dogs, reflecting a species difference since AAV9 provides robust transduction of the myocardium in primates. Both AAV8 and 9 are associated with neutralizing antibodies in significant proportions of the adult human population. In an effort to circumvent this limitation while maintaining global biodistribution to striated muscle, Zinn, et al (Zinn, E., et al., In Silico Reconstruction of the Viral Evolutionary Lineage Yields a Potent Gene Therapy Vector. Cell Rep, 2015. 12 (6): p. 1056-68), used the combination of ancestral sequence reconstruction in silico and gene synthesis in vitro to prepare AAVs with “novel” vector capsids (i.e. likely recapitulating naturally occurring but long ago extinct capsid variants). Among these, the ones labeled Anc80, 81, and 82 represent the most immediate opportunities to replicate or extend the favorable biodistribution of AAV8 and 9 while expanding the eligibility pool from patient populations with the potential for high titer neutralizing antibodies after historical exposure to naturally occurring extant AAVs, as described in detail (Zinn et al, as cited above).

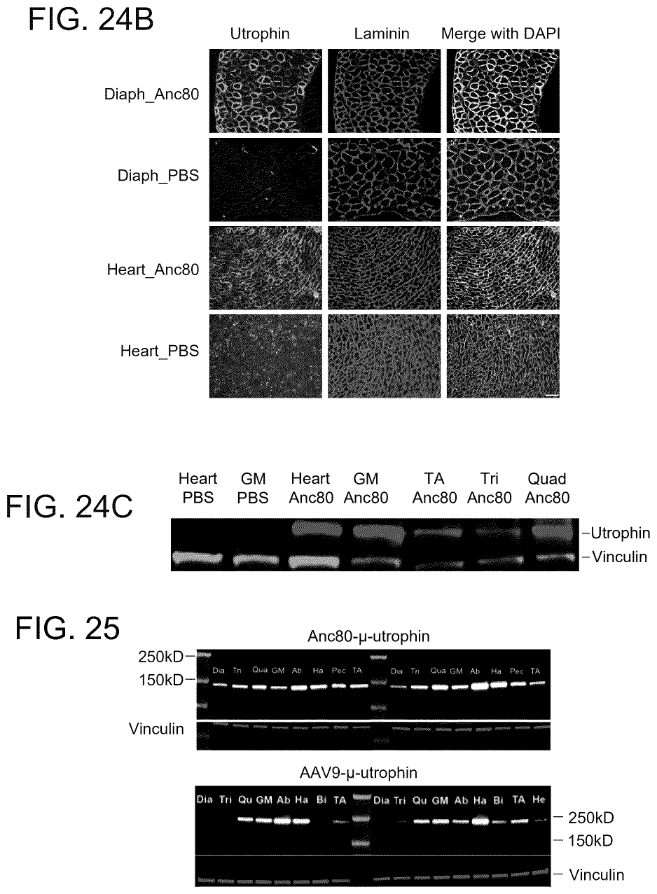

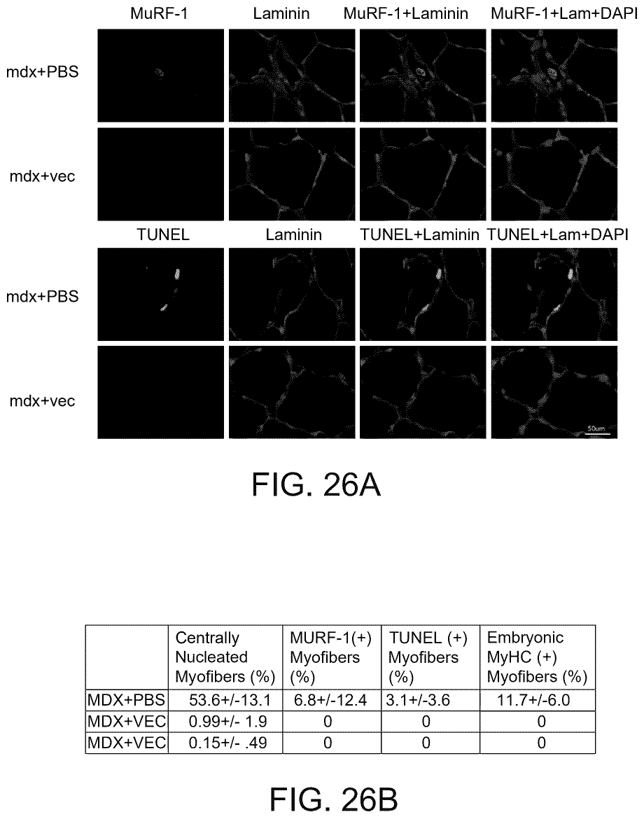

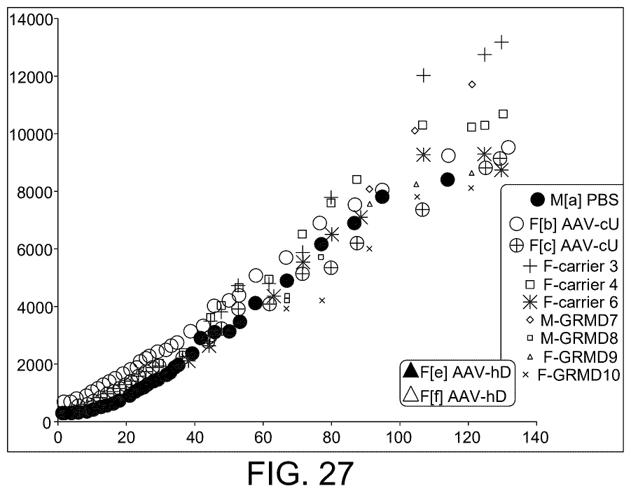

We recently packaged our μUtrophin genome in the Anc80 capsid and used the resulting vector to evaluate transduction of relevant muscles in mdx mice. These studies show that Anc80 achieves a global biodistribution comparable to that of AAV9 in this context, with strong transduction of cardiac and skeletal muscle (FIG. 24A to 24C).

Data from randomized, blinded experiments confirm that Anc80 and AAV9 have the capacity for comparable infectivity in vivo in dystrophic muscle, based on the levels of expression of a therapeutic transgene.

Experiments designed to qualitatively compare AAV9 and Anc80 were performed for biodistribution of μUtrophin in cohorts of mdx mice following systemic administration of these vectors in equal doses of 2.5×10¹² vg/mouse. Representative western blots from multiple muscles from two mice for each vector are shown in FIG. 25, demonstrating widespread and efficient transduction of striated muscles with both vectors.

### Example 4—Five-Repeat Mutant Utrophin and Dystrophin

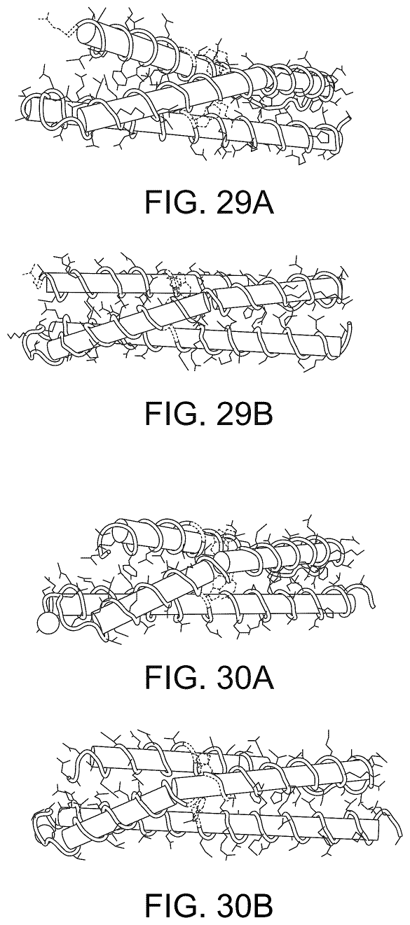

We have designed utrophin and dystrophin recombinant proteins which contain an additional triple helix relative to the original “nano” mutants described above. These “five-repeat” mutants have 5 spectrin-like triple-helical repeats between the N-terminal calponin homology domains and the C-terminal “WW-EF-ZZ” domains present in the full-length proteins. These variants also have improved stability due to the presence of a triple splice mutation and, further, are capable of being packaged into AAV vectors. Importantly, the development of these mutants illustrates that the principles relied on to design four-repeat nano-dystrophins and nano-utrophins, including those described herein, can be extended to variants having five helical repeats. An amino acid sequence corresponding to a five-repeat dystophin is provided in SEQ ID NO: 22, wherein a splice mutation was formed by joining helical repeats 1 and 20 of the full-length dystophin protein (FIG. 29A and FIG. 29B). An amino acid sequence corresponding to a five-repeat utrophin is provided in SEQ ID NO: 21, wherein a splice mutation was formed by joining helical repeats 1 and 18 of the full-length utrophin protein (FIG. 30A and FIG. 30B).

All publications, patents, patent applications, cited in this application and the Sequence Listing referenced herein, as well as U.S. Provisional Patent Application No. 62/658,464, filed Apr. 16, 2018, are hereby incorporated by reference in their entireties as if each individual publication or patent application were specifically and individually indicated to be incorporated by reference. Numerous modifications and variations are included in the scope of the above-identified specification and are expected to be obvious to one of skill in the art. Such modifications and alterations to the compositions and processes, such as selections of different coding sequences or selection or dosage of the vectors or immune modulators are believed to be within the scope of the claims appended hereto.

1. A recombinant adeno-associated virus (rAAV) having an AAV capsid and a vector genome, wherein the vector genome comprises a nucleic acid sequence encoding a dystrophin superfamily mutant protein comprising a hybrid triple helical domain under control of regulatory sequences which direct expression thereof, the hybrid triple helical domain comprising
a first helix comprising an N-terminal portion of a helix A fused to a C-terminal portion of a helix A′;
a second helix comprising an N-terminal portion of a helix B′ fused to a C-terminal portion of a helix B; and
a third helix comprising an N-terminal portion of a helix C fused to a C-terminal portion of helix C′;
wherein helices A, B, and C are present in a first triple helical repeat that is non-adjacent to a second triple helical repeat having helices A′, B′, and C′ in a native dystrophin superfamily protein.

2. The rAAV according to claim 1, wherein the triple splice mutant protein is a triple splice mutant dystrophin and comprises a deletion in:
(a) at least helical repeat 3 to helical repeat 21 of the full-length dystrophin;
(b) at least helical repeat 3 to helical repeat 23 of the full-length dystrophin; or
(c) at least helical repeat 2 to helical repeat 19 of the full-length dystrophin.

3. The rAAV according to claim 1 wherein the triple splice mutant protein comprises the amino acid sequence of: SEQ ID NO: 1.

4. The rAAV according to claim 1, wherein the triple splice mutant protein comprises the amino acid sequence of SEQ ID NO: 22.

5. The rAAV according to claim 1, wherein the dystrophin superfamily mutant protein is a triple splice mutant utrophin and comprises a deletion in:
(a) at least helical repeat 3 to helical repeat 19 of full-length utrophin; or
(b) at least helical repeat 2 to helical repeat 17 of full-length utrophin.

6. The rAAV according to claim 5, wherein the triple mutant protein comprises the amino acid sequence of SEQ ID NO: 3.

7. The rAAV according to claim 1, wherein the triple mutant protein comprises the amino acid sequence of SEQ ID NO: 20.

8. The rAAV according to claim 7, wherein the nucleic acid sequence encoding the triple splice mutant protein comprises SEQ ID NO: 19, or a sequence about 95% to about 99% identical to SEQ ID NO: 19.

9. The rAAV according to claim 5, wherein the triple mutant protein comprises the amino acid sequence of SEQ ID NO: 21.

10. The rAAV according to claim 1, wherein the recombinant adeno-associated virus is selected from AAV1, AAV5, AAV6, AAV8, AAV8 triple, AAV9, Anc80, Anc81 and Anc82.

11. A recombinant dystrophin superfamily triple splice mutant protein comprising a hybrid triple helix domain comprising
a first helix comprising an N-terminal portion of a helix A fused to a C-terminal portion of a helix A′;
a second helix comprising an N-terminal portion of a helix B′ fused to a C-terminal portion of a helix B; and
a third helix comprising an N-terminal portion of a helix C fused to a C-terminal portion of helix C′;
wherein helices A, B, and C are present in a first triple helical repeat that is non-adjacent to a second triple helical repeat having helices A′, B′, and C′ in a native dystrophin superfamily protein.

12. The recombinant protein according to claim 11, wherein the dystrophin superfamily mutant protein is a triple splice mutant dystrophin and comprises a deletion in:
(a) at least helical repeat 3 to helical repeat 21 of the full-length dystrophin;
(b) at least helical repeat 3 to helical repeat 23 of the full-length dystrophin;
(c) at least helical repeat 2 to helical repeat 19 of the full-length dystrophin.

13. The recombinant protein according to claim 11, wherein the dystrophin superfamily mutant protein is a triple splice mutant utrophin and comprises a deletion in:
(a) at least helical repeat 3 to helical repeat 19 of full-length utrophin; or
(b) in at least helical repeat 2 to helical repeat 17 of full-length utrophin.

14. The recombinant protein according to claim 13, wherein the triple mutant protein comprises the amino acid sequence of SEQ ID NO: 3.

15. The recombinant protein according to claim 11, wherein the triple mutant protein comprises the amino acid sequence of SEQ ID NO: 20.

16. The recombinant protein according to claim 15, wherein the nucleic acid sequence encoding the triple splice mutant protein comprises SEQ ID NO: 19, or a sequence about 95% to about 99% identical to SEQ ID NO: 19.

17. The recombinant protein according to claim 11, wherein the triple mutant protein comprises the amino acid sequence of SEQ ID NO: 21.

18. A recombinant nucleic acid sequence encoding a protein according to claim 11.

19. A pharmaceutical composition comprising a rAAV according to claim 1 in a formulation buffer.

20. A method of treating a subject diagnosed with Duchenne muscular dystrophy comprising administering to the subject the composition of claim 19.


SEQUENCE LISTING (25 sequences)

SEQ ID NO: 1 - protein, 1131 aa, synthetic construct
  REGION 1..1131: note = amino acid sequence of nano-dystrophin
  REGION 1..463: note = MISC_FEATURE - amino acid sequence identical to N-terminal region of full-length human dystrophin.
  SITE 463: note = MISC_FEATURE - tryptophan residue in the "A" helix at the core of the Hidden Markov Model (HMM) and all crystal structures for proteins in this superfamily.
  REGION 464..505: note = MISC_FEATURE - amino acid sequence identical to C-terminal region of full-length human dystrophin.
  REGION 505..506: note = MISC_FEATURE - positions within the superfamily HMM for the "B" helix that flank the hypothetical plane of transection as depicted in FIG 2F
  REGION 506..550: note = MISC_FEATURE - amino acid sequence identical to N-terminal region of full-length human dystrophin.
  SITE 550: note = MISC_FEATURE - tryptophan residue in the "C" helix at the core of the Hidden Markov Model (HMM) and all crystal structures for proteins in this superfamily.
  REGION 551..1131: note = MISC_FEATURE - amino acid sequence identical to C-terminal region of full-length human dystrophin.
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGEIEQEQVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 2 - DNA, 3393 bp, synthetic construct
  misc_feature 1..3393: note = a nucleic acid sequence encoding nano-dystrophin
  misc_feature 6: note = n is a, c, g, or t
  misc_feature 21: note = n is a, c, g, or t
  misc_feature 39: note = n is a, c, g, or t
  misc_feature 48: note = n is a, c, g, or t
  misc_feature 60: note = n is a, c, g, or t
  misc_feature 66: note = n is a, c, g, or t
  misc_feature 75: note = n is a, c, g, or t
  misc_feature 81: note = n is a, c, g, or t
  misc_feature 90: note = n is a, c, g, or t
  misc_feature 99: note = n is a, c, g, or t
  misc_feature 120: note = n is a, c, g, or t
  misc_feature 126: note = n is a, c, g, or t
  misc_feature 132: note = n is a, c, g, or t
  misc_feature 141: note = n is a, c, g, or t
  misc_feature 144: note = n is a, c, g, or t
  misc_feature 147: note = n is a, c, g, or t
  misc_feature 150: note = n is a, c, g, or t
  misc_feature 153: note = n is a, c, g, or t
  misc_feature 159: note = n is a, c, g, or t
  misc_feature 162: note = n is a, c, g, or t
  misc_feature 168: note = n is a, c, g, or t
  misc_feature 171: note = n is a, c, g, or t
  misc_feature 174: note = n is a, c, g, or t
  misc_feature 177: note = n is a, c, g, or t
  misc_feature 186: note = n is a, c, g, or t
  misc_feature 189: note = n is a, c, g, or t
  misc_feature 201: note = n is a, c, g, or t
  misc_feature 204: note = n is a, c, g, or t
  misc_feature 207: note = n is a, c, g, or t
  misc_feature 210: note = n is a, c, g, or t
  misc_feature 213: note = n is a, c, g, or t
  misc_feature 219: note = n is a, c, g, or t
  misc_feature 222: note = n is a, c, g, or t
  misc_feature 231: note = n is a, c, g, or t
  misc_feature 240: note = n is a, c, g, or t
  misc_feature 243: note = n is a, c, g, or t
  misc_feature 246: note = n is a, c, g, or t
  misc_feature 249: note = n is a, c, g, or t
  misc_feature 252: note = n is a, c, g, or t
  misc_feature 267: note = n is a, c, g, or t
  misc_feature 273: note = n is a, c, g, or t
  misc_feature 276: note = n is a, c, g, or t
  misc_feature 285: note = n is a, c, g, or t
  misc_feature 288: note = n is a, c, g, or t
  misc_feature 291: note = n is a, c, g, or t
  misc_feature 300: note = n is a, c, g, or t
  misc_feature 306: note = n is a, c, g, or t
  misc_feature 318: note = n is a, c, g, or t
  misc_feature 321: note = n is a, c, g, or t
  misc_feature 324: note = n is a, c, g, or t
  misc_feature 327: note = n is a, c, g, or t
  misc_feature 330: note = n is a, c, g, or t
  misc_feature 348: note = n is a, c, g, or t
  misc_feature 360: note = n is a, c, g, or t
  misc_feature 369: note = n is a, c, g, or t
  misc_feature 387: note = n is a, c, g, or t
  misc_feature 390: note = n is a, c, g, or t
  misc_feature 393: note = n is a, c, g, or t
  misc_feature 402: note = n is a, c, g, or t
  misc_feature 408: note = n is a, c, g, or t
  misc_feature 420: note = n is a, c, g, or t
  misc_feature 423: note = n is a, c, g, or t
  misc_feature 426: note = n is a, c, g, or t
  misc_feature 432: note = n is a, c, g, or t
  misc_feature 435: note = n is a, c, g, or t
  misc_feature 441: note = n is a, c, g, or t
  misc_feature 444: note = n is a, c, g, or t
  misc_feature 447: note = n is a, c, g, or t
  misc_feature 456: note = n is a, c, g, or t
  misc_feature 462: note = n is a, c, g, or t
  misc_feature 468: note = n is a, c, g, or t
  misc_feature 480: note = n is a, c, g, or t
  misc_feature 483: note = n is a, c, g, or t
  misc_feature 486: note = n is a, c, g, or t
  misc_feature 492: note = n is a, c, g, or t
  misc_feature 498: note = n is a, c, g, or t
  misc_feature 501: note = n is a, c, g, or t
  misc_feature 504: note = n is a, c, g, or t
  misc_feature 507: note = n is a, c, g, or t
  misc_feature 513: note = n is a, c, g, or t
  misc_feature 516: note = n is a, c, g, or t
  misc_feature 525: note = n is a, c, g, or t
  misc_feature 531: note = n is a, c, g, or t
  misc_feature 534: note = n is a, c, g, or t
  misc_feature 540: note = n is a, c, g, or t
  misc_feature 555: note = n is a, c, g, or t
  misc_feature 558: note = n is a, c, g, or t
  misc_feature 561: note = n is a, c, g, or t
  misc_feature 573: note = n is a, c, g, or t
  misc_feature 576: note = n is a, c, g, or t
  misc_feature 579: note = n is a, c, g, or t
  misc_feature 585: note = n is a, c, g, or t
  misc_feature 588: note = n is a, c, g, or t
  misc_feature 597: note = n is a, c, g, or t
  misc_feature 609: note = n is a, c, g, or t
  misc_feature 612: note = n is a, c, g, or t
  misc_feature 621: note = n is a, c, g, or t
  misc_feature 624: note = n is a, c, g, or t
  misc_feature 636: note = n is a, c, g, or t
  misc_feature 639: note = n is a, c, g, or t
  misc_feature 645: note = n is a, c, g, or t
  misc_feature 654: note = n is a, c, g, or t
  misc_feature 660: note = n is a, c, g, or t
  misc_feature 663: note = n is a, c, g, or t
  misc_feature 669: note = n is a, c, g, or t
  misc_feature 681: note = n is a, c, g, or t
  misc_feature 687: note = n is a, c, g, or t
  misc_feature 699: note = n is a, c, g, or t
  misc_feature 702: note = n is a, c, g, or t
  misc_feature 705: note = n is a, c, g, or t
  misc_feature 714: note = n is a, c, g, or t
  misc_feature 717: note = n is a, c, g, or t
  misc_feature 720: note = n is a, c, g, or t
  misc_feature 729: note = n is a, c, g, or t
  misc_feature 732: note = n is a, c, g, or t
  misc_feature 741: note = n is a, c, g, or t
  misc_feature 753: note = n is a, c, g, or t
  misc_feature 762: note = n is a, c, g, or t
  misc_feature 765: note = n is a, c, g, or t
  misc_feature 768: note = n is a, c, g, or t
  misc_feature 771: note = n is a, c, g, or t
  misc_feature 774: note = n is a, c, g, or t
  misc_feature 780: note = n is a, c, g, or t
  misc_feature 783: note = n is a, c, g, or t
  misc_feature 804: note = n is a, c, g, or t
  misc_feature 825: note = n is a, c, g, or t
  misc_feature 837: note = n is a, c, g, or t
  misc_feature 840: note = n is a, c, g, or t
  misc_feature 843: note = n is a, c, g, or t
  misc_feature 846: note = n is a, c, g, or t
  misc_feature 849: note = n is a, c, g, or t
  misc_feature 855: note = n is a, c, g, or t
  misc_feature 864: note = n is a, c, g, or t
  misc_feature 867: note = n is a, c, g, or t
  misc_feature 870: note = n is a, c, g, or t
  misc_feature 873: note = n is a, c, g, or t
  misc_feature 876: note = n is a, c, g, or t
  misc_feature 882: note = n is a, c, g, or t
  misc_feature 885: note = n is a, c, g, or t
  misc_feature 894: note = n is a, c, g, or t
  misc_feature 900: note = n is a, c, g, or t
  misc_feature 906: note = n is a, c, g, or t
  misc_feature 912: note = n is a, c, g, or t
  misc_feature 915: note = n is a, c, g, or t
  misc_feature 921: note = n is a, c, g, or t
  misc_feature 924: note = n is a, c, g, or t
  misc_feature 927: note = n is a, c, g, or t
  misc_feature 930: note = n is a, c, g, or t
  misc_feature 936: note = n is a, c, g, or t
  misc_feature 939: note = n is a, c, g, or t
  misc_feature 942: note = n is a, c, g, or t
  misc_feature 945: note = n is a, c, g, or t
  misc_feature 948: note = n is a, c, g, or t
  misc_feature 954: note = n is a, c, g, or t
  misc_feature 957: note = n is a, c, g, or t
  misc_feature 966: note = n is a, c, g, or t
  misc_feature 972: note = n is a, c, g, or t
  misc_feature 975: note = n is a, c, g, or t
  misc_feature 987: note = n is a, c, g, or t
  misc_feature 993: note = n is a, c, g, or t
  misc_feature 996: note = n is a, c, g, or t
  misc_feature 999: note = n is a, c, g, or t
  misc_feature 1002: note = n is a, c, g, or t
  misc_feature 1011: note = n is a, c, g, or t
  misc_feature 1017: note = n is a, c, g, or t
  misc_feature 1023: note = n is a, c, g, or t
  misc_feature 1029: note = n is a, c, g, or t
  misc_feature 1038: note = n is a, c, g, or t
  misc_feature 1041: note = n is a, c, g, or t
  misc_feature 1044: note = n is a, c, g, or t
  misc_feature 1053: note = n is a, c, g, or t
  misc_feature 1056: note = n is a, c, g, or t
  misc_feature 1059: note = n is a, c, g, or t
  misc_feature 1065: note = n is a, c, g, or t
  misc_feature 1068: note = n is a, c, g, or t
  misc_feature 1071: note = n is a, c, g, or t
  misc_feature 1074: note = n is a, c, g, or t
  misc_feature 1083: note = n is a, c, g, or t
  misc_feature 1086: note = n is a, c, g, or t
  misc_feature 1092: note = n is a, c, g, or t
  misc_feature 1098: note = n is a, c, g, or t
  misc_feature 1107: note = n is a, c, g, or t
  misc_feature 1116: note = n is a, c, g, or t
  misc_feature 1122: note = n is a, c, g, or t
  misc_feature 1125: note = n is a, c, g, or t
  misc_feature 1143: note = n is a, c, g, or t
  misc_feature 1152: note = n is a, c, g, or t
  misc_feature 1167: note = n is a, c, g, or t
  misc_feature 1170: note = n is a, c, g, or t
  misc_feature 1173: note = n is a, c, g, or t
  misc_feature 1182: note = n is a, c, g, or t
  misc_feature 1185: note = n is a, c, g, or t
  misc_feature 1188: note = n is a, c, g, or t
  misc_feature 1191: note = n is a, c, g, or t
  misc_feature 1200: note = n is a, c, g, or t
  misc_feature 1206: note = n is a, c, g, or t
  misc_feature 1209: note = n is a, c, g, or t
  misc_feature 1212: note = n is a, c, g, or t
  misc_feature 1218: note = n is a, c, g, or t
  misc_feature 1224: note = n is a, c, g, or t
  misc_feature 1227: note = n is a, c, g, or t
  misc_feature 1230: note = n is a, c, g, or t
  misc_feature 1236: note = n is a, c, g, or t
  misc_feature 1239: note = n is a, c, g, or t
  misc_feature 1254: note = n is a, c, g, or t
  misc_feature 1260: note = n is a, c, g, or t
  misc_feature 1278: note = n is a, c, g, or t
  misc_feature 1281: note = n is a, c, g, or t
  misc_feature 1287: note = n is a, c, g, or t
  misc_feature 1290: note = n is a, c, g, or t
  misc_feature 1302: note = n is a, c, g, or t
  misc_feature 1305: note = n is a, c, g, or t
  misc_feature 1308: note = n is a, c, g, or t
  misc_feature 1311: note = n is a, c, g, or t
  misc_feature 1314: note = n is a, c, g, or t
  misc_feature 1329: note = n is a, c, g, or t
  misc_feature 1335: note = n is a, c, g, or t
  misc_feature 1341: note = n is a, c, g, or t
  misc_feature 1344: note = n is a, c, g, or t
  misc_feature 1347: note = n is a, c, g, or t
  misc_feature 1356: note = n is a, c, g, or t
  misc_feature 1371: note = n is a, c, g, or t
  misc_feature 1380: note = n is a, c, g, or t
  misc_feature 1392: note = n is a, c, g, or t
  misc_feature 1395: note = n is a, c, g, or t
  misc_feature 1401: note = n is a, c, g, or t
  misc_feature 1407: note = n is a, c, g, or t
  misc_feature 1410: note = n is a, c, g, or t
  misc_feature 1413: note = n is a, c, g, or t
  misc_feature 1419: note = n is a, c, g, or t
  misc_feature 1422: note = n is a, c, g, or t
  misc_feature 1431: note = n is a, c, g, or t
  misc_feature 1434: note = n is a, c, g, or t
  misc_feature 1437: note = n is a, c, g, or t
  misc_feature 1446: note = n is a, c, g, or t
  misc_feature 1449: note = n is a, c, g, or t
  misc_feature 1452: note = n is a, c, g, or t
  misc_feature 1461: note = n is a, c, g, or t
  misc_feature 1467: note = n is a, c, g, or t
  misc_feature 1470: note = n is a, c, g, or t
  misc_feature 1479: note = n is a, c, g, or t
  misc_feature 1500: note = n is a, c, g, or t
  misc_feature 1506: note = n is a, c, g, or t
  misc_feature 1527: note = n is a, c, g, or t
  misc_feature 1530: note = n is a, c, g, or t
  misc_feature 1533: note = n is a, c, g, or t
  misc_feature 1539: note = n is a, c, g, or t
  misc_feature 1542: note = n is a, c, g, or t
  misc_feature 1545: note = n is a, c, g, or t
  misc_feature 1554: note = n is a, c, g, or t
  misc_feature 1557: note = n is a, c, g, or t
  misc_feature 1560: note = n is a, c, g, or t
  misc_feature 1563: note = n is a, c, g, or t
  misc_feature 1572: note = n is a, c, g, or t
  misc_feature 1575: note = n is a, c, g, or t
  misc_feature 1578: note = n is a, c, g, or t
  misc_feature 1587: note = n is a, c, g, or t
  misc_feature 1590: note = n is a, c, g, or t
  misc_feature 1593: note = n is a, c, g, or t
  misc_feature 1596: note = n is a, c, g, or t
  misc_feature 1599: note = n is a, c, g, or t
  misc_feature 1611: note = n is a, c, g, or t
  misc_feature 1617: note = n is a, c, g, or t
  misc_feature 1620: note = n is a, c, g, or t
  misc_feature 1623: note = n is a, c, g, or t
  misc_feature 1629: note = n is a, c, g, or t
  misc_feature 1635: note = n is a, c, g, or t
  misc_feature 1647: note = n is a, c, g, or t
  misc_feature 1653: note = n is a, c, g, or t
  misc_feature 1659: note = n is a, c, g, or t
  misc_feature 1662: note = n is a, c, g, or t
  misc_feature 1671: note = n is a, c, g, or t
  misc_feature 1674: note = n is a, c, g, or t
  misc_feature 1683: note = n is a, c, g, or t
  misc_feature 1686: note = n is a, c, g, or t
  misc_feature 1692: note = n is a, c, g, or t
  misc_feature 1698: note = n is a, c, g, or t
  misc_feature 1701: note = n is a, c, g, or t
  misc_feature 1704: note = n is a, c, g, or t
  misc_feature 1719: note = n is a, c, g, or t
  misc_feature 1722: note = n is a, c, g, or t
  misc_feature 1728: note = n is a, c, g, or t
  misc_feature 1731: note = n is a, c, g, or t
  misc_feature 1734: note = n is a, c, g, or t
  misc_feature 1743: note = n is a, c, g, or t
  misc_feature 1746: note = n is a, c, g, or t
  misc_feature 1749: note = n is a, c, g, or t
  misc_feature 1755: note = n is a, c, g, or t
  misc_feature 1761: note = n is a, c, g, or t
  misc_feature 1776: note = n is a, c, g, or t
  misc_feature 1779: note = n is a, c, g, or t
  misc_feature 1782: note = n is a, c, g, or t
  misc_feature 1788: note = n is a, c, g, or t
  misc_feature 1791: note = n is a, c, g, or t
  misc_feature 1797: note = n is a, c, g, or t
  misc_feature 1800: note = n is a, c, g, or t
  misc_feature 1809: note = n is a, c, g, or t
  misc_feature 1812: note = n is a, c, g, or t
  misc_feature 1815: note = n is a, c, g, or t
  misc_feature 1833: note = n is a, c, g, or t
  misc_feature 1839: note = n is a, c, g, or t
  misc_feature 1842: note = n is a, c, g, or t
  misc_feature 1851: note = n is a, c, g, or t
  misc_feature 1857: note = n is a, c, g, or t
  misc_feature 1863: note = n is a, c, g, or t
  misc_feature 1872: note = n is a, c, g, or t
  misc_feature 1875: note = n is a, c, g, or t
  misc_feature 1884: note = n is a, c, g, or t
  misc_feature 1887: note = n is a, c, g, or t
  misc_feature 1890: note = n is a, c, g, or t
  misc_feature 1896: note = n is a, c, g, or t
  misc_feature 1899: note = n is a, c, g, or t
  misc_feature 1902: note = n is a, c, g, or t
  misc_feature 1911: note = n is a, c, g, or t
  misc_feature 1914: note = n is a, c, g, or t
  misc_feature 1923: note = n is a, c, g, or t
  misc_feature 1926: note = n is a, c, g, or t
  misc_feature 1932: note = n is a, c, g, or t
  misc_feature 1935: note = n is a, c, g, or t
  misc_feature 1944: note = n is a, c, g, or t
  misc_feature 1956: note = n is a, c, g, or t
  misc_feature 1959: note = n is a, c, g, or t
  misc_feature 1965: note = n is a, c, g, or t
  misc_feature 1968: note = n is a, c, g, or t
  misc_feature 1971: note = n is a, c, g, or t
  misc_feature 1980: note = n is a, c, g, or t
  misc_feature 1983: note = n is a, c, g, or t
  misc_feature 1992: note = n is a, c, g, or t
  misc_feature 1995: note = n is a, c, g, or t
  misc_feature 1998: note = n is a, c, g, or t
  misc_feature 2001: note = n is a, c, g, or t
  misc_feature 2004: note = n is a, c, g, or t
  misc_feature 2007: note = n is a, c, g, or t
  misc_feature 2016: note = n is a, c, g, or t
  misc_feature 2025: note = n is a, c, g, or t
  misc_feature 2031: note = n is a, c, g, or t
  misc_feature 2046: note = n is a, c, g, or t
  misc_feature 2052: note = n is a, c, g, or t
  misc_feature 2058: note = n is a, c, g, or t
  misc_feature 2061: note = n is a, c, g, or t
  misc_feature 2073: note = n is a, c, g, or t
  misc_feature 2088: note = n is a, c, g, or t
  misc_feature 2091: note = n is a, c, g, or t
  misc_feature 2097: note = n is a, c, g, or t
  misc_feature 2100: note = n is a, c, g, or t
  misc_feature 2109: note = n is a, c, g, or t
  misc_feature 2118: note = n is a, c, g, or t
  misc_feature 2121: note = n is a, c, g, or t
  misc_feature 2130: note = n is a, c, g, or t
  misc_feature 2136: note = n is a, c, g, or t
  misc_feature 2142: note = n is a, c, g, or t
  misc_feature 2145: note = n is a, c, g, or t
  misc_feature 2151: note = n is a, c, g, or t
  misc_feature 2157: note = n is a, c, g, or t
  misc_feature 2166: note = n is a, c, g, or t
  misc_feature 2169: note = n is a, c, g, or t
  misc_feature 2178: note = n is a, c, g, or t
  misc_feature 2181: note = n is a, c, g, or t
  misc_feature 2184: note = n is a, c, g, or t
  misc_feature 2190: note = n is a, c, g, or t
  misc_feature 2193: note = n is a, c, g, or t
  misc_feature 2202: note = n is a, c, g, or t
  misc_feature 2205: note = n is a, c, g, or t
  misc_feature 2217: note = n is a, c, g, or t
  misc_feature 2226: note = n is a, c, g, or t
  misc_feature 2232: note = n is a, c, g, or t
  misc_feature 2235: note = n is a, c, g, or t
  misc_feature 2238: note = n is a, c, g, or t
  misc_feature 2241: note = n is a, c, g, or t
  misc_feature 2250: note = n is a, c, g, or t
  misc_feature 2253: note = n is a, c, g, or t
  misc_feature 2256: note = n is a, c, g, or t
  misc_feature 2268: note = n is a, c, g, or t
  misc_feature 2271: note = n is a, c, g, or t
  misc_feature 2277: note = n is a, c, g, or t
  misc_feature 2286: note = n is a, c, g, or t
  misc_feature 2289: note = n is a, c, g, or t
  misc_feature 2292: note = n is a, c, g, or t
  misc_feature 2298: note = n is a, c, g, or t
  misc_feature 2301: note = n is a, c, g, or t
  misc_feature 2304: note = n is a, c, g, or t
  misc_feature 2307: note = n is a, c, g, or t
  misc_feature 2310: note = n is a, c, g, or t
  misc_feature 2319: note = n is a, c, g, or t
  misc_feature 2322: note = n is a, c, g, or t
  misc_feature 2325: note = n is a, c, g, or t
  misc_feature 2334: note = n is a, c, g, or t
  misc_feature 2337: note = n is a, c, g, or t
  misc_feature 2340: note = n is a, c, g, or t
  misc_feature 2343: note = n is a, c, g, or t
  misc_feature 2352: note = n is a, c, g, or t
  misc_feature 2358: note = n is a, c, g, or t
  misc_feature 2361: note = n is a, c, g, or t
  misc_feature 2370: note = n is a, c, g, or t
  misc_feature 2373: note = n is a, c, g, or t
  misc_feature 2379: note = n is a, c, g, or t
  misc_feature 2382: note = n is a, c, g, or t
  misc_feature 2385: note = n is a, c, g, or t
  misc_feature 2394: note = n is a, c, g, or t
  misc_feature 2397: note = n is a, c, g, or t
  misc_feature 2400: note = n is a, c, g, or t
  misc_feature 2406: note = n is a, c, g, or t
  misc_feature 2415: note = n is a, c, g, or t
  misc_feature 2421: note = n is a, c, g, or t
  misc_feature 2430: note = n is a, c, g, or t
  misc_feature 2433: note = n is a, c, g, or t
  misc_feature 2436: note = n is a, c, g, or t
  misc_feature 2439: note = n is a, c, g, or t
  misc_feature 2451: note = n is a, c, g, or t
  misc_feature 2454: note = n is a, c, g, or t
  misc_feature 2457: note = n is a, c, g, or t
  misc_feature 2460: note = n is a, c, g, or t
  misc_feature 2463: note = n is a, c, g, or t
  misc_feature 2469: note = n is a, c, g, or t
  misc_feature 2472: note = n is a, c, g, or t
  misc_feature 2481: note = n is a, c, g, or t
  misc_feature 2484: note = n is a, c, g, or t
  misc_feature 2490: note = n is a, c, g, or t
  misc_feature 2493: note = n is a, c, g, or t
  misc_feature 2502: note = n is a, c, g, or t
  misc_feature 2505: note = n is a, c, g, or t
  misc_feature 2526: note = n is a, c, g, or t
  misc_feature 2532: note = n is a, c, g, or t
  misc_feature 2535: note = n is a, c, g, or t
  misc_feature 2550: note = n is a, c, g, or t
  misc_feature 2559: note = n is a, c, g, or t
  misc_feature 2565: note = n is a, c, g, or t
  misc_feature 2574: note = n is a, c, g, or t
  misc_feature 2577: note = n is a, c, g, or t
  misc_feature 2580: note = n is a, c, g, or t
  misc_feature 2586: note = n is a, c, g, or t
  misc_feature 2595: note = n is a, c, g, or t
  misc_feature 2598: note = n is a, c, g, or t
  misc_feature 2604: note = n is a, c, g, or t
  misc_feature 2607: note = n is a, c, g, or t
  misc_feature 2613: note = n is a, c, g, or t
  misc_feature 2616: note = n is a, c, g, or t
  misc_feature 2619: note = n is a, c, g, or t
  misc_feature 2628: note = n is a, c, g, or t
  misc_feature 2631: note = n is a, c, g, or t
  misc_feature 2634: note = n is a, c, g, or t
  misc_feature 2637: note = n is a, c, g, or t
  misc_feature 2646: note = n is a, c, g, or t
  misc_feature 2649: note = n is a, c, g, or t
  misc_feature 2655: note = n is a, c, g, or t
  misc_feature 2661: note = n is a, c, g, or t
  misc_feature 2664: note = n is a, c, g, or t
  misc_feature 2667: note = n is a, c, g, or t
  misc_feature 2670: note = n is a, c, g, or t
  misc_feature 2673: note = n is a, c, g, or t
  misc_feature 2676: note = n is a, c, g, or t
  misc_feature 2679: note = n is a, c, g, or t
  misc_feature 2688: note = n is a, c, g, or t
  misc_feature 2691: note = n is a, c, g, or t
  misc_feature 2706: note = n is a, c, g, or t
  misc_feature 2724: note = n is a, c, g, or t
  misc_feature 2736: note = n is a, c, g, or t
  misc_feature 2754: note = n is a, c, g, or t
  misc_feature 2757: note = n is a, c, g, or t
  misc_feature 2760: note = n is a, c, g, or t
  misc_feature 2772: note = n is a, c, g, or t
  misc_feature 2775: note = n is a, c, g, or t
  misc_feature 2796: note = n is a, c, g, or t
  misc_feature 2799: note = n is a, c, g, or t
  misc_feature 2805: note = n is a, c, g, or t
  misc_feature 2808: note = n is a, c, g, or t
  misc_feature 2811: note = n is a, c, g, or t
  misc_feature 2817: note = n is a, c, g, or t
  misc_feature 2829: note = n is a, c, g, or t
  misc_feature 2838: note = n is a, c, g, or t
  misc_feature 2841: note = n is a, c, g, or t
  misc_feature 2847: note = n is a, c, g, or t
  misc_feature 2856: note = n is a, c, g, or t
  misc_feature 2859: note = n is a, c, g, or t
  misc_feature 2862: note = n is a, c, g, or t
  misc_feature 2865: note = n is a, c, g, or t
  misc_feature 2868: note = n is a, c, g, or t
  misc_feature 2871: note = n is a, c, g, or t
  misc_feature 2877: note = n is a, c, g, or t
  misc_feature 2880: note = n is a, c, g, or t
  misc_feature 2883: note = n is a, c, g, or t
  misc_feature 2886: note = n is a, c, g, or t
  misc_feature 2895: note = n is a, c, g, or t
  misc_feature 2898: note = n is a, c, g, or t
  misc_feature 2907: note = n is a, c, g, or t
  misc_feature 2910: note = n is a, c, g, or t
  misc_feature 2919: note = n is a, c, g, or t
  misc_feature 2925: note = n is a, c, g, or t
  misc_feature 2940: note = n is a, c, g, or t
  misc_feature 2946: note = n is a, c, g, or t
  misc_feature 2958: note = n is a, c, g, or t
  misc_feature 2961: note = n is a, c, g, or t
  misc_feature 2964: note = n is a, c, g, or t
  misc_feature 2967: note = n is a, c, g, or t
  misc_feature 2970: note = n is a, c, g, or t
  misc_feature 2973: note = n is a, c, g, or t
  misc_feature 2988: note = n is a, c, g, or t
  misc_feature 2991: note = n is a, c, g, or t
  misc_feature 2994: note = n is a, c, g, or t
  misc_feature 2997: note = n is a, c, g, or t
  misc_feature 3000: note = n is a, c, g, or t
  misc_feature 3003: note = n is a, c, g, or t
  misc_feature 3006: note = n is a, c, g, or t
  misc_feature 3015: note = n is a, c, g, or t
  misc_feature 3027: note = n is a, c, g, or t
  misc_feature 3030: note = n is a, c, g, or t
  misc_feature 3036: note = n is a, c, g, or t
  misc_feature 3039: note = n is a, c, g, or t
  misc_feature 3045: note = n is a, c, g, or t
  misc_feature 3048: note = n is a, c, g, or t
  misc_feature 3051: note = n is a, c, g, or t
  misc_feature 3057: note = n is a, c, g, or t
  misc_feature 3060: note = n is a, c, g, or t
  misc_feature 3063: note = n is a, c, g, or t
  misc_feature 3075: note = n is a, c, g, or t
  misc_feature 3078: note = n is a, c, g, or t
  misc_feature 3081: note = n is a, c, g, or t
  misc_feature 3084: note = n is a, c, g, or t
  misc_feature 3087: note = n is a, c, g, or t
  misc_feature 3102: note = n is a, c, g, or t
  misc_feature 3114: note = n is a, c, g, or t
  misc_feature 3126: note = n is a, c, g, or t
  misc_feature 3129: note = n is a, c, g, or t
  misc_feature 3132: note = n is a, c, g, or t
  misc_feature 3138: note = n is a, c, g, or t
  misc_feature 3150: note = n is a, c, g, or t
  misc_feature 3153: note = n is a, c, g, or t
  misc_feature 3159: note = n is a, c, g, or t
  misc_feature 3165: note = n is a, c, g, or t
  misc_feature 3171: note = n is a, c, g, or t
  misc_feature 3177: note = n is a, c, g, or t
  misc_feature 3180: note = n is a, c, g, or t
  misc_feature 3183: note = n is a, c, g, or t
  misc_feature 3186: note = n is a, c, g, or t
  misc_feature 3192: note = n is a, c, g, or t
  misc_feature 3195: note = n is a, c, g, or t
  misc_feature 3198: note = n is a, c, g, or t
  misc_feature 3201: note = n is a, c, g, or t
  misc_feature 3204: note = n is a, c, g, or t
  misc_feature 3210: note = n is a, c, g, or t
  misc_feature 3213: note = n is a, c, g, or t
  misc_feature 3225: note = n is a, c, g, or t
  misc_feature 3252: note = n is a, c, g, or t
  misc_feature 3261: note = n is a, c, g, or t
  misc_feature 3267: note = n is a, c, g, or t
  misc_feature 3273: note = n is a, c, g, or t
  misc_feature 3276: note = n is a, c, g, or t
  misc_feature 3279: note = n is a, c, g, or t
  misc_feature 3309: note = n is a, c, g, or t
  misc_feature 3321: note = n is a, c, g, or t
  misc_feature 3324: note = n is a, c, g, or t
  misc_feature 3327: note = n is a, c, g, or t
  misc_feature 3330: note = n is a, c, g, or t
  misc_feature 3333: note = n is a, c, g, or t
  misc_feature 3339: note = n is a, c, g, or t
  misc_feature 3357: note = n is a, c, g, or t
  misc_feature 3363: note = n is a, c, g, or t
  misc_feature 3375: note = n is a, c, g, or t
  misc_feature 3378: note = n is a, c, g, or t
  misc_feature 3381: note = n is a, c, g, or t
  misc_feature 3384: note = n is a, c, g, or t
  misc_feature 3387: note = n is a, c, g, or t
  misc_feature 3390: note = n is a, c, g, or t
  source 1..3393: mol_type = other DNA
atgytntggtgggargargtngargaytgytaygarmgngargaygtncaraaraaracnttyacnaartgggtnaaygcncarttywsnaarttyggnaarcarcayathgaraayytnttywsngayytncargayggnmgnmgnytnytngayytnytngarggnytnacnggncaraarytnccnaargaraarggnwsnacnmgngtncaygcnytnaayaaygtnaayaargcnytnmgngtnytncaraayaayaaygtngayytngtnaayathggnwsnacngayathgtngayggnaaycayaarytnacnytnggnytnathtggaayathathytncaytggcargtnaaraaygtnatgaaraayathatggcnggnytncarcaracnaaywsngaraarathytnytnwsntgggtnmgncarwsnacnmgnaaytayccncargtnaaygtnathaayttyacnacnwsntggwsngayggnytngcnytnaaygcnytnathcaywsncaymgnccngayytnttygaytggaaywsngtngtntgycarcarwsngcnacncarmgnytngarcaygcnttyaayathgcnmgntaycarytnggnathgaraarytnytngayccngargaygtngayacnacntayccngayaaraarwsnathytnatgtayathacnwsnytnttycargtnytnccncarcargtnwsnathgargcnathcargargtngaratgytnccnmgnccnccnaargtnacnaargargarcayttycarytncaycaycaratgcaytaywsncarcarathacngtnwsnytngcncarggntaygarmgnacnwsnwsnccnaarccnmgnttyaarwsntaygcntayacncargcngcntaygtnacnacnwsngayccnacnmgnwsnccnttyccnwsncarcayytngargcnccngargayaarwsnttyggnwsnwsnytnatggarwsngargtnaayytngaymgntaycaracngcnytngargargtnytnwsntggytnytnwsngcngargayacnytncargcncarggngarathwsnaaygaygtngargtngtnaargaycarttycayacncaygarggntayatgatggayytnacngcncaycarggnmgngtnggnaayathytncarytnggnwsnaarytnathggnacnggnaarytnwsngargaygargaracngargtncargarcaratgaayytnytnaaywsnmgntgggartgyytnmgngtngcnwsnatggaraarcarwsnaayytncaymgngtnytnatggayytncaraaycaraarytnaargarytnaaygaytggytnacngargcngaracnacngcnaaygtnytncargaygcnacnmgnaargarmgnytnytngargaywsnaarggngtnaargarytnatgaarcartggcargayytncarggngarathgarcargarcargtnmgngtnaaywsnytnacncayatggtngtngtngtngaygarwsnwsnggngaycaygcnacngcngcnytngargarcarytnaargtnytnggngaymgntgggcnaayathtgymgntggwsngarytnmgnaaraarwsnytnaayathmgnwsncayytngargcnwsnwsngaycartggaarmgnytncayytnwsnytncargarytnytngtntggytncarytnaargaygaygarytnwsnmgncargcnccnathggnggngayttyccngcngtncaraarcaraaygaygtncaymgngcnttyaarmgngarytnaaracnaargarccngtnathatgwsnacnytngaracngtnmgnathttyytnacngarcarccnytngarggnytngaraarytntaycargarccnmgngarytnccnccngargarmgngcncaraaygtnacnmgnytnytnmgnaarcargcngargargtnaayacngartgggaraarytnaayytncaywsngcngaytggcarmgnaarathgaygaracnytngarmgnytncargarytncargargcnacngaygarytngayytnaarytnmgncargcngargtnathaarggnwsntggcarccngtnggngayytnytnathgaywsnytncargaycayytngaraargtnaargcnytnmgnggngarathgcnccnytnaargaraaygtnwsncaygtnaaygayytngcnmgncarytnacnacnytnggnathcarytnwsnccntayaayytnwsnacnytngargayytnaayacnmgntggaarytnytncargtngcngtngargaymgngtnmgncarytncaygargcncaymgngayttyggnccngcnwsncarcayttyytnwsnacnwsngtncarggnccntgggarmgngcnathwsnccnaayaargtnccntaytayathaaycaygaracncaracnacntgytgggaycayccnaaratgacngarytntaycarwsnytngcngayytnaayaaygtnmgnttywsngcntaymgnacngcnatgaarytnmgnmgnytncaraargcnytntgyytngayytnytnwsnytnwsngcngcntgygaygcnytngaycarcayaayytnaarcaraaygaycarccnatggayathytncarathathaaytgyytnacnacnathtaygaymgnytngarcargarcayaayaayytngtnaaygtnccnytntgygtngayatgtgyytnaaytggytnytnaaygtntaygayacnggnmgnacnggnmgnathmgngtnytnwsnttyaaracnggnathathwsnytntgyaargcncayytngargayaartaymgntayytnttyaarcargtngcnwsnwsnacnggnttytgygaycarmgnmgnytnggnytnytnytncaygaywsnathcarathccnmgncarytnggngargtngcnwsnttyggnggnwsnaayathgarccnwsngtnmgnwsntgyttycarttygcnaayaayaarccngarathgargcngcnytnttyytngaytggatgmgnytngarccncarwsnatggtntggytnccngtnytncaymgngtngcngcngcngaracngcnaarcaycargcnaartgyaayathtgyaargartgyccnathathggnttymgntaymgnwsnytnaarcayttyaaytaygayathtgycarwsntgyttyttywsnggnmgngtngcnaarggncayaaratgcaytayccnatggtngartaytgyacnccnacnacnwsnggngar

SEQ ID NO: 3 - protein, 1098 aa, synthetic construct
  REGION 1..1098: note = amino acid sequence of nano-utrophin 1
  REGION 1..433: note = MISC_FEATURE - amino acid sequence identical to N-terminal region of full length human utrophin
  SITE 433: note = MISC_FEATURE - tryptophan residue in the "A" helix at the core of the Hidden Markov Model (HMM) and all crystal structures for proteins in this superfamily
  REGION 434..477: note = MISC_FEATURE - amino acid sequence identical to C-terminal region of full length human utrophin
  REGION 477..478: note = MISC_FEATURE - positions within the superfamily HMM for the "B" helix that flank the hypothetical plane of transection as depicted in the FIG 2F
  REGION 478..514: note = MISC_FEATURE - amino acid sequence identical to N-terminal region of full length human utrophin
  SITE 514: note = MISC_FEATURE - tryptophan residue in the "C" helix at the core of the Hidden Markov Model (HMM) and all crystal structures for proteins in this superfamily
  REGION 515..1098: note = MISC_FEATURE - amino acid sequence identical to C-terminal region of full length human utrophin
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWIQEAETTVNVLVDASHRENALQDSILARELKQQMQDIQAEIDAHQVKVNSLTHMVVIVDENSGESATAILEDQLQKLGERWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGE

SEQ ID NO: 4 - DNA, 3294 bp, synthetic construct
  misc_feature 1..3294: note = nucleic acid sequence encoding nano-utrophin-1 protein
  misc_feature 6: note = n is a, c, g, or t
  misc_feature 15: note = n is a, c, g, or t
  misc_feature 27: note = n is a, c, g, or t
  misc_feature 30: note = n is a, c, g, or t
  misc_feature 33: note = n is a, c, g, or t
  misc_feature 42: note = n is a, c, g, or t
  misc_feature 57: note = n is a, c, g, or t
  misc_feature 72: note = n is a, c, g, or t
  misc_feature 75: note = n is a, c, g, or t
  misc_feature 78: note = n is a, c, g, or t
  misc_feature 96: note = n is a, c, g, or t
  misc_feature 108: note = n is a, c, g, or t
  misc_feature 114: note = n is a, c, g, or t
  misc_feature 129: note = n is a, c, g, or t
  misc_feature 132: note = n is a, c, g, or t
  misc_feature 138: note = n is a, c, g, or t
  misc_feature 144: note = n is a, c, g, or t
  misc_feature 147: note = n is a, c, g, or t
  misc_feature 153: note = n is a, c, g, or t
  misc_feature 156: note = n is a, c, g, or t
  misc_feature 174: note = n is a, c, g, or t
  misc_feature 180: note = n is a, c, g, or t
  misc_feature 189: note = n is a, c, g, or t
  misc_feature 192: note = n is a, c, g, or t
  misc_feature 198: note = n is a, c, g, or t
  misc_feature 201: note = n is a, c, g, or t
  misc_feature 207: note = n is a, c, g, or t
  misc_feature 210: note = n is a, c, g, or t
  misc_feature 216: note = n is a, c, g, or t
  misc_feature 219: note = n is a, c, g, or t
  misc_feature 222: note = n is a, c, g, or t
  misc_feature 225: note = n is a, c, g, or t
  misc_feature 228: note = n is a, c, g, or t
  misc_feature 231: note = n is a, c, g, or t
  misc_feature 234: note = n is a, c, g, or t
  misc_feature 237: note = n is a, c, g, or t
  misc_feature 246: note = n is a, c, g, or t
  misc_feature 249: note = n is a, c, g, or t
  misc_feature 252: note = n is a, c, g, or t
  misc_feature 255: note = n is a, c, g, or t
  misc_feature 258: note = n is a, c, g, or t
  misc_feature 261: note = n is a, c, g, or t
  misc_feature 267: note = n is a, c, g, or t
  misc_feature 270: note = n is a, c, g, or t
  misc_feature 279: note = n is a, c, g, or t
  misc_feature 285: note = n is a, c, g, or t
  misc_feature 288: note = n is a, c, g, or t
  misc_feature 291: note = n is a, c, g, or t
  misc_feature 297: note = n is a, c, g, or t
  misc_feature 300: note = n is a, c, g, or t
  misc_feature 315: note = n is a, c, g, or t
  misc_feature 321: note = n is a, c, g, or t
  misc_feature 324: note = n is a, c, g, or t
  misc_feature 333: note = n is a, c, g, or t
  misc_feature 336: note = n is a, c, g, or t
  misc_feature 339: note = n is a, c, g, or t
  misc_feature 348: note = n is a, c, g, or t
  misc_feature 354: note = n is a, c, g, or t
  misc_feature 366: note = n is a, c, g, or t
  misc_feature 369: note = n is a, c, g, or t
  misc_feature 372: note = n is a, c, g, or t
  misc_feature 375: note = n is a, c, g, or t
  misc_feature 378: note = n is a, c, g, or t
  misc_feature 381: note = n is a, c, g, or t
  misc_feature 387: note = n is a, c, g, or t
  misc_feature 396: note = n is a, c, g, or t
  misc_feature 408: note = n is a, c, g, or t
  misc_feature 417: note = n is a, c, g, or t
  misc_feature 429: note = n is a, c, g, or t
  misc_feature 435: note = n is a, c, g, or t
  misc_feature 441: note = n is a, c, g, or t
  misc_feature 450: note = n is a, c, g, or t
  misc_feature 456: note = n is a, c, g, or t
  misc_feature 468: note = n is a, c, g, or t
  misc_feature 471: note = n is a, c, g, or t
  misc_feature 474: note = n is a, c, g, or t
  misc_feature 480: note = n is a, c, g, or t
  misc_feature 483: note = n is a, c, g, or t
  misc_feature 489: note = n is a, c, g, or t
  misc_feature 492: note = n is a, c, g, or t
  misc_feature 495: note = n is a, c, g, or t
  misc_feature 498: note = n is a, c, g, or t
  misc_feature 504: note = n is a, c, g, or t
  misc_feature 510: note = n is a, c, g, or t
  misc_feature 516: note = n is a, c, g, or t
  misc_feature 519: note = n is a, c, g, or t
  misc_feature 528: note = n is a, c, g, or t
  misc_feature 531: note = n is a, c, g, or t
  misc_feature 534: note = n is a, c, g, or t
  misc_feature 540: note = n is a, c, g, or t
  misc_feature 546: note = n is a, c, g, or t
  misc_feature 549: note = n is a, c, g, or t
  misc_feature 552: note = n is a, c, g, or t
  misc_feature 561: note = n is a, c, g, or t
  misc_feature 564: note = n is a, c, g, or t
  misc_feature 567: note = n is a, c, g, or t
  misc_feature 573: note = n is a, c, g, or t
  misc_feature 582: note = n is a, c, g, or t
  misc_feature 588: note = n is a, c, g, or t
  misc_feature 594: note = n is a, c, g, or t
  misc_feature 606: note = n is a, c, g, or t
  misc_feature 609: note = n is a, c, g, or t
  misc_feature 618: note = n is a, c, g, or t
  misc_feature 621: note = n is a, c, g, or t
  misc_feature 630: note = n is a, c, g, or t
  misc_feature 633: note = n is a, c, g, or t
  misc_feature 642: note = n is a, c, g, or t
  misc_feature 648: note = n is a, c, g, or t
  misc_feature 654: note = n is a, c, g, or t
  misc_feature 660: note = n is a, c, g, or t
  misc_feature 666: note = n is a, c, g, or t
  misc_feature 669: note = n is a, c, g, or t
  misc_feature 681: note = n is a, c, g, or t
  misc_feature 684: note = n is a, c, g, or t
  misc_feature 690: note = n is a, c, g, or t
  misc_feature 699: note = n is a, c, g, or t
  misc_feature 702: note = n is a, c, g, or t
  misc_feature 705: note = n is a, c, g, or t
  misc_feature 711: note = n is a, c, g, or t
  misc_feature 714: note = n is a, c, g, or t
  misc_feature 726: note = n is a, c, g, or t
  misc_feature 741: note = n is a, c, g, or t
  misc_feature 744: note = n is a, c, g, or t
  misc_feature 747: note = n is a, c, g, or t
  misc_feature 750: note = n is a, c, g, or t
  misc_feature 759: note = n is a, c, g, or t
  misc_feature 762: note = n is a, c, g, or t
  misc_feature 765: note = n is a, c, g, or t
  misc_feature 774: note = n is a, c, g, or t
  misc_feature 777: note = n is a, c, g, or t
  misc_feature 786: note = n is a, c, g, or t
  misc_feature 792: note = n is a, c, g, or t
  misc_feature 798: note = n is a, c, g, or t
  misc_feature 804: note = n is a, c, g, or t
  misc_feature 807: note = n is a, c, g, or t
  misc_feature 810: note = n is a, c, g, or t
  misc_feature 813: note = n is a, c, g, or t
  misc_feature 843: note = n is a, c, g, or t
  misc_feature 858: note = n is a, c, g, or t
  misc_feature 861: note = n is a, c, g, or t
  misc_feature 864: note = n is a, c, g, or t
  misc_feature 867: note = n is a, c, g, or t
  misc_feature 885: note = n is a, c, g, or t
  misc_feature 888: note = n is a, c, g, or t
  misc_feature 891: note = n is a, c, g, or t
  misc_feature 894: note = n is a, c, g, or t
  misc_feature 900: note = n is a, c, g, or t
  misc_feature 903: note = n is a, c, g, or t
  misc_feature 906: note = n is a, c, g, or t
  misc_feature 909: note = n is a, c, g, or t
  misc_feature 912: note = n is a, c, g, or t
  misc_feature 915: note = n is a, c, g, or t
  misc_feature 921: note = n is a, c, g, or t
  misc_feature 933: note = n is a, c, g, or t
  misc_feature 939: note = n is a, c, g, or t
  misc_feature 951: note = n is a, c, g, or t
  misc_feature 954: note = n is a, c, g, or t
  misc_feature 963: note = n is a, c, g, or t
  misc_feature 966: note = n is a, c, g, or t
  misc_feature 969: note = n is a, c, g, or t
  misc_feature 975: note = n is a, c, g, or t
  misc_feature 978: note = n is a, c, g, or t
  misc_feature 981: note = n is a, c, g, or t
  misc_feature 984: note = n is a, c, g, or t
  misc_feature 993: note = n is a, c, g, or t
  misc_feature 1017: note = n is a, c, g, or t
  misc_feature 1026: note = n is a, c, g, or t
  misc_feature 1035: note = n is a, c, g, or t
  misc_feature 1050: note = n is a, c, g, or t
  misc_feature 1053: note = n is a, c, g, or t
  misc_feature 1062: note = n is a, c, g, or t
  misc_feature 1077: note = n is a, c, g, or t
  misc_feature 1080: note = n is a, c, g, or t
  misc_feature 1083: note = n is a, c, g, or t
  misc_feature 1092: note = n is a, c, g, or t
  misc_feature 1095: note = n is a, c, g, or t
  misc_feature 1098: note = n is a, c, g, or t
  misc_feature 1101: note = n is a, c, g, or t
  misc_feature 1104: note = n is a, c, g, or t
  misc_feature 1107: note = n is a, c, g, or t
  misc_feature 1110: note = n is a, c, g, or t
  misc_feature 1116: note = n is a, c, g, or t
  misc_feature 1119: note = n is a, c, g, or t
  misc_feature 1128: note = n is a, c, g, or t
  misc_feature 1134: note = n is a, c, g, or t
  misc_feature 1140: note = n is a, c, g, or t
  misc_feature 1143: note = n is a, c, g, or t
  misc_feature 1146: note = n is a, c, g, or t
  misc_feature 1149: note = n is a, c, g, or t
  misc_feature 1185: note = n is a, c, g, or t
  misc_feature 1188: note = n is a, c, g, or t
  misc_feature 1191: note = n is a, c, g, or t
  misc_feature 1197: note = n is a, c, g, or t
  misc_feature 1200: note = n is a, c, g, or t
  misc_feature 1209: note = n is a, c, g, or t
  misc_feature 1212: note = n is a, c, g, or t
  misc_feature 1215: note = n is a, c, g, or t
  misc_feature 1218: note = n is a, c, g, or t
  misc_feature 1224: note = n is a, c, g, or t
  misc_feature 1233: note = n is a, c, g, or t
  misc_feature 1239: note = n is a, c, g, or t
  misc_feature 1242: note = n is a, c, g, or t
  misc_feature 1245: note = n is a, c, g, or t
  misc_feature 1254: note = n is a, c, g, or t
  misc_feature 1257: note = n is a, c, g, or t
  misc_feature 1266: note = n is a, c, g, or t
  misc_feature 1281: note = n is a, c, g, or t
  misc_feature 1290: note = n is a, c, g, or t
  misc_feature 1293: note = n is a, c, g, or t
  misc_feature 1296: note = n is a, c, g, or t
  misc_feature 1311: note = n is a, c, g, or t
  misc_feature 1317: note = n is a, c, g, or t
  misc_feature 1320: note = n is a, c, g, or t
  misc_feature 1323: note = n is a, c, g, or t
  misc_feature 1329: note = n is a, c, g, or t
  misc_feature 1332: note = n is a, c, g, or t
  misc_feature 1335: note = n is a, c, g, or t
  misc_feature 1341: note = n is a, c, g, or t
  misc_feature 1344: note = n is a, c, g, or t
  misc_feature 1350: note = n is a, c, g, or t
  misc_feature 1359: note = n is a, c, g, or t
  misc_feature 1362: note = n is a, c, g, or t
  misc_feature 1371: note = n is a, c, g, or t
  misc_feature 1377: note = n is a, c, g, or t
  misc_feature 1380: note = n is a, c, g, or t
  misc_feature 1383: note = n is a, c, g, or t
  misc_feature 1389: note = n is a, c, g, or t
  misc_feature 1416: note = n is a, c, g, or t
  misc_feature 1428: note = n is a, c, g, or t
  misc_feature 1437: note = n is a, c, g, or t
  misc_feature 1443: note = n is a, c, g, or t
  misc_feature 1449: note = n is a, c, g, or t
  misc_feature 1452: note = n is a, c, g, or t
  misc_feature 1455: note = n is a, c, g, or t
  misc_feature 1464: note = n is a, c, g, or t
  misc_feature 1467: note = n is a, c, g, or t
  misc_feature 1473: note = n is a, c, g, or t
  misc_feature 1485: note = n is a, c, g, or t
  misc_feature 1488: note = n is a, c, g, or t
  misc_feature 1494: note = n is a, c, g, or t
  misc_feature 1497: note = n is a, c, g, or t
  misc_feature 1500: note = n is a, c, g, or t
  misc_feature 1503: note = n is a, c, g, or t
  misc_feature 1509: note = n is a, c, g, or t
  misc_feature 1521: note = n is a, c, g, or t
  misc_feature 1530: note = n is a, c, g, or t
  misc_feature 1533: note = n is a, c, g, or t
  misc_feature 1539: note = n is a, c, g, or t
  misc_feature 1551: note = n is a, c, g, or t
  misc_feature 1557: note = n is a, c, g, or t
  misc_feature 1563: note = n is a, c, g, or t
  misc_feature 1566: note = n is a, c, g, or t
  misc_feature 1569: note = n is a, c, g, or t
  misc_feature 1575: note = n is a, c, g, or t
  misc_feature 1578: note = n is a, c, g, or t
  misc_feature 1584: note = n is a, c, g, or t
  misc_feature 1590: note = n is a, c, g, or t
  misc_feature 1593: note = n is a, c, g, or t
  misc_feature 1596: note = n is a, c, g, or t
  misc_feature 1611: note = n is a, c, g, or t
  misc_feature 1614: note = n is a, c, g, or t
  misc_feature 1617: note = n is a, c, g, or t
  misc_feature 1623: note = n is a, c, g, or t
  misc_feature 1626: note = n is a, c, g, or t
  misc_feature 1635: note = n is a, c, g, or t
  misc_feature 1647: note = n is a, c, g, or t
  misc_feature 1668: note = n is a, c, g, or t
  misc_feature 1683: note = n is a, c, g, or t
  misc_feature 1689: note = n is a, c, g, or t
  misc_feature 1692: note = n is a, c, g, or t
  misc_feature 1698: note = n is a, c, g, or t
  misc_feature 1701: note = n is a, c, g, or t
  misc_feature 1704: note = n is a, c, g, or t
  misc_feature 1707: note = n is a, c, g, or t
  misc_feature 1713: note = n is a, c, g, or t
  misc_feature 1734: note = n is a, c, g, or t
  misc_feature 1737: note = n is a, c, g, or t
  misc_feature 1740: note = n is a, c, g, or t
  misc_feature 1743: note = n is a, c, g, or t
  misc_feature 1749: note = n is a, c, g, or t
  misc_feature 1767: note = n is a, c, g, or t
  misc_feature 1770: note = n is a, c, g, or t
  misc_feature 1773: note = n is a, c, g, or t
  misc_feature 1779: note = n is a, c, g, or t
  misc_feature 1782: note = n is a, c, g, or t
  misc_feature 1791: note = n is a, c, g, or t
  misc_feature 1794: note = n is a, c, g, or t
  misc_feature 1797: note = n is a, c, g, or t
  misc_feature 1803: note = n is a, c, g, or t
  misc_feature 1806: note = n is a, c, g, or t
  misc_feature 1815: note = n is a, c, g, or t
  misc_feature 1824: note = n is a, c, g, or t
  misc_feature 1827: note = n is a, c, g, or t
  misc_feature 1836: note = n is a, c, g, or t
  misc_feature 1839: note = n is a, c, g, or t
  misc_feature 1842: note = n is a, c, g, or t
  misc_feature 1848: note = n is a, c, g, or t
  misc_feature 1854: note = n is a, c, g, or t
  misc_feature 1860: note = n is a, c, g, or t
  misc_feature 1866: note = n is a, c, g, or t
  misc_feature 1869: note = n is a, c, g, or t
  misc_feature 1872: note = n is a, c, g, or t
  misc_feature 1881: note = n is a, c, g, or t
  misc_feature 1884: note = n is a, c, g, or t
  misc_feature 1896: note = n is a, c, g, or t
  misc_feature 1902: note = n is a, c, g, or t
  misc_feature 1908: note = n is a, c, g, or t
  misc_feature 1917: note = n is a, c, g, or t
  misc_feature 1920: note = n is a, c, g, or t
  misc_feature 1926: note = n is a, c, g, or t
  misc_feature 1944: note = n is a, c, g, or t
  misc_feature 1947: note = n is a, c, g, or t
  misc_feature 1953: note = n is a, c, g, or t
  misc_feature 1956: note = n is a, c, g, or t
  misc_feature 1959: note = n is a, c, g, or t
  misc_feature 1962: note = n is a, c, g, or t
  misc_feature 1980: note = n is a, c, g, or t
  misc_feature 1989: note = n is a, c, g, or t
  misc_feature 1992: note = n is a, c, g, or t
  misc_feature 2001: note = n is a, c, g, or t
  misc_feature 2004: note = n is a, c, g, or t
  misc_feature 2010: note = n is a, c, g, or t
  misc_feature 2016: note = n is a, c, g, or t
  misc_feature 2019: note = n is a, c, g, or t
  misc_feature 2031: note = n is a, c, g, or t
  misc_feature 2037: note = n is a, c, g, or t
  misc_feature 2052: note = n is a, c, g, or t
  misc_feature 2058: note = n is a, c, g, or t
  misc_feature 2061: note = n is a, c, g, or t
  misc_feature 2064: note = n is a, c, g, or t
  misc_feature 2070: note = n is a, c, g, or t
  misc_feature 2079: note = n is a, c, g, or t
  misc_feature 2082: note = n is a, c, g, or t
  misc_feature 2085: note = n is a, c, g, or t
  misc_feature 2091: note = n is a, c, g, or t
  misc_feature 2094: note = n is a, c, g, or t
  misc_feature 2103: note = n is a, c, g, or t
  misc_feature 2106: note = n is a, c, g, or t
  misc_feature 2133: note = n is a, c, g, or t
  misc_feature 2139: note = n is a, c, g, or t
  misc_feature 2151: note = n is a, c, g, or t
  misc_feature 2154: note = n is a, c, g, or t
  misc_feature 2169: note = n is a, c, g, or t
  misc_feature 2175: note = n is a, c, g, or t
  misc_feature 2178: note = n is a, c, g, or t
  misc_feature 2187: note = n is a, c, g, or t
  misc_feature 2190: note = n is a, c, g, or t
  misc_feature 2193: note = n is a, c, g, or t
  misc_feature 2199: note = n is a, c, g, or t
  misc_feature 2202: note = n is a, c, g, or t
  misc_feature 2205: note = n is a, c, g, or t
  misc_feature 2208: note = n is a, c, g, or t
  misc_feature 2214: note = n is a, c, g, or t
  misc_feature 2220: note = n is a, c, g, or t
  misc_feature 2223: note = n is a, c, g, or t
  misc_feature 2226: note = n is a, c, g, or t
  misc_feature 2235: note = n is a, c, g, or t
  misc_feature 2238: note = n is a, c, g, or t
  misc_feature 2244: note = n is a, c, g, or t
  misc_feature 2253: note = n is a, c, g, or t
  misc_feature 2262: note = n is a, c, g, or t
  misc_feature 2271: note = n is a, c, g, or t
  misc_feature 2274: note = n is a, c, g, or t
  misc_feature 2280: note = n is a, c, g, or t
  misc_feature 2283: note = n is a, c, g, or t
  misc_feature 2286: note = n is a, c, g, or t
  misc_feature 2295: note = n is a, c, g, or t
  misc_feature 2298: note = n is a, c, g, or t
  misc_feature 2307: note = n is a, c, g, or t
  misc_feature 2316: note = n is a, c, g, or t
  misc_feature 2322: note = n is a, c, g, or t
  misc_feature 2331: note = n is a, c, g, or t
  misc_feature 2334: note = n is a, c, g, or t
  misc_feature 2337: note = n is a, c, g, or t
  misc_feature 2340: note = n is a, c, g, or t
  misc_feature 2352: note = n is a, c, g, or t
  misc_feature 2355: note = n is a, c, g, or t
  misc_feature 2358: note = n is a, c, g, or t
  misc_feature 2361: note = n is a, c, g, or t
  misc_feature 2364: note = n is a, c, g, or t
  misc_feature 2370: note = n is a, c, g, or t
  misc_feature 2373: note = n is a, c, g, or t
  misc_feature 2382: note = n is a, c, g, or t
  misc_feature 2385: note = n is a, c, g, or t
  misc_feature 2391: note = n is a, c, g, or t
  misc_feature 2403: note = n is a, c, g, or t
  misc_feature 2406: note = n is a, c, g, or t
  misc_feature 2427: note = n is a, c, g, or t
  misc_feature 2433: note = n is a, c, g, or t
  misc_feature 2436: note = n is a, c, g, or t
  misc_feature 2451: note = n is a, c, g, or t
  misc_feature 2460: note = n is a, c, g, or t
  misc_feature 2466: note = n is a, c, g, or t
  misc_feature 2475: note = n is a, c, g, or t
  misc_feature 2478: note = n is a, c, g, or t
  misc_feature 2481: note = n is a, c, g, or t
  misc_feature 2487: note = n is a, c, g, or t
  misc_feature 2496: note = n is a, c, g, or t
  misc_feature 2499: note = n is a, c, g, or t
  misc_feature 2505: note = n is a, c, g, or t
  misc_feature 2508: note = n is a, c, g, or t
  misc_feature 2514: note = n is a, c, g, or t
  misc_feature 2517: note = n is a, c, g, or t
  misc_feature 2520: note = n is a, c, g, or t
  misc_feature 2532: note = n is a, c, g, or t
  misc_feature 2535: note = n is a, c, g, or t
  misc_feature 2538: note = n is a, c, g, or t
  misc_feature 2547: note = n is a, c, g, or t
  misc_feature 2550: note = n is a, c, g, or t
  misc_feature 2556: note = n is a, c, g, or t
  misc_feature 2562: note = n is a, c, g, or t
  misc_feature 2565: note = n is a, c, g, or t
  misc_feature 2571: note = n is a, c, g, or t
  misc_feature 2574: note = n is a, c, g, or t
  misc_feature 2577: note = n is a, c, g, or t
  misc_feature 2580: note = n is a, c, g, or t
  misc_feature 2607: note = n is a, c, g, or t
  misc_feature 2625: note = n is a, c, g, or t
  misc_feature 2628: note = n is a, c, g, or t
  misc_feature 2631: note = n is a, c, g, or t
  misc_feature 2634: note = n is a, c, g, or t
  misc_feature 2637: note = n is a, c, g, or t
  misc_feature 2643: note = n is a, c, g, or t
  misc_feature 2655: note = n is a, c, g, or t
  misc_feature 2658: note = n is a, c, g, or t
  misc_feature 2661: note = n is a, c, g, or t
  misc_feature 2664: note = n is a, c, g, or t
  misc_feature 2673: note = n is a, c, g, or t
  misc_feature 2676: note = n is a, c, g, or t
  misc_feature 2697: note = n is a, c, g, or t
  misc_feature 2700: note = n is a, c, g, or t
  misc_feature 2706: note = n is a, c, g, or t
  misc_feature 2709: note = n is a, c, g, or t
  misc_feature 2712: note = n is a, c, g, or t
  misc_feature 2718: note = n is a, c, g, or t
  misc_feature 2730: note = n is a, c, g, or t
  misc_feature 2739: note = n is a, c, g, or t
  misc_feature 2742: note = n is a, c, g, or t
  misc_feature 2748: note = n is a, c, g, or t
  misc_feature 2757: note = n is a, c, g, or t
  misc_feature 2760: note = n is a, c, g, or t
  misc_feature 2763: note = n is a, c, g, or t
  misc_feature 2766: note = n is a, c, g, or t
  misc_feature 2769: note = n is a, c, g, or t
  misc_feature 2778: note = n is a, c, g, or t
  misc_feature 2781: note = n is a, c, g, or t
  misc_feature 2787: note = n is a, c, g, or t
  misc_feature 2790: note = n is a, c, g, or t
  misc_feature 2799: note = n is a, c, g, or t
  misc_feature 2802: note = n is a, c, g, or t
  misc_feature 2808: note = n is a, c, g, or t
  misc_feature 2811: note = n is a, c, g, or t
  misc_feature 2814: note = n is a, c, g, or t
  misc_feature 2820: note = n is a, c, g, or t
  misc_feature 2823: note = n is a, c, g, or t
  misc_feature 2826: note = n is a, c, g, or t
  misc_feature 2841: note = n is a, c, g, or t
  misc_feature 2847: note = n is a, c, g, or t
  misc_feature 2859: note = n is a, c, g, or t
  misc_feature 2862: note = n is a, c, g, or t
  misc_feature 2865: note = n is a, c, g, or t
  misc_feature 2868: note = n is a, c, g, or t
  misc_feature 2871: note = n is a, c, g, or t
  misc_feature 2889: note = n is a, c, g, or t
  misc_feature 2895: note = n is a, c, g, or t
  misc_feature 2898: note = n is a, c, g, or t
  misc_feature 2901: note = n is a, c, g, or t
  misc_feature 2904: note = n is a, c, g, or t
  misc_feature 2907: note = n is a, c, g, or t
  misc_feature 2916: note = n is a, c, g, or t
  misc_feature 2928: note = n is a, c, g, or t
  misc_feature 2931: note = n is a, c, g, or t
  misc_feature 2937: note = n is a, c, g, or t
  misc_feature 2940: note = n is a, c, g, or t
  misc_feature 2946: note = n is a, c, g, or t
  misc_feature 2949: note = n is a, c, g, or t
  misc_feature 2952: note = n is a, c, g, or t
  misc_feature 2958: note = n is a, c, g, or t
  misc_feature 2961: note = n is a, c, g, or t
  misc_feature 2964: note = n is a, c, g, or t
  misc_feature 2976: note = n is a, c, g, or t
  misc_feature 2979: note = n is a, c, g, or t
  misc_feature 2982: note = n is a, c, g, or t
  misc_feature 2985: note = n is a, c, g, or t
  misc_feature 2988: note = n is a, c, g, or t
  misc_feature 3015: note = n is a, c, g, or t
  misc_feature 3024: note = n is a, c, g, or t
  misc_feature 3027: note = n is a, c, g, or t
  misc_feature 3054: note = n is a, c, g, or t
  misc_feature 3060: note = n is a, c, g, or t
  misc_feature 3066: note = n is a, c, g, or t
  misc_feature 3072: note = n is a, c, g, or t
  misc_feature 3078: note = n is a, c, g, or t
  misc_feature 3081: note = n is a, c, g, or t
  misc_feature 3084: note = n is a, c, g, or t
  misc_feature 3087: note = n is a, c, g, or t
  misc_feature 3093: note = n is a, c, g, or t
  misc_feature 3096: note = n is a, c, g, or t
  misc_feature 3099: note = n is a, c, g, or t
  misc_feature 3102: note = n is a, c, g, or t
  misc_feature 3105: note = n is a, c, g, or t
  misc_feature 3111: note = n is a, c, g, or t
  misc_feature 3114: note = n is a, c, g, or t
  misc_feature 3126: note = n is a, c, g, or t
  misc_feature 3153: note = n is a, c, g, or t
  misc_feature 3159: note = n is a, c, g, or t
  misc_feature 3162: note = n is a, c, g, or t
  misc_feature 3168: note = n is a, c, g, or t
  misc_feature 3174: note = n is a, c, g, or t
  misc_feature 3177: note = n is a, c, g, or t
  misc_feature 3180: note = n is a, c, g, or t
  misc_feature 3201: note = n is a, c, g, or t
  misc_feature 3210: note = n is a, c, g, or t
  misc_feature 3222: note = n is a, c, g, or t
  misc_feature 3225: note = n is a, c, g, or t
  misc_feature 3228: note = n is a, c, g, or t
  misc_feature 3231: note = n is a, c, g, or t
  misc_feature 3234: note = n is a, c, g, or t
  misc_feature 3240: note = n is a, c, g, or t
  misc_feature 3249: note = n is a, c, g, or t
  misc_feature 3258: note = n is a, c, g, or t
  misc_feature 3264: note = n is a, c, g, or t
  misc_feature 3279: note = n is a, c, g, or t
  misc_feature 3282: note = n is a, c, g, or t
  misc_feature 3285: note = n is a, c, g, or t
  misc_feature 3288: note = n is a, c, g, or t
  misc_feature 3291: note = n is a, c, g, or t
  source 1..3294: mol_type = other DNA
atggcnaartayggngarcaygargcnwsnccngayaayggncaraaygarttywsngayathathaarwsnmgnwsngaygarcayaaygaygtncaraaraaracnttyacnaartggathaaygcnmgnttywsnaarwsnggnaarccnccnathaaygayatgttyacngayytnaargayggnmgnaarytnytngayytnytngarggnytnacnggnacnwsnytnccnaargarmgnggnwsnacnmgngtncaygcnytnaayaaygtnaaymgngtnytncargtnytncaycaraayaaygtngarytngtnaayathggnggnacngayathgtngayggnaaycayaarytnacnytnggnytnytntggwsnathathytncaytggcargtnaargaygtnatgaargaygtnatgwsngayytncarcaracnaaywsngaraarathytnytnwsntgggtnmgncaracnacnmgnccntaywsncargtnaaygtnytnaayttyacnacnwsntggacngayggnytngcnttyaaygcngtnytncaymgncayaarccngayytnttywsntgggayaargtngtnaaratgwsnccnathgarmgnytngarcaygcnttywsnaargcncaracntayytnggnathgaraarytnytngayccngargaygtngcngtncarytnccngayaaraarwsnathathatgtayytnacnwsnytnttygargtnytnccncarcargtnacnathgaygcnathmgngargtngaracnytnccnmgnaartayaaraargartgygargargargcnathaayathcarwsnacngcnccngargargarcaygarwsnccnmgngcngaracnccnwsnacngtnacngargtngayatggayytngaywsntaycarathgcnytngargargtnytnacntggytnytnwsngcngargayacnttycargarcargaygayathwsngaygaygtngargargtnaargaycarttygcnacncaygargcnttyatgatggarytnacngcncaycarwsnwsngtnggnwsngtnytncargcnggnaaycarytnathacncarggnacnytnwsngaygargargarttygarathcargarcaratgacnytnytnaaygcnmgntgggargcnytnmgngtngarwsnatggaymgncarwsnmgnytncaygaygtnytnatggarytncaraaraarcarytncarcarytnwsngcntggathcargargcngaracnacngtnaaygtnytngtngaygcnwsncaymgngaraaygcnytncargaywsnathytngcnmgngarytnaarcarcaratgcargayathcargcngarathgaygcncaycargtnaargtnaaywsnytnacncayatggtngtnathgtngaygaraaywsnggngarwsngcnacngcnathytngargaycarytncaraarytnggngarmgntggaaygayytnaargcnaarwsngcnwsnathmgngcncayytngargcnwsngcngaraartggaaymgnytnytnatgwsnytngargarytnathaartggytnaayatgaargaygargarytnaaraarcaratgccnathggnggngaygtnccngcnytncarytncartaygaycaytgyaargcnytnmgnmgngarytnaargaraargartaywsngtnytnaaygcngtngaycargcnmgngtnttyytngcngaycarccnathgargcnccngargarccnmgnmgnaayytncarwsnaaracngarytnacnccngargarmgngcncaraarathgcnaargcnatgmgnaarcarwsnwsngargtnaargaraartgggarwsnytnaaygcngtnacnwsnaaytggcaraarcargtngayaargcnytngaraarytnmgngayytncarggngcnatggaygayytngaygcngayatgaargargcngarwsngtnmgnaayggntggaarccngtnggngayytnytnathgaywsnytncargaycayathgaraarathatggcnttymgngargarathgcnccnathaayttyaargtnaaracngtnaaygayytnwsnwsncarytnwsnccnytngayytncayccnwsnytnaaratgwsnmgncarytngaygayytnaayatgmgntggaarytnytncargtnwsngtngaygaymgnytnaarcarytncargargcncaymgngayttyggnccnwsnwsncarcayttyytnwsnacnwsngtncarytnccntggcarmgnwsnathwsncayaayaargtnccntaytayathaaycaycaracncaracnacntgytgggaycayccnaaratgacngarytnttycarwsnytngcngayytnaayaaygtnmgnttywsngcntaymgnacngcnathaarathmgnmgnytncaraargcnytntgyytngayytnytngarytnwsnacnacnaaygarathttyaarcarcayaarytnaaycaraaygaycarytnytnwsngtnccngaygtnathaaytgyytnacnacnacntaygayggnytngarcaratgcayaargayytngtnaaygtnccnytntgygtngayatgtgyytnaaytggytnytnaaygtntaygayacnggnmgnacnggnaarathmgngtncarwsnytnaarathggnytnatgwsnytnwsnaarggnytnytngargaraartaymgntayytnttyaargargtngcnggnccnacngaratgtgygaycarmgncarytnggnytnytnytncaygaygcnathcarathccnmgncarytnggngargtngcngcnttyggnggnwsnaayathgarccnwsngtnmgnwsntgyttycarcaraayaayaayaarccngarathwsngtnaargarttyathgaytggatgcayytngarccncarwsnatggtntggytnccngtnytncaymgngtngcngcngcngaracngcnaarcaycargcnaartgyaayathtgyaargartgyccnathgtnggnttymgntaymgnwsnytnaarcayttyaaytaygaygtntgycarwsntgyttyttywsnggnmgnacngcnaarggncayaarytncaytayccnatggtngartaytgyathccnacnacnwsnggngar

SEQ ID NO: 5 - protein, 1108 aa, synthetic construct
  REGION 1..1108: note = amino acid sequence of nano-utrophin-2
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWIQEAETTVNVLVDASHRENALQDSILARELKQQMQDIQAEIDAHQVKVNSLTHMVVIVDENSGESATAILEDQLQKLGERWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLK

SEQ ID NO: 6 - DNA, 3327 bp, synthetic construct
  misc_feature 1..3327: note = nucleic acid sequence encoding nano-utrophin-2 protein
  source 1..3327: mol_type = other DNA
atggctaagtatggcgagcatgaggcttctcccgacaacggccagaacgagttctccgacatcatcaagtcccgctccgacgagcacaacgacgtgcagaagaaaaccttcaccaagtggatcaacgccaggttctccaagtccggcaagcctcctatcaacgacatgttcaccgacctgaaggacggccgcaaactgctggatctgctggaaggactgaccggcacctctctgcctaaagaaaggggctctactagggtgcacgccctgaacaatgtgaacagggtgctccaggtgctgcaccagaacaacgtcgagctggtcaatatcggcggcaccgacatcgtggatggcaaccataagctgaccctgggcctgctgtggtccatcattctgcactggcaggtcaaagacgtgatgaaggacgtgatgtccgacctccagcagaccaactccgagaagatcctgctgtcttgggtccgccagaccactaggccctattctcaggtcaacgtgctgaacttcaccacctcttggaccgacggactggccttcaatgctgtgctgcacaggcacaagcccgacctgttctcttgggacaaagtggtcaagatgagccccatcgagaggctggaacacgccttctctaaggcccagacctacctgggaatcgagaagctgctggaccctgaggatgtggcagtgcagctgcccgacaagaaatccatcatcatgtacctgaccagcctgttcgaggtgctgcctcagcaggtcacaattgatgccatccgcgaggtggaaaccctgcctcgcaagtacaagaaagagtgcgaggaagaggccatcaacatccagtccactgctcccgaggaagaacacgagtctccaagggccgagactccttccaccgtgactgaagtggacatggacctggactcctaccagatcgctctggaagaggtgctgacttggctgctgtccgccgaggatactttccaggaacaggacgacatctccgacgacgtcgaggaagtgaaggaccagttcgccactcacgaggctttcatgatggaactgactgcccaccagtcctccgtgggaagtgtcctccaggctggaaaccagctgatcacccagggaaccctgtccgatgaggaagagttcgagattcaggaacagatgaccctgctgaacgccagatgggaagctctgagggtcgagtccatggataggcagtctaggctgcacgacgtgctcatggaactccagaagaagcagctccagcagctgtccgcttggatccaggaagccgagactaccgtcaatgtgctggtggacgcctctcacagagagaacgccctccaggattccatcctggctagggaactgaagcagcagatgcaggacatccaggccgagatcgacgctcaccaggtcaaagtgaactccctgactcacatggtggtcatcgtggacgagaattccggcgagtctgccaccgctatcctggaagatcagctccagaaactgggcgagcgctggaacgatctgaaggctaagtccgcctctatcagggcccatctggaagcctctgccgagaagtggaataggctgctgatgtccctggaagaactgatcaagtggctgaacatgaaggatgaggaactcaagaaacagatgcccatcggaggcgacgtgccagctctccagctccagtatgatcattgcaaggctctgaggcgcgagctgaaagagaaagagtactccgtcctgaacgccgtggatcaggctagggtgttcctggccgatcagcctattgaggctcctgaggaacctaggcggaacctccagtccaagaccgagctgactcctgaagagagggcccagaaaatcgccaaggctatgcgcaagcagtccagcgaagtcaaagagaagtgggagtccctgaatgccgtgacctccaactggcagaaacaggtggacaaggccctggaaaagctgagggatctccagggcgctatggacgacctggacgccgatatgaaggaagctgagtccgtcagaaacggctggaagccagtgggagatctgctgattgatagcctccaggaccacattgagaagatcatggccttcagggaagagatcgcccctatcaacttcaaagtcaagaccgtgaacgacctgtccagccagctgagccctctggatctccatccaagcctgaagatgagcaggcagctggatgacctgaacatgcgctggaaactcctccaggtgtccgtggacgataggctgaaacagctccaggaagctcacagagacttcggccctagctctcagcacttcctgtctacctctgtgcagctcccttggcagcgctccatctctcacaacaaggtgccctactacatcaaccaccagactcagaccacctgttgggatcaccccaagatgactgagctgttccagagcctggccgacctcaacaatgtgcgcttctccgcctataggaccgccatcaaaatcaggcgcctccagaaagccctgtgcctggacctgctcgaactgtccaccaccaacgagatcttcaagcagcacaagctgaaccagaacgaccagctgctgtctgtgcccgacgtgatcaattgcctgaccaccacctacgacgggctcgaacagatgcacaaggatctcgtgaatgtgcctctgtgcgtggacatgtgcctgaactggctgctgaatgtgtacgataccggcaggaccggcaagatcagggtgcagtctctgaagatcggcctgatgagcctgagcaaggggctgctcgaagagaagtaccgctacctgttcaaagaggtggcaggccctaccgagatgtgcgatcagagacagctgggactgctgctgcacgatgccattcagattcctcgccagctgggagaagtggccgccttcggaggatctaatatcgagccttccgtgcgctcctgctttcagcagaacaacaacaagcctgagatctccgtgaaagaattcatcgactggatgcacctggaacctcagagcatggtctggctgccagtcctgcatagagtggctgctgctgagactgccaagcaccaggccaagtgcaacatctgcaaagagtgccctatcgtcggcttccgctatcgctccctgaagcacttcaactacgacgtgtgccagagttgcttcttttccggacgcaccgccaagggccacaagctgcattaccctatggtcgagtactgcatccctaccacatccggcgaggatgtgcgcgatttcaccaaggtgctgaagtga

SEQ ID NO: 7 - protein, 1142 aa, synthetic construct
  REGION 1..1142: note = amino acid sequence of nano-utrophin-3
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWIQEAETTVNVLVDASHRENALQDSILARELKQQMQDIQAEIDAHQVKVNSLTHMVVIVDENSGESATAILEDQLQKLGERWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETPIT

SEQ ID NO: 8 - DNA, 3429 bp, synthetic construct
  misc_feature 1..3429: note = nucleic acid sequence encoding nano-utrophin-3 protein
  source 1..3429: mol_type = other DNA
atggctaagtatggcgagcatgaggcttctcccgacaacggccagaacgagttctccgacatcatcaagtcccgctccgacgagcacaacgacgtgcagaagaaaaccttcaccaagtggatcaacgccaggttctccaagtccggcaagcctcctatcaacgacatgttcaccgacctgaaggacggccgcaaactgctggatctgctggaaggactgaccggcacctctctgcctaaagaaaggggctctactagggtgcacgccctgaacaatgtgaacagggtgctccaggtgctgcaccagaacaacgtcgagctggtcaatatcggcggcaccgacatcgtggatggcaaccataagctgaccctgggcctgctgtggtccatcattctgcactggcaggtcaaagacgtgatgaaggacgtgatgtccgacctccagcagaccaactccgagaagatcctgctgtcttgggtccgccagaccactaggccctattctcaggtcaacgtgctgaacttcaccacctcttggaccgacggactggccttcaatgctgtgctgcacaggcacaagcccgacctgttctcttgggacaaagtggtcaagatgagccccatcgagaggctggaacacgccttctctaaggcccagacctacctgggaatcgagaagctgctggaccctgaggatgtggcagtgcagctgcccgacaagaaatccatcatcatgtacctgaccagcctgttcgaggtgctgcctcagcaggtcacaattgatgccatccgcgaggtggaaaccctgcctcgcaagtacaagaaagagtgcgaggaagaggccatcaacatccagtccactgctcccgaggaagaacacgagtctccaagggccgagactccttccaccgtgactgaagtggacatggacctggactcctaccagatcgctctggaagaggtgctgacttggctgctgtccgccgaggatactttccaggaacaggacgacatctccgacgacgtcgaggaagtgaaggaccagttcgccactcacgaggctttcatgatggaactgactgcccaccagtcctccgtgggaagtgtcctccaggctggaaaccagctgatcacccagggaaccctgtccgatgaggaagagttcgagattcaggaacagatgaccctgctgaacgccagatgggaagctctgagggtcgagtccatggataggcagtctaggctgcacgacgtgctcatggaactccagaagaagcagctccagcagctgtccgcttggatccaggaagccgagactaccgtcaatgtgctggtggacgcctctcacagagagaacgccctccaggattccatcctggctagggaactgaagcagcagatgcaggacatccaggccgagatcgacgctcaccaggtcaaagtgaactccctgactcacatggtggtcatcgtggacgagaattccggcgagtctgccaccgctatcctggaagatcagctccagaaactgggcgagcgctggaacgatctgaaggctaagtccgcctctatcagggcccatctggaagcctctgccgagaagtggaataggctgctgatgtccctggaagaactgatcaagtggctgaacatgaaggatgaggaactcaagaaacagatgcccatcggaggcgacgtgccagctctccagctccagtatgatcattgcaaggctctgaggcgcgagctgaaagagaaagagtactccgtcctgaacgccgtggatcaggctagggtgttcctggccgatcagcctattgaggctcctgaggaacctaggcggaacctccagtccaagaccgagctgactcctgaagagagggcccagaaaatcgccaaggctatgcgcaagcagtccagcgaagtcaaagagaagtgggagtccctgaatgccgtgacctccaactggcagaaacaggtggacaaggccctggaaaagctgagggatctccagggcgctatggacgacctggacgccgatatgaaggaagctgagtccgtcagaaacggctggaagccagtgggagatctgctgattgatagcctccaggaccacattgagaagatcatggccttcagggaagagatcgcccctatcaacttcaaagtcaagaccgtgaacgacctgtccagccagctgagccctctggatctccatccaagcctgaagatgagcaggcagctggatgacctgaacatgcgctggaaactcctccaggtgtccgtggacgataggctgaaacagctccaggaagctcacagagacttcggccctagctctcagcacttcctgtctacctctgtgcagctcccttggcagcgctccatctctcacaacaaggtgccctactacatcaaccaccagactcagaccacctgttgggatcaccccaagatgactgagctgttccagagcctggccgacctcaacaatgtgcgcttctccgcctataggaccgccatcaaaatcaggcgcctccagaaagccctgtgcctggacctgctcgaactgtccaccaccaacgagatcttcaagcagcacaagctgaaccagaacgaccagctgctgtctgtgcccgacgtgatcaattgcctgaccaccacctacgacgggctcgaacagatgcacaaggatctcgtgaatgtgcctctgtgcgtggacatgtgcctgaactggctgctgaatgtgtacgataccggcaggaccggcaagatcagggtgcagtctctgaagatcggcctgatgagcctgagcaaggggctgctcgaagagaagtaccgctacctgttcaaagaggtggcaggccctaccgagatgtgcgatcagagacagctgggactgctgctgcacgatgccattcagattcctcgccagctgggagaagtggccgccttcggaggatctaatatcgagccttccgtgcgctcctgctttcagcagaacaacaacaagcctgagatctccgtgaaagaattcatcgactggatgcacctggaacctcagagcatggtctggctgccagtcctgcatagagtggctgctgctgagactgccaagcaccaggccaagtgcaacatctgcaaagagtgccctatcgtcggcttccgctatcgctccctgaagcacttcaactacgacgtgtgccagagttgcttcttttccggacgcaccgccaagggccacaagctgcattaccctatggtcgagtactgcatccctaccacatccggcgaggatgtgcgcgatttcaccaaggtgctgaagaacaagttccgctccaagaagtatttcgctaagcaccccaggctgggctatctgcctgtccagactgtcctggaaggcgacaacctggaaacccctatcacctga

SEQ ID NO: 9 - protein, 736 aa, synthetic construct
  REGION 1..736: note = amino acid sequence with GenBank accession: AAS99264
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 2211 bp, synthetic construct
  misc_feature 1..2211: note = coding sequence for AAV9 capsid vp1
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 11 - DNA, 2211 bp, synthetic construct; identical to SEQ ID NO: 10
  misc_feature 1..2211: note = coding sequence for hu31 vp1
  source 1..2211: mol_type = other DNA

SEQ ID NO: 12 - DNA, 2211 bp, synthetic construct
  misc_feature 1..2211: note = coding sequence for hu32 vp1
  source 1..2211: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 13 - protein, 1131 aa, synthetic construct
  REGION 1..1131: note = amino acid sequence for nano-dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGDLEQEQVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 14 - protein, 1131 aa, synthetic construct
  REGION 1..1131: note = amino acid sequence for nano-dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGELEQEQVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 15 - protein, 1131 aa, synthetic construct; identical to SEQ ID NO: 1
  REGION 1..1131: note = amino acid sequence for nano-dystrophin

SEQ ID NO: 16 - protein, 1131 aa, synthetic construct
  REGION 1..1131: note = amino acid sequence for nano-dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGEIEAEQVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 17 - protein, 1131 aa, synthetic construct
  REGION 1..1131: note = amino acid sequence for nano-dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGEIEAHQVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 18 - protein, 1131 aa, synthetic construct
  REGION 1..1131: note = amino acid sequence for nano-dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTEAETTANVLQDATRKERLLEDSKGVKELMKQWQDLQGEIEAHTVRVNSLTHMVVVVDESSGDHATAALEEQLKVLGDRWANICRWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 19 - DNA, 3420 bp, synthetic construct
  misc_feature 1..3420: note = engineered suquence encoding nano-utrophin
  source 1..3420: mol_type = other DNA
  CDS 1..3420
atggctaagtatggcgagcatgaggcttctcccgacaacggccagaacgagttctccgacatcatcaagtcccgctccgacgagcacaacgacgtgcagaagaaaaccttcaccaagtggatcaacgccaggttctccaagtccggcaagcctcctatcaacgacatgttcaccgacctgaaggacggccgcaaactgctggatctgctggaaggactgaccggcacctctctgcctaaagaaaggggctctactagggtgcacgccctgaacaatgtgaacagggtgctccaggtgctgcaccagaacaacgtcgagctggtcaatatcggcggcaccgacatcgtggatggcaaccataagctgaccctgggcctgctgtggtccatcattctgcactggcaggtcaaagacgtgatgaaggacgtgatgtccgacctccagcagaccaactccgagaagatcctgctgtcttgggtccgccagaccactaggccctattctcaggtcaacgtgctgaacttcaccacctcttggaccgacggactggccttcaatgctgtgctgcacaggcacaagcccgacctgttctcttgggacaaagtggtcaagatgagccccatcgagaggctggaacacgccttctctaaggcccagacctacctgggaatcgagaagctgctggaccctgaggatgtggcagtgcagctgcccgacaagaaatccatcatcatgtacctgaccagcctgttcgaggtgctgcctcagcaggtcacaattgatgccatccgcgaggtggaaaccctgcctcgcaagtacaagaaagagtgcgaggaagaggccatcaacatccagtccactgctcccgaggaagaacacgagtctccaagggccgagactccttccaccgtgactgaagtggacatggacctggactcctaccagatcgctctggaagaggtgctgacttggctgctgtccgccgaggatactttccaggaacaggacgacatctccgacgacgtcgaggaagtgaaggaccagttcgccactcacgaggctttcatgatggaactgactgcccaccagtcctccgtgggaagtgtcctccaggctggaaaccagctgatcacccagggaaccctgtccgatgaggaagagttcgagattcaggaacagatgaccctgctgaacgccagatgggaagctctgagggtcgagtccatggataggcagtctaggctgcacgacgtgctcatggaactccagaagaagcagctccagcagctgtccgcttggatccaggaagccgagactaccgtcaatgtgctggtggacgcctctcacagagagaacgccctccaggattccatcctggctagggaactgaagcagcagatgcaggacatccaggccgagatcgacgctcaccaggtcaaagtgaactccctgactcacatggtggtcatcgtggacgagaattccggcgagtctgccaccgctatcctggaagatcagctccagaaactgggcgagcgctggaacgatctgaaggctaagtccgcctctatcagggcccatctggaagcctctgccgagaagtggaataggctgctgatgtccctggaagaactgatcaagtggctgaacatgaaggatgaggaactcaagaaacagatgcccatcggaggcgacgtgccagctctccagctccagtatgatcattgcaaggctctgaggcgcgagctgaaagagaaagagtactccgtcctgaacgccgtggatcaggctagggtgttcctggccgatcagcctattgaggctcctgaggaacctaggcggaacctccagtccaagaccgagctgactcctgaagagagggcccagaaaatcgccaaggctatgcgcaagcagtccagcgaagtcaaagagaagtgggagtccctgaatgccgtgacctccaactggcagaaacaggtggacaaggccctggaaaagctgagggatctccagggcgctatggacgacctggacgccgatatgaaggaagctgagtccgtcagaaacggctggaagccagtgggagatctgctgattgatagcctccaggaccacattgagaagatcatggccttcagggaagagatcgcccctatcaacttcaaagtcaagaccgtgaacgacctgtccagccagctgagccctctggatctccatccaagcctgaagatgagcaggcagctggatgacctgaacatgcgctggaaactcctccaggtgtccgtggacgataggctgaaacagctccaggaagctcacagagacttcggccctagctctcagcacttcctgtctacctctgtgcagctcccttggcagcgctccatctctcacaacaaggtgccctactacatcaaccaccagactcagaccacctgttgggatcaccccaagatgactgagctgttccagagcctggccgacctcaacaatgtgcgcttctccgcctataggaccgccatcaaaatcaggcgcctccagaaagccctgtgcctggacctgctcgaactgtccaccaccaacgagatcttcaagcagcacaagctgaaccagaacgaccagctgctgtctgtgcccgacgtgatcaattgcctgaccaccacctacgacgggctcgaacagatgcacaaggatctcgtgaatgtgcctctgtgcgtggacatgtgcctgaactggctgctgaatgtgtacgataccggcaggaccggcaagatcagggtgcagtctctgaagatcggcctgatgagcctgagcaaggggctgctcgaagagaagtaccgctacctgttcaaagaggtggcaggccctaccgagatgtgcgatcagagacagctgggactgctgctgcacgatgccattcagattcctcgccagctgggagaagtggccgccttcggaggatctaatatcgagccttccgtgcgctcctgctttcagcagaacaacaacaagcctgagatctccgtgaaagaattcatcgactggatgcacctggaacctcagagcatggtctggctgccagtcctgcatagagtggctgctgctgagactgccaagcaccaggccaagtgcaacatctgcaaagagtgccctatcgtcggcttccgctatcgctccctgaagcacttcaactacgacgtgtgccagagttgcttcttttccggacgcaccgccaagggccacaagctgcattaccctatggtcgagtactgcatccctaccacatccggcgaggatgtgcgcgatttcaccaaggtgctgaagaacaagttccgctccaagaagtatttcgctaagcaccccaggctgggctatctgcctgtccagactgtcctggaaggcgacaacctggaaacctga

SEQ ID NO: 20 - protein, 1139 aa, synthetic construct
  REGION 1..1139: note = Synthetic Construct
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLLSAEDTFQEQDDISDDVEEVKDQFATHEAFMMELTAHQSSVGSVLQAGNQLITQGTLSDEEEFEIQEQMTLLNARWEALRVESMDRQSRLHDVLMELQKKQLQQLSAWIQEAETTVNVLVDASHRENALQDSILARELKQQMQDIQAEIDAHQVKVNSLTHMVVIVDENSGESATAILEDQLQKLGERWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLET

SEQ ID NO: 21 - protein, 1208 aa, synthetic construct
  REGION 1..1208: note = human utrophin mutant
  REGION 1..324: note = MISC_FEATURE - amino acid sequence identical to N-terminal region of full length human utrophin
  REGION 311..417: note = MISC_FEATURE - hybrid triple helix
  REGION 325..362: note = MISC_FEATURE - amino acid sequence identical to C-terminal region of full length human utrophin
  REGION 363..401: note = MISC_FEATURE - amino acid sequence identical to N-terminal region of full length human utrophin
  REGION 402..1208: note = MISC_FEATURE - amino acid sequence identical to C-terminal region of full length human utrophin
MAKYGEHEASPDNGQNEFSDIIKSRSDEHNDVQKKTFTKWINARFSKSGKPPINDMFTDLKDGRKLLDLLEGLTGTSLPKERGSTRVHALNNVNRVLQVLHQNNVELVNIGGTDIVDGNHKLTLGLLWSIILHWQVKDVMKDVMSDLQQTNSEKILLSWVRQTTRPYSQVNVLNFTTSWTDGLAFNAVLHRHKPDLFSWDKVVKMSPIERLEHAFSKAQTYLGIEKLLDPEDVAVQLPDKKSIIMYLTSLFEVLPQQVTIDAIREVETLPRKYKKECEEEAINIQSTAPEEEHESPRAETPSTVTEVDMDLDSYQIALEEVLTWLVLIDQMLKSNIVTVGDVEEINKTVSRMKITKADLEQRQSSVGSVLQAGNQLITQGTLSDEEEFEIQEQMTLLNARWDGTQHGVELRQQQLEDMIIDSLQWDDHREETEELMRKYEARLYILQQARRDPLTKQISDNQILLQELGPGDGIVMAFDNVLQKLLEEYGSDDTRNVKETTEYLKTSWINLKQSIADRQNALEAEWRTVQASRRDLENFLKWIQEAETTVNVLVDASHRENALQDSILARELKQQMQDIQAEIDAHNDIFKSIDGNRQKMVKALGNSEEATMLQHRLDDMNQRWNDLKAKSASIRAHLEASAEKWNRLLMSLEELIKWLNMKDEELKKQMPIGGDVPALQLQYDHCKALRRELKEKEYSVLNAVDQARVFLADQPIEAPEEPRRNLQSKTELTPEERAQKIAKAMRKQSSEVKEKWESLNAVTSNWQKQVDKALEKLRDLQGAMDDLDADMKEAESVRNGWKPVGDLLIDSLQDHIEKIMAFREEIAPINFKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELSTTNEIFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKEFIDWMHLEPQSMVWLPVLHRVAAAETAKHQAKCNICKECPIVGFRYRSLKHFNYDVCQSCFFSGRTAKGHKLHYPMVEYCIPTTSGE

SEQ ID NO: 22 - protein, 1237 aa, synthetic construct
  REGION 1..1237: note = mutant human dystrophin protein
  REGION 1..354: note = MISC_FEATURE - amino acid sequence identical to N-terminal region of full length human dystrophin
  REGION 340..447: note = MISC_FEATURE - hybrid triple helix
  REGION 355..392: note = MISC_FEATURE - amino acid sequence identical to C-terminal region of full length human dystrophin
  REGION 393..431: note = MISC_FEATURE - amino acid sequence identical to N-terminal region of full length human dystrophin
  REGION 432..1237: note = MISC_FEATURE - amino acid sequence identical to C-terminal region of full length human dystrophin
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLSLLDQVIKSQRVMVGDLEDINEMIIKQKATMQDLEQRQGRVGNILQLGSKLIGTGKLSEDEETEVQEQMNLLNSRWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGE

SEQ ID NO: 23 - protein, 12 aa, synthetic construct
  REGION 1..12: note = synthetic construct
LQGEIEAHTDVY

SEQ ID NO: 24 - protein, 9 aa, synthetic construct
  REGION 1..9: note = synthetic construct
QEDLEQEQV

SEQ ID NO: 25 - protein, 1164 aa, synthetic construct
  REGION 1..1164: note = synthetic construct
MAKYGEHEARPDDGQNEFSDIIKSRSDEHNDVQKKTFTKWINARFSKSGKPPINDMFTDLKDGRKLLDLLEGLTGTSLPKERGSTRVHALNNVNRVLQVLHQNNVDLVNIGGTDIVDGNHKLTLGLLWSIILHWQVKDVMKDVMSDLQQTNSEKILLSWVRQSTRPYSQVNVLNFTTSWTDGLAFNAVLHRHKPDLFSWDRVVKMSPIERLEHAFSKADTYLGIEKLLDPEDVAVQLPDKKSIIMYLTSLFEVLPQQVTLDAIREVETLPRKYKKECEEGEISIQSSAPEEEHECPGAETPSTVTEVDTDLDSYQIALEEVLTWLLSAEDTFQEQDDISDDVEEVKEQFTTHEAFMMELTAHQSSVGSVLQAGNQLITQGTLSDEEEFEIQEQMTLLNARWEALRNDSMNRQSRLKDVLMELQKKQLQQLSAWLTLTEERIQKMETCPLDDDLKSLQKLLEDHKRLQNDLEAEQVKVNSLTHMVVIVDENSGESATAVLEDQLQKLGERWTAVCRWTEERWSRLQEINILWQELLEEQCLLKAWLTEKEEALNKVQTSNFKDQKELSVSIRRLAILKEDMEMKRQALDQLSELGQDVGQLVDNPKASKKINSDSEELTQRWDSLVQRLEDSSNQVTQAVAKLGMSQIPQKDLLETVRIREQVTTKRSKQELPPPPPPKKRQIPVDLEKLRDLQGAMDDLDVDMKEAEAVRNGWKPVGDLLIDSLQDHIEKTMAFREEIAPINLKVKTVNDLSSQLSPLDLHPSLKMSRQLDDLNMRWKLLQVSVDDRLKQLQEAHRDFGPSSQHFLSTSVQLPWQRSISHNKVPYYINHQTQTTCWDHPKMTELFQSLADLNNVRFSAYRTAIKIRRLQKALCLDLLELNTTNEVFKQHKLNQNDQLLSVPDVINCLTTTYDGLEQMHKDLVNVPLCVDMCLNWLLNVYDTGRTGKIRVQSLKIGLMSLSKGLLEEKYRYLFKEVAGPTEMCDQRQLGLLLHDAIQIPRQLGEVAAFGGSNIEPSVRSCFQQNNNKPEISVKDFIDWMRLEPQSMVWLPVLHRVAAAETAKHQAKCNICKECPIVGFRYRSLKHFNYDVCQSCFFSGRTAKGHKLHYPMVEYCIPTTSGEDVRDFTKVLKNKFRSKKYFAKHPRLGYLPVQTVLEGDNLETQAM